(19) United States
(12) Patent Application Publication
(10) Pub. No.: US 2024/0084272 A1
(43) Pub. Date: Mar. 14, 2024

(54) CYTOCHROME P450 MONOOXYGENASES AND USES THEREOF

(71) Applicant: THE UNIVERSITY OF BRITISH COLUMBIA, Vancouver (CA)

(72) Inventors: Thu-Thuy T. Dang, Vancouver (CA); Trinh-don Nguyen, Vancouver (CA); Tuan-ahn M. Nguyen, Vancouver (CA); Yuen Yee Leung, Vancouver (CA)

(21) Appl. No.: 18/266,787

(22) PCT Filed: Dec. 10, 2021
(86) PCT No.: PCT/CA2021/051778

§ 371 Date: Jun. 12, 2023

Related U.S. Application Data
(60) Provisional application No. 63/123,678, filed on Dec. 10, 2020.

(51) Int. Cl. C12N 9/02; A61K 31/437; A61P 35/00; C12P 17/18

(52) U.S. Cl. CPC C12N 9/0071; A61K 31/437; A61P 35/00; C12P 17/18; C12Y 114/14001

(57) ABSTRACT

The present disclosure relates to cytochrome P450 monooxygenases capable of oxidizing a monoterpenoid indole alkaloid (MIA) substrate and methods and uses thereof. The substrate may be a camptothecinoid, an evodiaminoid or an ellipticinoid. The disclosure further relates to method of producing hydroxylated monoterpenoid indole alkaloids, as well as derivatives and analogues of the produced hydroxylated monoterpenoid indole alkaloids. Pharmaceutical compositions comprising the hydroxylated monoterpenoid indole alkaloid derivatives are also provided.

### FIELD OF INVENTION

The present disclosure relates to cytochrome P450 monooxygenases capable of oxidizing monoterpenoid indole alkaloid (MIA) substrates. Method of use and novel compounds produced by the method are also provided.

### BACKGROUND

Quinoline alkaloid camptothecin (CPT, (1)), first extracted from the stems of Camptotheca acuminata (also known in traditional Chinese medicine as happy tree, “xi shù”/“ [symbol] ”) in 1966 (Wall et al., Journal of the American Chemical Society (1966) 88(1)), serves as a lead compound for designing many more active and clinically useful anticancer drugs, such as irinotecan (7-ethyl-10-[4-(1-piperidino)-1-piperidino]-carbonyloxycamptothecine; trade-name: Camptosar) and topotecan (9-[(dimethylamino)-methyl]-10-hydroxycamptothecin; trade-name: Hycamtin) (Dwyer et al 2006, J. Clin. Oncol. 24, 4534-8). CPT and its derivatives are potent inhibitors of DNA topoisomerase I activity and are widely used for the treatment of lung, cervix, ovarian and colon cancers (Lorence and Nessler 2004, Phytochemistry. 65, 2735-2749), and other diseases such as acquired immune deficiency syndrome (AIDS) and falciparum malaria. Traditional production strategies of CPT and derivatives based on isolation from natural sources and chemical derivatization and modification present many challenges associated with the purity, scale, and complexity of the compounds, contributing to their rising costs and inaccessibility. Topotecan (4) and irinotecan (3) are currently approved in many countries for the treatment of metastatic ovarian cancer, cervical, uterine, and brain cancers among others. Despite several reports on the total synthesis of camptothecin and its derivatives, irinotecan and topotecan are semisynthesized from plant-extracted camptothecin for commercial use (Comins and Nolan 2001, Org. Lett. 3, 4255-4257; Shweta et al 2010, Phytochemistry. 71, 117-22). Partial synthetic approaches that rely on camptothecin precursors from plants (Puri et al, WO2004087715A1) require more than four steps with low yields and harsh chemical conditions (Chavan and Sivappa 2004, Tetrahedron Lett. 45, 3113-3115). It is highlighted that the semisynthesis of topotecan from camptothecin can only be achieved via 10-hydroxycamptothecin ((2), 10HCPT). This reaction includes the two-step reduction-oxidation process involving an initial Pt-catalyzed partial hydrogenation to tetrahydroquinoline intermediate and its subsequent oxidation with extremely toxic Pb(OAc) to yield 10HCPT (Li et al. 2016, Angew. Chemie Int. Ed. 55, 14778-14783). Subsequent condensation of 10HCPT with formaldehyde and dimethylamine give topotecan (Kingsbury et al 1991, J. Med. Chem. 34, 98-107). Although this method can deliver topotecan at economically feasible yields, it involves time consuming and labour intensive processes (costing 1-3 days of work) and toxic and dangerous chemicals on industrial scale (Pb(OAc)₄, H₂O₂). The pharmaceutical industry has been relying on HCPT for the semisynthesis for many C10-modified camptothecin analogs such as topotecan (Kingsbury et al 1991), irinotecan (Hu and Ham, WO2008127606A1), and SN-38 (7-ethyl-10HCPT). C. acuminata does produce HCPT but at very low level (0.003%) and in limited locations, including in the bark, young leaves and seeds (Kacprzak 2013, Chemistry and Biology of Camptothecin and its Derivatives,. In Natural Products 643-682 (Springer Berlin Heidelberg); Salim et al. 2018 The Plant Journal 95, 112-125). The low level of HCPT in the plant and the difficult partial chemical synthesis make it challenging to access derivatives of CPT. Moreover, other CPT derivatives such as 11-hydroxycamptothecin (11HCPT, (5)), which exhibits a much greater therapeutic index than CPT, occur in even lower quantities (Wall et al 1986, Journal of Medicinal Chemistry 29, 1553-1555), limiting their clinical use. Today, about 600 kg of plant-extracted CPT are produced each year, which (i) does not meet the demand for the synthesis of CPT derivatives (currently, about 3,000 kg/year) and (ii) leads to destructive harvesting of C. acuminata and Nothapodytes foetida trees, potentially restricting future supplies of CPT-derived drugs.

The low level of 10HCPT and 11HCPT in the plant and the difficult partial chemical synthesis make it challenging to access and diversify more CPT derivatives, especially the 11HCPT analogues. There is therefore a need to find new and economically viable ways to produce camptothecin derivatives and analogues.

### SUMMARY OF THE INVENTION

The present disclosure relates to cytochrome P450 monooxygenases capable of oxidizing monoterpenoid indole alkaloid (MIA) substrates. Method of use and novel compounds produced by the method are also provided.

In one aspect it is provided cytochrome P450 monooxygenase capable of oxidizing a monoterpenoid indole alkaloid (MIA) substrate, wherein the MIA substrate comprises a quinoline moiety or an indole moiety. The MIA substrate may comprises a camptothecinoid, evodiaminoid or ellipticinoid. In one aspect the MIA substrate may be camptothecin, 7-ethylcamptothecin, 9-amino-camptothecin, 9-nitro-camptothecin, 9-hydroxycamptothecin, 10-hydroxycamptothecin, 11-hydroxycamptothecin, evodiamine or ellipticine.

The cytochrome P450 monooxygenase may be a camptothecin hydroxylase. The camptothecin hydroxylase may be CPT 9-hydroxylase (CPT9H), CPT 10-hydroxylase (CPT10H) or CPT 11-hydroxylase (CPT11H). The camptothecin hydroxylase may be derived from Camptotheca acuminata, Ophiorrhiza pumila or Nothapodytes nimmoniana or the camptothecin hydroxylase may be derived from an orthologue or homolog of the camptothecin hydroxylase from Camptotheca acuminata, Ophiorrhiza pumila or Nothapodytes nimmoniana.

The cytochrome P450 monooxygenase may comprise sequence with 80-100% identity to SEQ ID NO: 3, 4, 8, 9, 10, 14, 15, 16, 18, 20, 22, 24, 26, 28 or 30, or an active fragment or variant thereof.

Further provided is a nucleic acid encoding the cytochrome P450 monooxygenase as described above.

In another aspect it is provided a transgenic host or host cell comprising the cytochrome P450 monooxygenase as described above. The host cell may also comprise the nucleic acid encoding the cytochrome P450 monooxygenase as described above. The host or host cell may be a bacterial, fungal, yeast, algae, diatom, plant, insect, amphibian, or animal transgenic host or host cell.

In a further aspect it is provided a method (A) of producing a hydroxylated monoterpenoid indole alkaloid (MIA), wherein the MIA comprises a quinoline moiety or an indole moiety, the method comprising (a) providing a first cytochrome P450 monooxygenase, wherein the first cytochrome P450 monooxygenase comprises the cytochrome P450 monooxygenase as described above and (b) contacting a monoterpenoid indole alkaloid (MIA) substrate with the first cytochrome P450 monooxygenase under conditions suitable for oxidation or hydroxylation of the MIA substrate to produce a hydroxylated MIA. The MIA substrate in the method may be a camptothecinoid, evodiaminoid or ellipticinoid. In another aspect the MIA substrate used in the method may be camptothecine, 7-ethylcamptothecin, 9-amino-camptothecin, 10-hydroxycamptothecin, evodiamine or ellipticine. The first cytochrome P450 monooxygenase may be CPT 9-hydroxylase, CPT 10-hydroxylase or CPT 11-hydroxylase.

The method may further comprises contacting the hydoxylated MIA with a second cytochrome P450 monooxygenase, wherein the second cytochrome P450 monooxygenase as described above, under conditions suitable for oxidation or hydroxylation of the hydroxylated MIA to produce a dihydroxylated MIA. In one aspect the first cytochrome P450 monooxygenase is a CPT 10-hydroxylase and the second cytochrome P450 monooxygenase is a CPT 11-hydroxylase.

In another aspect it is provided a method (B) of producing a hydroxylated monoterpenoid indole alkaloid (MIA), the method comprising: (a) providing a transgenic host or host cell, wherein the transgenic host or host cell comprises the cytochrome P450 monooxygenase as described above and/or wherein the transgenic host or host cell comprise the nucleic acid encoding the cytochrome P450 monooxygenase as described above; (b) incubating the host or host cell under condition suitable for the expression of the cytochrome P450 monooxygenases; and (c) contacting the cytochrome P450 monooxygenases with a MIA substrate under conditions suitable for oxidation or hydroxylation of the MIA substrate to produce a hydroxylated MIA product. The contacting in step (c) may comprises an in vitro contact or the contacting in step (c) may comprises an in vivo contact within the host or host cell.

Method (A) or method (B) may further comprising the step of recovering the hydroxylated MIA.

The MIA substrate used in method (A) or method (B) may be a camptothecinoid, an evodiaminoid or an ellipticinoid. In an aspect the MIA substrate may be camptothecin, 9-amino-camptothecin, 10-hydroxycamptothecin, 7 ethyl camptothecin or 9-nitro-camptothecin.

The hydroxylated monoterpenoid indole alkaloid (MIA) produced by method (A) or method (B) may be a 9-hydroxycamptothecinoid, a 10-hydroxycamptothecinoid, a 11-hydroxycamptothecinoid, 10,11-dihydroxycamptothecinoid, a 7-ethyl-10-hydroxycamptothecinoid, a 9-amino-hydroxycamptothecinoid, a 9-nitro-hydroxycamptothecinoid or a combination thereof.

The hydroxylated MIA product produced by method (A) or method (B) may further be processed into a MIA derivative.

In one aspect the MIA derivative may be a camptothecin analogue selected from: 9-[(dimethylamino)methyl]-10-hydroxycamptothecin (topotecan); 12-[(dimethylamino)methyl]-11-hydroxycamptothecin (topotecan-11), 7-ethyl-10-[4-(1-piperidino)-1-piperidino]carbonyloxycamptothecin (irinotecan); 7-ethyl-11-[4-(1-piperidino)-1-piperidino]carbonyloxycamptothecin (irinotecan-11); 7-ethyl-10-hydroxycamptothecin; 7-ethyl-11-hydroxycamptothecin; 9-bromo-10-hydroxycamptothecin; 12-bromo-10-hydroxycamptothecin; 9-amino-10-hydroxycamptothecin or 9-amino-11-hydroxycamptothecin.

In another aspect it is provided a monoterpenoid indole alkaloid (MIA) derivative produced from the hydroxylated MIA product produced by method (A) or method (B). The MIA derivative may be 12-[(dimethylamino)methyl]-11-hydroxycamptothecin (topotecan-11), 7-ethyl-11-[4-(1-piperidino)-1-piperidino]carbonyloxycamptothecin (irinotecan-11), 10,11-dihydroxycamptothecin or 12-bromo-11-hydroxycamptothecin, 10-hydroxy-11-methoxycamptothecin or 11-hydroxy-10-methoxycamptothecin.

In yet another aspect it is provided a camptothecin derivative having the chemical structure of Formula I:

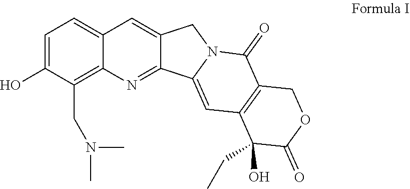 [SMILES: CC[C@@]1(O)C(=O)OCc2c1cc1n(c2=O)Cc2cc3ccc(O)c(CN(C)C)c3nc2-1]

In a further aspect it is provided a camptothecin derivative having the chemical structure of Formula II:

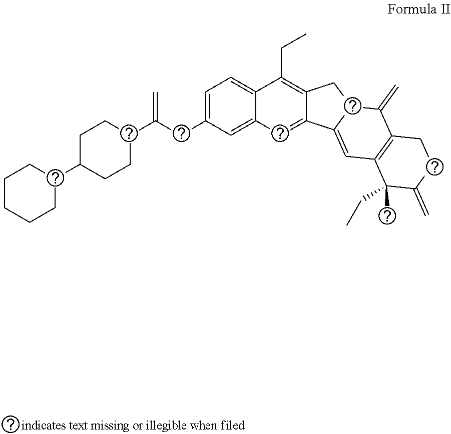 [SMILES: C=C(Cc1ccc2c(CC)c3c(cc2c1)C1=CC2=C(CCC(=C)[C@]2(C)CC)C(=C)C1C3)C1CCC(C2CCCCC2)CC1]

In another aspect it is provided a camptothecin derivative having the chemical structure of Formula III:

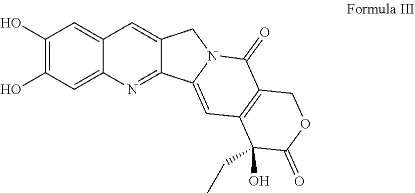 [SMILES: CC[C@@]1(O)C(=O)OCc2c1cc1n(c2=O)Cc2cc3cc(O)c(O)cc3nc2-1]

In yet another aspect it is provided a camptothecin derivative having the chemical structure of Formula IV:

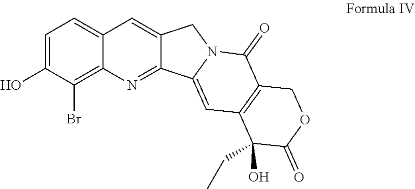 [SMILES: CC[C@@]1(O)C(=O)OCc2c1cc1n(c2=O)Cc2cc3ccc(O)c(Br)c3nc2-1]

In yet another aspect it is provided a camptothecin derivative having the chemical structure of Formula V:

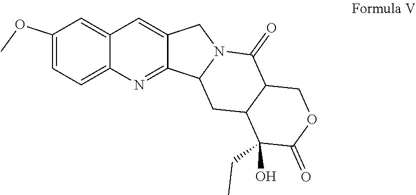 [SMILES: CC[C@@]1(O)C(=O)OCC2C(=O)N3Cc4cc5cc(OC)ccc5nc4C3CC21]

In yet another aspect it is provided a camptothecin derivative having the chemical structure of Formula VI:

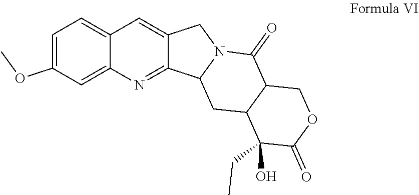 [SMILES: CC[C@@]1(O)C(=O)OCC2C(=O)N3Cc4cc5ccc(OC)cc5nc4C3CC21]

In a further aspect it is provided a camptothecin derivative having the chemical structure of Formula VII:

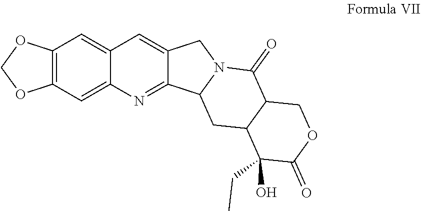 [SMILES: CC[C@@]1(O)C(=O)OCC2C(=O)N3Cc4cc5cc6c(cc5nc4C3CC21)OCO6]

Furthermore it is provide a pharmaceutical composition comprising an effective amount of the MIA derivative as described herewith. In one aspect the pharmaceutical composition may comprise an effective amount of 12-[(dimethylamino)methyl]-11-hydroxycamptothecin (topotecan-11), 7-ethyl-11-[4-(1-piperidino)-1-piperidino]carbonyloxycamptothecin (irinotecan-11), 10,11-dihydroxycamptothecin, 12-bromo-11-hydroxycamptothecin, 10-hydroxy-11-methoxycamptothecin or 11-hydroxy-10-methoxycamptothecin. In another aspect the pharmaceutical composition may comprise the camptothecin derivative of any one of Formula I, II, III, IV, V, VI or VII.

In a further aspect it is also provided a method of treating cancer in a subject, comprising administering to the subject a therapeutically effective amount of the camptothecin derivative as described herewith. Furthermore, a method of treating cancer in a subject is provided, the method comprising administering to the subject a therapeutically effective amount of the pharmaceutical composition as described herewith.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows the oxidation of camptothecin (CPT) and its analogues. Oxidation of CPT is central in the semi-synthesis of a variety of CPT-derived drugs such as irinotecan and topotecan. TDC, tryptophan decarboxylase; CYP450, cytochrome P450 enzyme from C. acuminata.

FIG. 2 shows camptothecin oxidation by Ca32229 (A) and Ca32236 (B): Extracted ion chromatograms from LC-MS analysis showing the in vivo conversion of CPT to 10HCPT (2.44 min, A) and 11HCPT (2.42 min, B) by Ca32236 and Ca32229, respectively. (C): NMR spectrum of hydroxylated products with the 1H NMR spectrum of 10HCPT standard showing the aromatic protons of ring A and H-14 (top, 7.20-8.20 ppm), and ID-TOC SY (50 ms spin-lock time) NMR spectra of aromatic protons on ring A of 10HCPT produced by Ca32236 (middle) and of 11HCPT produced by Ca32229 (bottom). *H-14 peak of 10HCPT is not shown in the 1D-TOC SY spectra as there is no correlation between H-14 and aromatic protons of ring A. CPT: camptothecin; HCPT: hydroxy-CPT; ECPT: ethyl-CPT; EV: empty vector (negative control).

FIG. 3 shows reaction schemes and LC-MS analysis of the production of camptothecin (CPT) analogues using CPT hydroxylases. FIG. 3A showns chemoenzymatic synthesis of topotecan (4) (Hycamtin®) and topotecan-11 (12-[(dimethylamino)methyl]-11-hydroxycamptothecin) (9) from CPT (1). FIG. 3B chemoenzymatic synthesis of irinotecan (3) (Camptosar®) and irinotecan-11 (7-ethyl-11-[4-(1-piperidino)-1-piperidino]carbonyloxy-CPT) (10) from 7-ethyl-CPT (6). Each LC-MS analysis panel include chromatograms for standard (top row), chemoenzymatic product (second row), enzymatic product (third row), and starting material (fourth row).

FIG. 4 shows identification of camptothecin (CPT) oxidative enzyme candidates. A, Abundance of CPT and 10-HCPT in different C. acuminata organs with error bars representing standard deviations (n=3). B. Self-organizing map code plot showing the nodes from where candidate genes were picked. C. Relative abundance of CYP450 candidates in different C. acuminata organs (colour scale: white to black shades correspond to low to high abundance levels).

FIG. 5 shows sequence analysis of CYP450 candidates. A. Unrooted neighbour-joining phylogenetic tree for CYP450 candidates from this study and previously reported CYP450s from C. acuminata and other organisms. Bootstrap frequencies for each Glade were based on 1000 iterations. Abbreviations and GenBank accession numbers for each protein are provided in the Material and Methods. B. Relative abundance of Ca32236 homologues in different organs. C. Alignment of Ca32229, Ca32245 and Ca32236.

FIG. 6 shows protein expression and in vitro assays of CYP450s. A. Western blot showing the expression of Ca32229 and 32236 in Saccharomyces cerevisiae harbouring pESC-Leu2d::CPR (EV: empty vector), pESC-Leu2d::32229/CPR and pESC-Leu2d::32236/CPR. Protein expression was induced by adding galactose. Recombinant P450 proteins were detected using a-FLAG antibodies. B. In vitro assays of total microsomal protein extracts of S. cerevisiae harbouring Ca32236 (left) and Ca32229 (right) with CPT.

FIG. 7 shows ¹H NMR spectra products from in vivo assay of CaCYP32236/CPR with camptothecin (CPT) producing 10HCPT (A), and with 7-ethyl-CPT as substrate producing 7-ethyl-10HCPT (B). ¹³C NMR spectra of 10HCPT (C) and 7-ethyl-10HCPT (D).

FIG. 8 shows ¹H NMR spectra of products from in vivo assay of Ca32229/CPR with camptothecin (CPT) as substrate producing 11HCPT (A), and with 7-ethyl-CPT as substrate producing 7-ethyl-11HCPT (B). ¹³C NMR spectra of 11HCPT (C) and 7-ethyl-11HCPT (D).

FIG. 9 shows substrate specificity of camptothecin hydroxylases (CPTHs), Ca32236 (CPT 10-hydroxylase) and Ca32229 (CPT 11-hydroxylase). Substrates from different subgroups of monoterpenoid indole alkaloids (MIA) include simple secoiridoid (secologanin), central precursors of MIA biosynthetic pathway (strictosidine, strictosamide), heteroyohimbanes (ajmalicine, tetrahydroalstonine), yohimbane (yohimbine), ajmalan (ajmaline), β-carbohne (harmalol, harmaline), and CPT and CPT analogues (10HCPT, 11HCPT, 7-ethyl-CPT, 9-amino-CPT, 9-nitro-CPT). Only CPT, 7-ethyl-CPT, 10HCPT, and 9-amino-CPT, as well as evodiamine were accepted as substrates with different conversion rates. Numbers in brackets are conversion rates, represented as [Ca32236 rate/Ca32229 rate] and [-] for non-detected rates.

FIG. 10 shows oxidation of 7-ethyl-CPT, 10HCPT and 11HCPT by Ca32229 and Ca32236. Extracted ion chromatograms showing the in vivo activity of Ca32236 (A) and Ca32229 (B) with 7-ethyl-CPT. CPT: camptothecin; HCPT: hydroxy-CPT; ECPT: ethyl-CPT; EV: empty vector (negative control). 10-HCPT can be further oxidized by Ca32229 (C) but not Ca32236 (D).

FIG. 11 shows ¹NMR spectrum of products from in vivo assay of Ca32229/CPR with 10HCPT as substrate producing 10,11-dihydroxy-CPT.

FIG. 12 shows oxidation of 9-amino-CPT by Ca32229 produces 9-amino-11HCPT, and Ca32236 to produce 9-amino-10HCPT. Extracted ion chromatograms showing the in vivo activity of Ca32236 and Ca32229. 9-Amino-CPT: 9-aminocamptothecin; EV: empty vector (negative control). The hydroxylation positions were speculated based on the regio-specificity of Ca32229 and Ca32236 toward other substrates of the same scaffold.

FIG. 13 shows chemoenzymatic production of topotecan (A) and topotecan-11 (12-[(dimethyl amino)methyl]-11HCPT) (B).

FIG. 14 shows ¹H NMR spectra of chemoenzymatic reaction products topotecan-11 (12-[(dimethylamino)methyl]-11HCPT) (A) and irinotecan-11 (7-ethyl-11-[4-(1-piperidino)-1-piperidino]carbonyloxyCPT) (B). ¹³C NMR spectra of topotecan-11 (C) and irinotecan-11 (D).

FIG. 15 shows chemoenzymatic production of irinotecan (A) and irinotecan-11 (7-ethyl-11-[4-(1-piperidino)-1-piperidino]carbonyloxyCPT) (B).

FIG. 16 shows chemoenzymatic production of brominated HCPTs using CPT 10-hydroxylase (A) and CPT 11-hydroxylase (B) as biocatalysts.

FIG. 17 shows ¹H NMR spectra of bromination reaction of 10HCPT as substrate producing 9-bromo-10HCPT (A), and of 11HCPT as substrate producing 12-bromo-11HCPT (B). ¹³C NMR spectra of 9-bromo-10HCPT (C) and 12-bromo-11HCPT (D).

FIG. 18 shows 1D-TOCSY NMR spectra of brominated products of 10HCPT and 11HCPT.

FIG. 19 depicts hydroxylated camptothecinoids and camptothecin (CPT) derivatives produced by chemoenzymatic reactions of the present disclosure. Disclosed compounds depicted in the right panel include 10-hydroxy-CPT (2), 11-hydroxy-CPT (5), 7-ethyl-10-hydroxy-CPT (7), 7-ethyl-11-hydroxy-CPT (8), 10,11-dihydroxy-CPT (12), 9-amino-10-hydroxy-CPT (18), 9-amino-11-hydroxy-CPT (19), topotecan (4), 12-[(dimethylamino)methyl]-11-hydroxy-CPT (9), 9-bromo-10-hydroxy-CPT (15), 12-bromo-11-hydroxy-CPT (17), irinotecan-11 (10), and irinotecan (3).

FIG. 20 shows production of (A) 10-hydroxycamptothecin and (B) 11-hydroxycamptothecin in Nicotiana benthamiana.

FIG. 21 shows chemoenzymatic production of hydroxylated evodiamine using CPT 11-hydroxylase (left) and CPT 10-hydroxylase (right) as biocatalysts.

### DETAILED DESCRIPTION

The following description is of a preferred embodiment.

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a use or method, denotes that additional elements and/or method steps may be present, but that these additions do not materially affect the manner in which the recited method or use functions. The term “consisting of” when used herein in connection with a use or method, excludes the presence of additional elements and/or method steps. A use or method described herein as comprising certain elements and/or steps may also, in certain embodiments, consist essentially of those elements and/or steps, and in other embodiments consist of those elements and/or steps, whether or not these embodiments are specifically referred to. In addition, the use of the singular includes the plural, and “or” means “and/or” unless otherwise stated. The term “plurality” as used herein means more than one, for example, two or more, three or more, four or more, and the like. Unless otherwise defined herein, all technical and scientific terms used herein have the same meaning as commonly understood by one of ordinary skill in the art. As used herein, the term “about” refers to an approximately +/−10% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

The term “recombinant” may mean that something has been recombined, so that when made in reference to a nucleic acid construct the term may refer to a molecule that is comprised of nucleic acid sequences that are joined together or produced by means of molecular biological technique. When made in reference to a protein or polypeptide, the term “recombinant” may refer to a protein or polypeptide molecule that may be expressed using a recombinant nucleic acid construct created by means of molecular biological techniques.

The term “heterologous” in reference to a nucleic acid or protein may be a molecule that has been manipulated by human intervention so that it may be located in a place other than the place in which it is naturally found. For example, a nucleic acid sequence from one species may be introduced into the genome of another species, or a nucleic acid sequence from one genomic locus may be moved to another genomic or extrachromosomal locus in the same species.

A “protein,” “peptide” or “polypeptide” is any chain of two or more amino acids, including naturally occurring or non-naturally occurring amino acids or amino acid analogues, regardless of post-translational modification (e.g., glycosylation or phosphorylation). An “amino acid sequence”, “polypeptide”, “peptide” or “protein” of the disclosure may include peptides or proteins that have abnormal linkages, cross links and end caps, non-peptidyl bonds or alternative modifying groups. Such modified peptides may be also within the scope of the invention.

A “substantially identical” sequence may be an amino acid or nucleotide sequence that may differ from a reference sequence by one or more conservative substitutions, or by or by one or more non-conservative substitutions, deletions, or insertions located at positions of the sequence that do not destroy the biological function of the amino acid or nucleic acid molecule. Such a sequence may be any value from 40% to 99%, or more generally at least 50%, 55%, 60%, 65%, 70%, 75%, 80%, 85%, 90%, or 95%, or as much as 96%, 97%, 98%, or 99% identical when optimally aligned at the amino acid or nucleotide level to the sequence used for comparison.

“Derived from” is used to mean taken, obtained, received, traced, replicated or descended from a source (chemical and/or biological). A derivative may be produced by chemical or biological manipulation (including, but not limited to, substitution, addition, insertion, deletion, extraction, isolation, mutation and replication) of the original source.

### Cytochrome P450 Monooxygenase Enzymes

The present description relates to cytochrome P450 monooxygenase enzymes capable of oxidizing a monoterpenoid indole alkaloid (MIA), wherein the scaffold of the MIA may comprises quinoline moiety or indole moiety. For example the quinoline moiety comprising compound might be a camptothecinoid and the indole moiety comprising compound may be a evodiaminoid or ellipticinoid. The cytochrome P450 monooxygenase enzymes of the current disclosure are capable of regio-specifically oxidizing the MIA to produce a hydroxylated MIA. For example the cytochrome P450 monooxygenase enzymes are capable of producing hydroxylated campothecinoids (hydroxycamptothecinoids), hydroxylated evodiaminoids (hydroxyevodiaminoid) or hydroxylated ellipticinoids (hyroxyellipticinoid).

In the contect of the present disclosure, the term “hydroxylation” refers to an oxidation reaction in which a carbon-hydrogen (C—H) bond oxidizes into carbon-hydroxyl (C—OH) bond. Accordingly, in some instances the terms oxidation or hydroxylation might be used interchangeably.

Cytochrome P450 enzyme (CYPs) (also referred to as ‘cytochrome P450 monooxygenase’, ‘CYP450’, ‘cytochrome P450 enzymes’, ‘P540 enzymes’, ‘cytochrome P450’, ‘P450) are a superfamily of enzymes containing heme (or haem) as a cofactor that functions as monooxygenases. Cytochrome P450 enzymes use heme to oxidize substrates, typically using protons from donor NAD(P)H to split oxygen such that a single oxygen atom can be added to a substrate. As further described herein, the cytochrome P450 monooxygenase may be a hydroxylase. A hydroxylase refers to any enzyme which adds a hydroxyl group to an organic substrate. The cytochrome P450 monooxygenase enzymes described herewith may also be referred to as “oxidative enzymes”, ‘hydroxylase”, “camptothecinoid hydroxylase”, “camptothecin hydroxylase” or “CPT X-hydroxylase” (“CPTXH”), wherein X denotes the position of hydroxylation within a MIA substrate, such for example camptothecinoid, evodiaminioid and ellipticinoid substrates. X may for example be 1, 4, 5, 7, 9, 10, 11, 12, 14, 18, 19 or 22 (see table 1). Accordingly, the CPT X hydroxylase may be CPT1H, CPT4H, CPT5H, CPT7H, CPT9H, CPT10H, CPT11H, CPT12H, CPT14H, CPT18H, CPT19H or CPT22H.

The CPTXH enzyme may have an amino acid sequence that has about 70, 75, 80, 85, 87, 90, 91, 92, 93 94, 95, 96, 97, 98, 99, 100% or any amount therebetween, sequence identity, or sequence similarity, with any one amino acid sequence of SEQ ID NO: 3, 4, 8, 9, 10, 14, 15, 16, 18, 20, 22, 24, 26, 28 or 30, or an active fragment or a degenerative variant thereof, wherein the enzyme has hydroxylase or MIA hydroxylase activity, as described herewith. The amino acid may be a purified amino acid, such as a purified protein or enzyme.

The CPTXH enzyme may further be encoded by a nucleic acid that has about 70, 75, 80, 85, 87, 90, 91, 92, 93 94, 95, 96, 97, 98, 99, 100% or any amount therebetween, sequence identity, or sequence similarity, with the nucleotide sequence according to SEQ ID NO: 1, 2, 5, 6, 7, 11, 12, 13, 17, 19, 21, 23, 25, 27, or 29. The nucleic acid may be a purified nucleic acid.

An “isolated” or “purified” protein or nucleic acid molecule is substantially or essentially free from components that normally accompany or interact with the protein or nucleic acid molecule as found in its naturally occurring environment. Thus, an isolated or purified protein or nucleic acid molecule is substantially free of other cellular material, or culture medium when produced by recombinant techniques, or substantially free of chemical precursors or other chemicals when chemically synthesized.

The CPTXH may be fused to a tag protein or peptide to form a CPTXH-tag fusion protein.

Although the cytochrome P450 monooxygenase enzymes as described herewith may also be referred to “camptothecinoid hydroxylase” or “camptothecin hydroxylase”, it has been found that the cytochrome P450 monooxygenase enzymes are capable of oxidizing other substrates than camptothecinoids or camptothecin, as described below. Therefore the expressions “camptothecinoid hydroxylase” or “camptothecin hydroxylase” are not limited to enzymes that only catalyze the oxidation of camptothecinoids or camptothecin, but it will be understood that other substrates such for example ellipticinoids or evodiaminoids, may be oxidized by the enzymes, as described below.

The cytochrome P450 monooxygenase enzyme may catalyze the oxidation of carbon at positions in the quinoline moiety or indole moiety of the MIA substrate, but may also hydroxylate other positions within the compound. Possible positions of hydroxylation are indicated in Table 1.

For example the cytochrome P450 monooxygenase enzyme may catalyze the oxidation of carbon C5, C6 or C7 of the quinoline moiety in the MIA or the cytochrome P450 monooxygenase enzyme may catalyze the oxidation of C4, C5 or C6 of the indole moiety in the MIA. Corresponding positions in camptothecinoid, evodiaminioid and ellipticinoid substrates are indicated in Table 1. For example the cytochrome P450 monooxygenase enzyme may catalyze the oxidation of positions C9, C10 or C11 in camptothecinoid or evodiaminioid substrates or positions C10, C9 or C8 in ellipticinoid substrates.

TABLE 1
Numbering of Carbon (C) atom in MIA
that might be hydroxylated by CYP450

| Quinoline | Indole | | | | | | |
|---|---|---|---|---|---|---|---|
| Moiety | Moiety | Camptothecinoid | | Evodiaminioid | | Ellipticinoid | |
| — | — | — | | — | | C1 | (D) |
| — | — | — | | — | | C3 | (D) |
| — | — | — | | — | | C4 | (D) |
| — | — | — | | C1 | (E) | — | |
| — | — | — | | C2 | (E) | — | |
| — | — | — | | C3 | (E) | — | |
| — | — | — | | C4 | (E) | — | |
| — | — | C4 | (C) | — | | | |
| — | — | C5 | (C) | — | | — | |
| C1 | — | C1 | (B) | — | | | |
| C4 | — | C7 | (B) | — | | — | |
| — | — | — | | C7 | (C) | — | |
| — | — | — | | C8 | (C) | — | |
| C5 | C4 | C9 | (A) | C9 | (A) | C10 | (A) |
| C6 | C5 | C10 | (A) | C10 | (A) | C9 | (A) |
| C7 | C6 | C11 | (A) | C11 | (A) | C8 | (A) |
| C8 | C7 | C12 | (A) | C12 | (A) | C7 | (A) |

| | | | | | |
|---|---|---|---|---|---|
| — | — | C14 | (D) | — | — |

| | | | | | |
|---|---|---|---|---|---|
| — | — | — | C15 | (D) | — |

| | | | | | |
|---|---|---|---|---|---|
| — | — | C18 | (E) | — | — |
| — | — | C19 | (E) | — | — |
| — | — | C22 | (E) | — | — |

| | | | | | |
|---|---|---|---|---|---|
| — | — | — | — | C12 | (C) |
| — | — | — | — | C13 | (C) |

*Letters in brackets indicate the ring letter of compound

The cytochrome P450 monooxygenase may be a plant cytochrome P450 monooxygenase. For example the cytochrome P450 monooxygenase may be derived from a plant such for example from Camptotheca spp., Ophiorrhiza spp., Notapodytes spp. and members of Nothapodytes, Ophiorrhiza, Chonemorpha, Apodytes, Merillodendron, Dysoxylum, Tabernaemontana, Codiocarpus, Pyrenacantha, Mostuea, or Iodes. A non-limiting example of a cytochrome P450 monooxygenase as described herewith are cytochrome P450 monooxygenase enzymes derived from Camptotheca acuminata, Ophiorrhiza pumila or Nothapodytes nimmoniana.

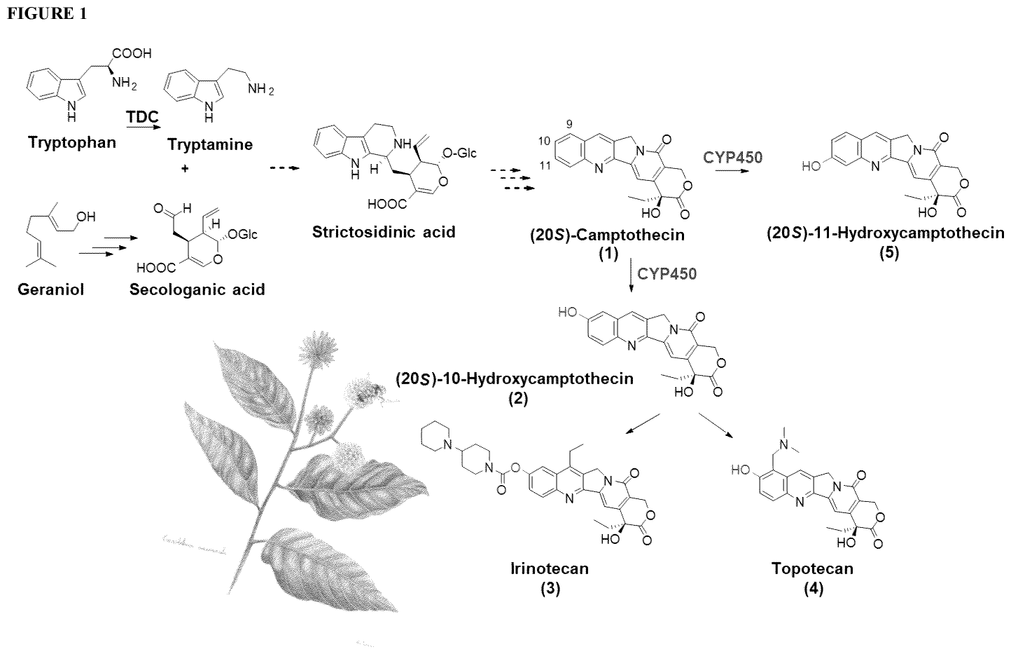

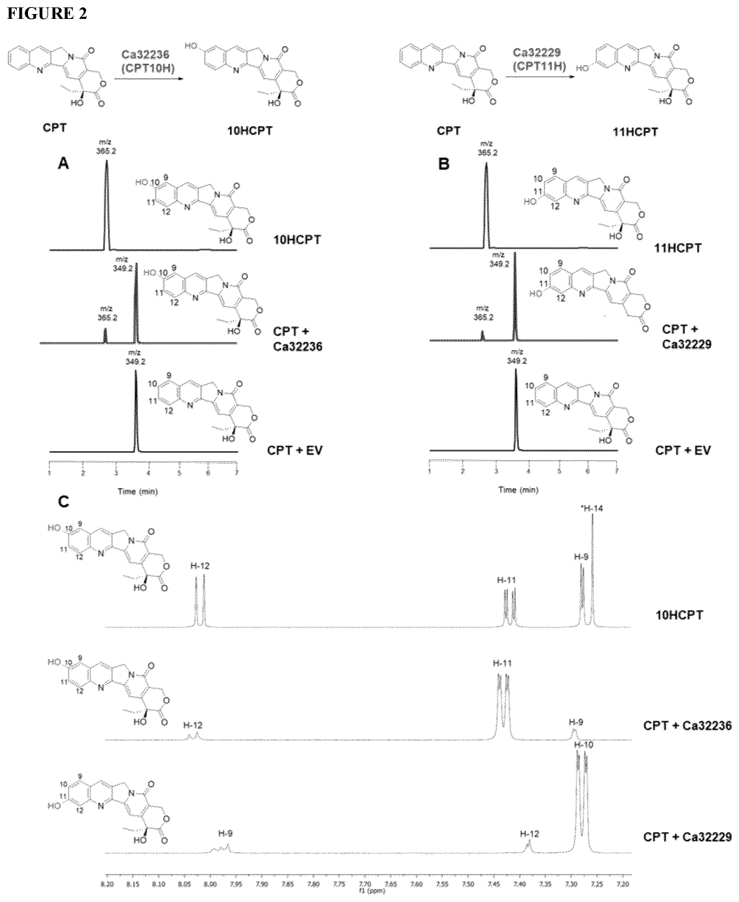

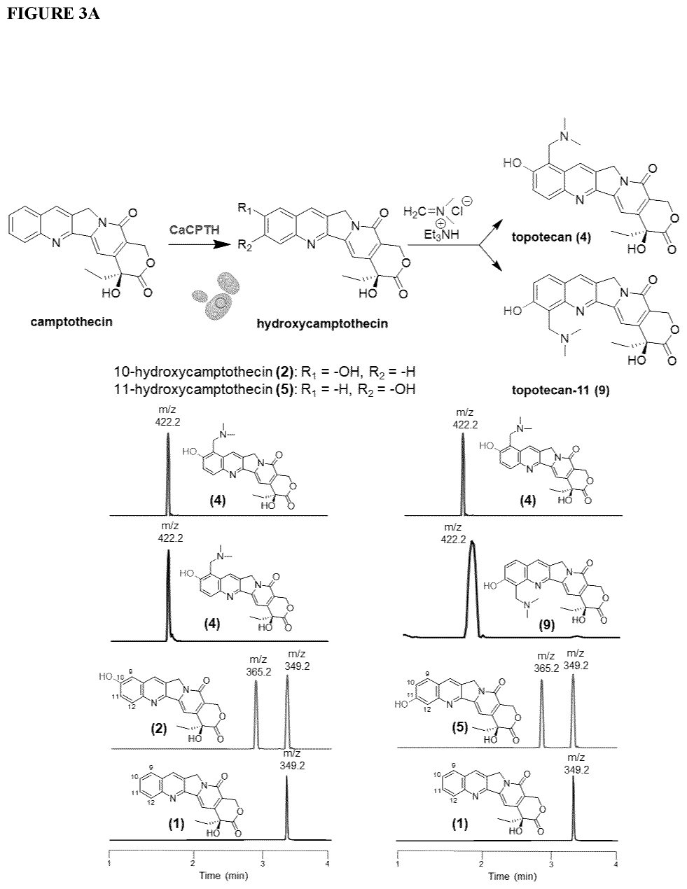

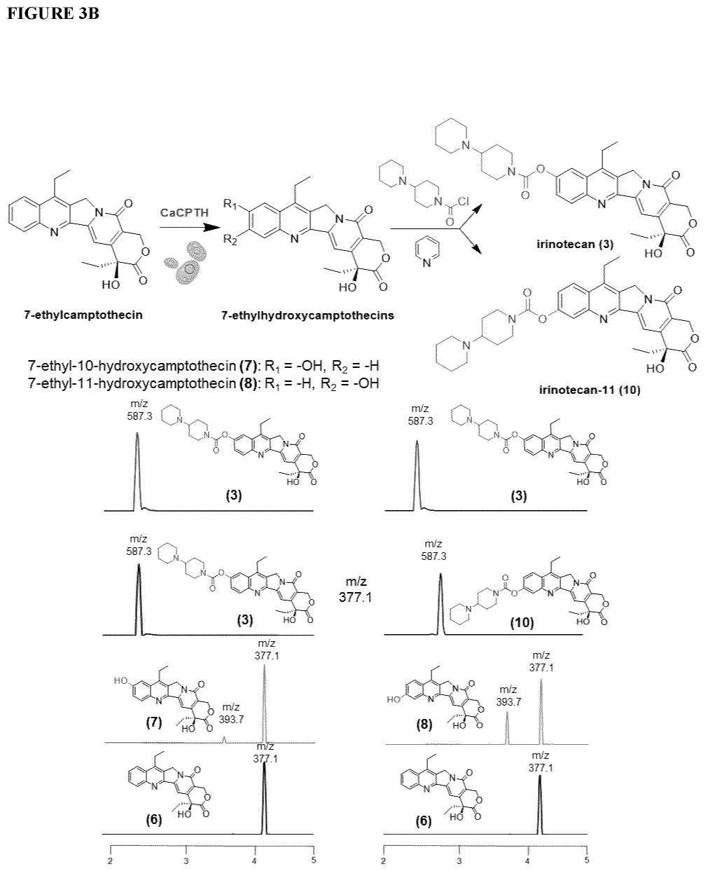

For example, in one embodiment the cytochrome P450 monooxygenase (alternatively referred to as a hydroxylase or camptothecin hydroxylase) may catalyze the oxidation of camptothecin to 10-hydroxycamptothecin (see FIGS. 1 and 2, Example 4). In another embodiment, the cytochrome P450 monooxygenase (or a hydroxylase or camptothecin hydroxylase) may catalyze the oxidation of camptothecin to 11-hydroxycamptothecin (see FIG. 2, Example 4). As further embodied, the cytochrome P450 monooxygenase (or a hydroxylase or camptothecin hydroxlase) may catalyze the oxidation of 7-ethylcamptothecin to 7-ethyl-10-hydroxycamptothecin or 7-ethyl-11-hydroxycamptothecin (see FIG. 3B Example 4). The activity of the cytochrome P450 monooxygenase is not limited by these examples and may encompass any appropriate MIA substrate for oxidation or hydroxylation.

The cytochrome P450 monooxygenase may yield conversion of the MIA substrate (for example camptothecinoid) to the hydroxylated MIA (for example hydroxylated camptothecinoid) at an efficiency of about 10-12 mg hydroxylated MIA per litre. The hydroxylated MIA (for example hydroxylated camptothecinoid) may be isolated or recovered at a yield of approximately 7-8 mg dried product per litre. The cytochrome P450 monooxygenase may yield conversion of the MIA such as camptothecinoid to the hydroxylated MIA such as hydroxylated camptothecinoid at an improved efficiency rate compared to traditional chemical conversion.

CPT 9-hydroxylase (CPT9H)

The cytochrome P450 monooxygenase as described herewith may be “CPT 9-hydroxylase” (CPT9H). CPT9H may oxidize C5 of the quinoline moiety of the MIA substrate or C4 of the indole moiety of the MIA substrate.

Without wishing to be bound by theory, it is believed that CPT9H oxidizes C5 of the quinoline moiety of the MIA substrate or C4 of the indole moiety of the MIA substrate, based on the following: i) 9-methoxycamptothecin is a natural product that has been isolated from the tender roots and stem of C. acuminata. The O-methyltransferase enzyme requires 9-hydroxycamptothecin as substrate to produce 9-methoxycamptothecin (see Sun et al. Natural Product Research, Volume 35, 2021). ii) It has been found that CPT9H from C. acuminata shares high sequence homology/identity (about 80%) with CPT10H from C. acuminate. iii) When CPT9H is contacted with a camptothecinoid substrate the retention time of the resulting hydroxylated camptothecinoid product differs from the retention time of the corresponding 10-hydroxycamptothecinoid or 11-hydroxycamptothecinoid (data not shown). It is therefore soundly predicted that CPT9H hydroxylates a camptothecinoid substrate at position C9 to produce a 9-hydroxycamptothecinoid and therefore CPT9H may oxidize C5 of the quinoline moiety of the MIA substrate or C4 of the indole moiety of the MIA.

The CPT9H enzyme may be a plant CPT 9-hydroxylase. A non-limiting example of CPT9H is CPT 9-hydroxylase from Camptotheca acuminata, CPT 9-hydroxylase from Ophiorrhiza pumila or CPT 9-hydroxylase from Nothapodytes nimmoniana.

Accordingly, the cytochrome P450 monooxygenase may be CPT9H from Camptotheca acuminata, Ophiorrhiza pumila, Nothapodytes nimmoniana or any homologous or orthologous hydroxylase with similar function and substrate recognition.

The CPT9H enzyme may have an amino acid sequences that has about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 26, or an active fragment or a degenerative variant thereof, wherein the enzyme has hydroxylase or MIA hydroxylase activity, as described herewith.

The CPT9H enzyme may further be encoded by a nucleic acid that has about 70, 75, 80, 85, 87, 90, 91, 92, 93 94, 95, 96, 97, 98, 99, 100% or any amount therebetween, sequence identity, or sequence similarity, with the nucleotide sequence according to SEQ ID NO: 25.

CPT 10-hydroxylase (CPT10H)

The cytochrome P450 monooxygenase as described herewith may be “CPT 10-hydroxylase” (CPT10H). CPT10H may oxidize C6 of the quinoline moiety of the MIA substrate or C5 of the indole moiety of the MIA substrate.

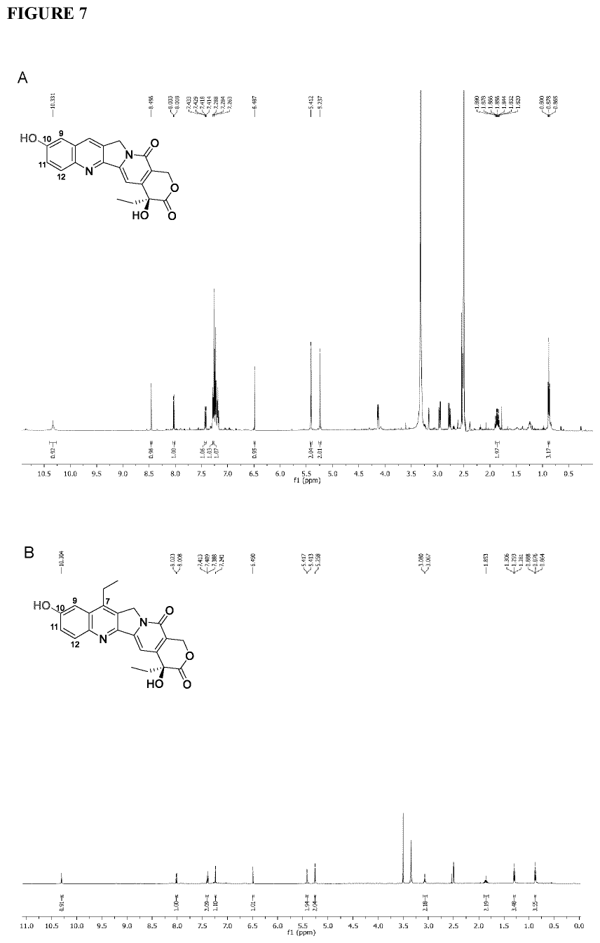

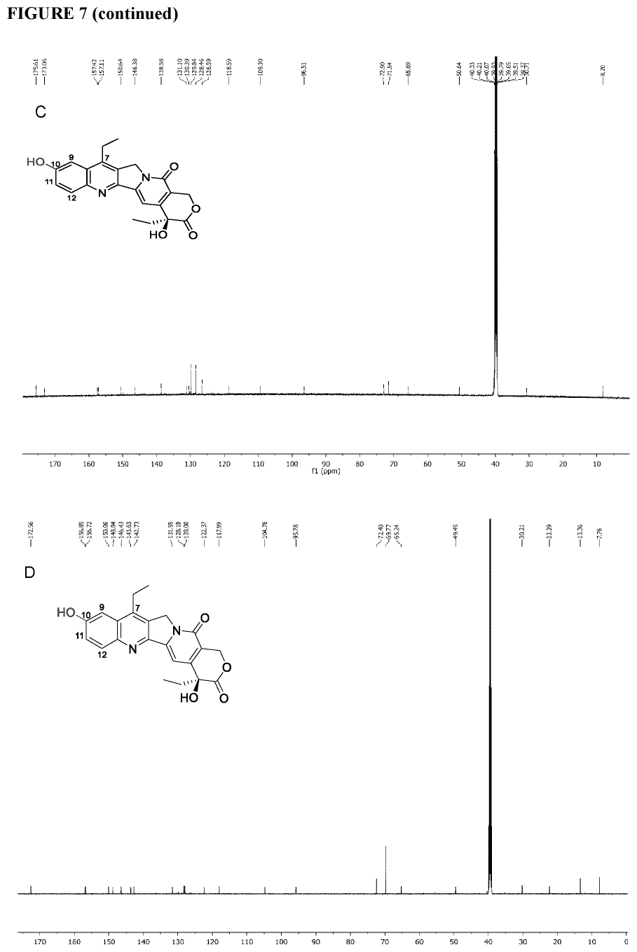

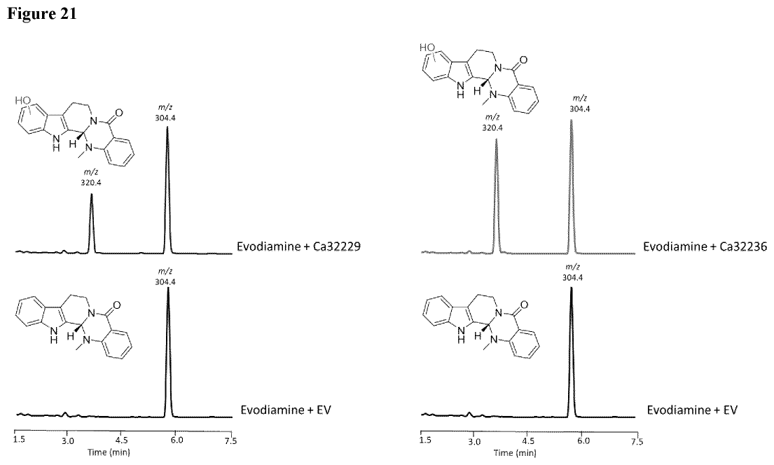

As shown in Example 4 and FIGS. 7, 10A and 12, a cytochrome P450 monooxygenase (CPT10H) as described herewith when contacted with monoterpenoid indole alkaloid (MIA) substrates, wherein the scaffold of the MIA comprises quinoline produced a hydroxylated monoterpenoid indole alkaloid (HMIA), wherein C6 of the quinoline moiety (equivalent to C10 of Camptothecinoid) is hydroxylated. As further shown in FIG. 21 (right column), when CPT10H was contacted with a MIA substrates, wherein the scaffold of the MIA comprises indole (for example an evodiaminoid) a hydroxylated MIA was produced. Without wishing to be bound by theory, it is believed that the hydroxylated MIA is 10-hydroxyl evodiaminoid.

The CPT10H enzyme may be a plant CPT 10-hydroxylase. A non-limiting example of CPT10H is CPT 10-hydroxylase from Camptotheca acuminata (also referred to as “CaCYP32236” or “Ca32236”) CPT 10-hydroxylase from Ophiorrhiza pumila or CPT 10-hydroxylase from Nothapodytes nimmoniana. Accordingly, the cytochrome P450 monooxygenase may be CPT10H from Camptotheca acuminata, Ophiorrhiza pumila, Nothapodytes nimmoniana or any homologous or orthologous hydroxylase with similar function and substrate recognition.

The CPT10H enzyme may have an amino acid sequences that has about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 3, 8, 9, 10, 18 or an active fragment or a degenerative variant thereof, wherein the enzyme has hydroxylase or MIA hydroxylase activity, as described herewith.

The CPT10H enzyme may further be encoded by a nucleic acid that has about 70, 75, 80, 85, 87, 90, 91, 92, 93 94, 95, 96, 97, 98, 99, 100% or any amount therebetween, sequence identity, or sequence similarity, with the nucleotide sequence according to SEQ ID NO: 1, 5, 6, 7, or 17.

CPT 11-hydroxylase (CPT11H)

The cytochrome P450 monooxygenase as described herewith may be “CPT 11-hydroxylase” (CPT11H). CPT11H may oxidize C7 of the quinoline moiety of the MIA substrate or C6 of the indole moiety of the MIA substrate.

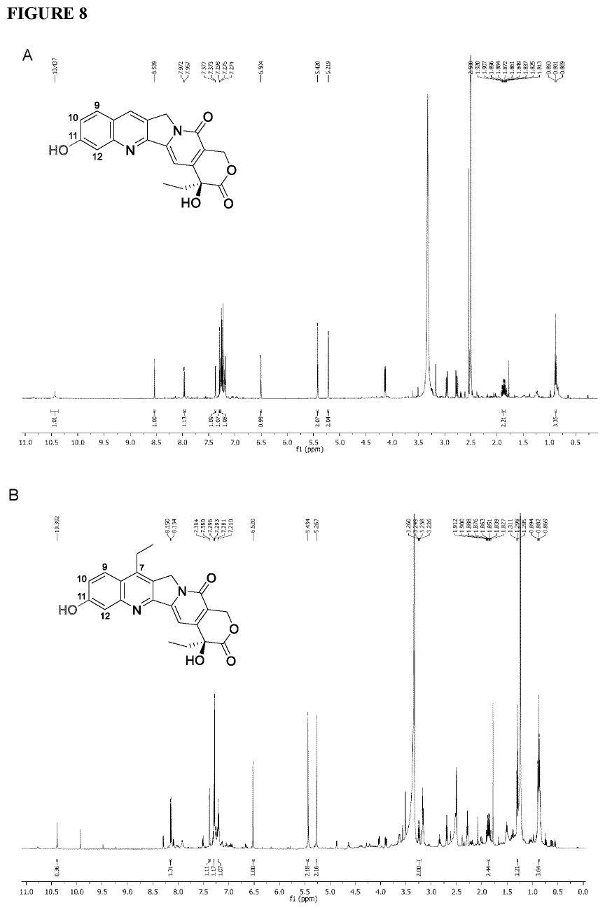

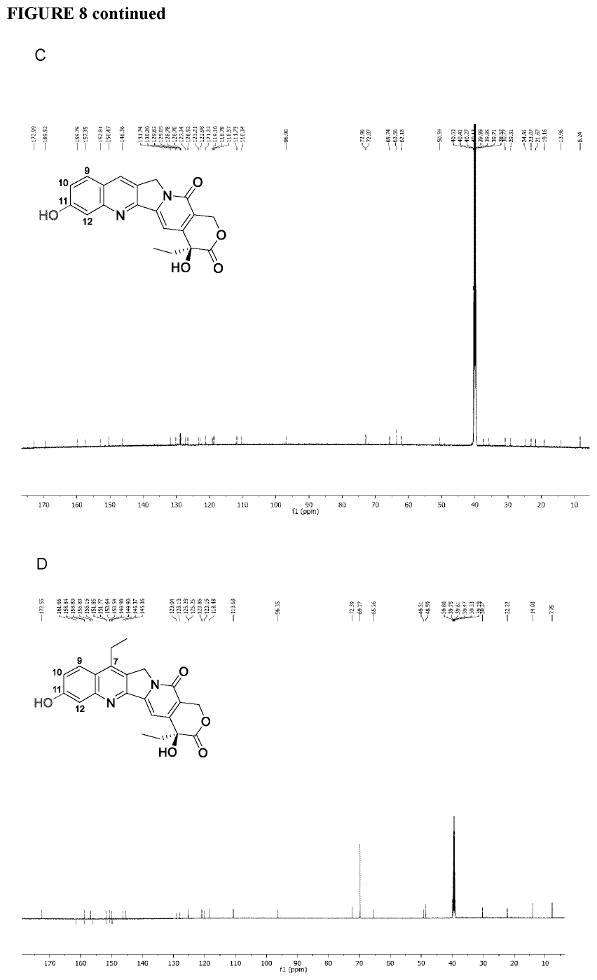

As shown in Example 4 and FIGS. 8, 10B, 10C and 12, a cytochrome P450 monooxygenase (CPT11H) as described herewith when contacted with a MIA, wherein the scaffold of the MIA comprises quinoline produced a hydroxylated monoterpenoid indole alkaloid (HMIA), wherein C7 of the quinoline moiety (equivalent to C11 of Camptothecinoid) is hydroxylated. As further shown in FIG. 21 (left column), when CPT11H was contacted with a MIA substrates, wherein the scaffold of the MIA comprises indole (for example an evodiaminoid) a hydroxylated MIA was produced. Without wishing to be bound by theory, it is belived that the hydroxylated MIA is 11-hydroxyl evodiaminoid.

The CPT11H enzyme may be a plant CPT 11-hydroxylase. A non-limiting example of CPT11H is CPT 11-hydroxylase from Camptotheca acuminata (also referred to as “CaCYP32229” or “Ca32229”), CPT 11-hydroxylase from Ophiorrhiza pumila or CPT 11-hydroxylase from Nothapodytes nimmoniana. Accordingly, the cytochrome P450 monooxygenase may be CPT11H from Camptotheca acuminata, Ophiorrhiza pumila, Nothapodytes nimmoniana or any homologous or orthologous hydroxylase with similar function and substrate recognition.

The CPT11H enzyme may have an amino acid sequences that has about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 4, 14, 15, 16, 20, or an active fragment or a degenerative variant thereof, wherein the enzyme has hydroxylase or MIA hydroxylase activity, as described herewith.

The CPT11H enzyme may further be encoded by a nucleic acid that has about 70, 75, 80, 85, 87, 90, 91, 92, 93 94, 95, 96, 97, 98, 99, 100% or any amount therebetween, sequence identity, or sequence similarity, with the nucleotide sequence according to SEQ ID NO: 2, 11, 12, 13, or 19.

“Homologous gene” or “homologs” refers to genes derived from a common ancestral gene, which are found in two species. Genes are considered homologs when their nucleotide sequences and/or their encoded protein sequences share substantial identity or similarity as defined below.

“Orthologous genes” or “orthologs” refers to homologous genes derived from a common ancestral gene and which are found in different species as a result of speciation. Genes found in different species are considered orthologs when their nucleotide sequences and/or their encoded protein sequences share substantial identity or similarity as defined below. Functions of orthologs are often highly conserved among species.

A degree of homology or similarity or identity between nucleic acid sequences is a function of the number of identical or matching nucleotides at positions shared by the nucleic acid sequences.

The terms “percent similarity”, “sequence similarity”, “percent identity”, or “sequence identity”, when referring to a particular sequence, are used for example as set forth in the University of Wisconsin GCG software program, or by manual alignment and visual inspection (see, e.g., Current Protocols in Molecular Biology, Ausubel et al., eds. 1995 supplement). Methods of alignment of sequences for comparison are well-known in the art. Optimal alignment of sequences for comparison can be conducted, using for example the algorithm of Smith & Waterman, (1981, Adv. Appl. Math. 2:482), by the alignment algorithm of Needleman & Wunsch, (1970, J. Mol. Biol. 48:443), by the search for similarity method of Pearson & Lipman, (1988, Proc. Natl. Acad. Sci. USA 85:2444), by computerized implementations of these algorithms (for example: GAP, BESTFIT, FASTA, and TFASTA in the Wisconsin Genetics Software Package, Genetics Computer Group (GCG), 575 Science Dr., Madison, Wis.).

An example of an algorithm suitable for determining percent sequence identity and sequence similarity are the BLAST and BLAST 2.0 algorithms, which are described in Altschul et al., (1977, Nuc. Acids Res. 25:3389-3402) and Altschul et al., (1990, J. Mol. Biol. 215:403-410), respectively. BLAST and BLAST 2.0 are used, with the parameters described herein, to determine percent sequence identity for the nucleic acids and proteins of the disclosure. For example the BLASTN program (for nucleotide sequences) may use as defaults a wordlength (W) of 11, an expectation (E) of 10, M=5, N=−4 and a comparison of both strands. For amino acid sequences, the BLASTP program may use as defaults a word length of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands. Software for performing BLAST analyses is publicly available through the National Center for Biotechnology Information (see URL: ncbi.nlm.nih.gov/).

A nucleic acid sequence or nucleotide sequence referred to in the present disclosure, may be “substantially homologous”, “substantially orthologous”, “substantially similar” or “substantially identical” to a sequence, or a compliment of the sequence if the nucleic acid sequence or nucleotide sequence hybridise to one or more than one nucleotide sequence or a compliment of the nucleic acid sequence or nucleotide sequence as defined herein under stringent hybridisation conditions. Sequences are “substantially homologous”, “substantially orthologous”, “substantially similar” “substantially identical” when at least about 70%, or between 70 to 100%, or any amount therebetween, for example 70, 72, 74, 76, 78, 80, 82, 84, 86, 88, 90, 91, 92,93, 94, 95, 96, 97, 98, 99, 100%, or any amount therebetween, of the nucleotides match over a defined length of the nucleotide sequence providing that such homologous sequences exhibit one or more than one of the properties of the sequence, or the encoded product as described herein.

The cytochrome P450 monooxygenase enzyme as described herewith, may be a purified cytochrome P450 monooxygenase enzyme.

The cytochrome P450 monooxygenase enzyme as described herewith, may further be a recombinant protein which is expressed in a host or host cell, therefore the present disclosure also provides a recombinant cytochrome P450 monooxygenase enzyme. The cytochrome P450 monooxygenase enzyme may further be modified compared to the native enzyme. For example the cytochrome P450 monooxygenase enzyme may be modified to include deletions, subsitutions or mutations, or the cytochrome P450 monooxygenase enzyme may be modified to be expressed as a fusion and/or chimeric protein. Accordingly, when referring to cytochrome P450 monooxygenase in this description, modified cytochrome P450 monooxygenase enzymes that are capable of regio-specifically oxidizing the MIA to produce a hydroxylated MIA as described herewith are also included.

For example, the modified cytochrome P450 monooxygenase enzyme may be a truncated enzyme (truncated CYP450′), wherein amino acid residues from the N-terminus, the C-terminus or both from the N-terminus and C-terminus may be deleted from the enzyme while still retaining its catalytic activity. For example, 1 to 100, or more amino acids may be removed from the N-terminus, the C-terminus or both from the N-terminus and C-terminus of the enzyme, while still retaining activity of oxidizing the MIA to produce a hydroxylated MIA.

Furthermore, the modified cytochrome P450 monooxygenase enzyme may be a chimeric cytochrome P450 monooxygenase enzyme (chimeric CYP450′) or fusion cytochrome P450 monooxygenase enzyme (fusion CYP450′). In the chimeric or fusion CYP450, heterologous peptides, proteins and/or protein fragments may be fused to the native CYP450 protein. Altenatively, portions of the native CYP450 protein may be replaced with heterologous peptides, proteins and/or protein fragments or portion of the heterologous protein. For example, the heterologous peptides, proteins and/or protein fragments or portion of the heterologous protein may be fused to the C-terminus, N-terminus or both the the N-terminus and C-terminus of the CYP450 enzyme, or the heterologous peptides, proteins and/or protein fragments or portion of the heterologous protein may be fused into the coding sequence of the CYP450 enzyme (internal fusion). For example the chimeric CYP450 protein may have i) a greater catalytic efficiency compared to the native CYP450 by altering the tertiary and quaternary structure of the CYP450 enzyme, ii) increased solubility compared to the native CYP450; iii) increased thermostability and stability over a wider pH range compared to the native CYP450; iv) increased enzyme activity compared to the native CYP450, and/or v) increased expression levels in and/or secretion level from a host or host cell compared to the native CYP450.

For example, the modified cytochrome P450 monooxygenase enzyme may include one or more than one protein tag and/or a cleavage site. The modified cytochrome P450 monooxygenase enzyme may also be referred to as fusion cytochrome P450 monooxygenase enzyme, wherein the fusion cytochrome P450 monooxygenase enzyme comprises the native cytochrome P450 monooxygenase enzyme fused to one or more than one tag or tag peptide. The one or more than one tag may be added to either end of cytochrome P450 monooxygenase enzyme, therefore the tag may be C-terminus, N-terminus specific or both C-terminus and N-terminus specific. The tag may also be inserted into the coding sequence of the cytochrome P450 monooxygenase enzyme (internal tag).

Protein and peptide (epitope) tags are well known within the art and are widely used in protein purification and protein detection (see for example Johnson M. Mater Methods 2012; 2:116, which is incorporated herewith by reference). For example, the cytochrome P450 monooxygenase of the current disclosure, may be tagged with an affinity tag, solubilization tag, chromatography tag, epitope tag, fluorescence tags. For example the protein tag may be selected from one or more of: Albumin-binding protein (ABP); Alkaline Phosphatase (AP); AU1 epitope; AU5 epitope; AviTag; Bacteriophage T7 epitope (T7-tag); Bacteriophage V5 epitope (V5-tag); Biotin-carboxy carrier protein (BCCP); Bluetongue virus tag (B-tag); single-domain camelid antibody (C-tag); Calmodulin binding peptide (CBP or Calmodulin-tag); Chloramphenicol Acetyl Transferase (CAT); Cellulose binding domain (CBP); Chitin binding domain (CBD); Choline-binding domain (CBD); Dihydrofolate reductase (DHFR); DogTag; E2 epitope; E-tag; FLAG epitope (FLAG-tag); c-myc epitope (c-myc-tag) Galactose-binding protein (GBP); Green fluorescent protein (GFP); Glu-Glu (EE-tag); Glutathione S-transferase (GST); Human influenza hemagglutinin (HA); HaloTag™; Alternating histidine and glutamine tags (HQ tag); Alternating histidine and asparagine tags (HN tag); Histidine affinity tag (HAT); Horseradish Peroxidase (HRP); HSV epitope; Isopeptag (Isopep-tag); Ketosteroid isomerase (KSI); KT3 epitope; LacZ; Luciferase; Maltose-binding protein (MBP); Myc epitope (Myc-tag); NE-tag; NusA; PDZ domain; PDZ ligand; Polyarginine (Arg-tag); Polyaspartate (Asp-tag); Polycysteine (Cys-tag); Polyglutamate (Glu-tag); Polyhistidine (His-tag); Polyphenylalanine (Phe-tag); Profinity eXact; Protein C; Rho1D4-tag; Si-tag; S-tag; Softag 1; Softag 3; SnoopTagJr; SnoopTag; Spot-tag; SpyTag (Spy-tag); Streptavadin-binding peptide (SBP); Staphylococcal protein A (Protein A); Staphylococcal protein G (Protein G); Strep-tag; Streptavadin (SBP-tag); Strep-tag II; Sdy-tag; Small Ubiquitin-like Modifier (SUMO); Tandem Affinity Purification (TAP); T7 epitope; tetracysteine tag (TC tag); Thioredoxin (Trx); TrpE; Ty tag; Ubiquitin; Universal; V5 tag; VSV-G or VSV-tag; and Xpress tag. For example, in one embodiment the modified cytochrome P450 monooxygenase enzyme may be a fusion cytochrome P450 monooxygenase enzyme comprising cytochrome P450 monooxygenase enzyme as described herwith fused to a FLAG epitope (FLAG-tag) or c-myc epitope (c-myc-tag).

Therefore in accordance with a further embodiment, there is provided a vector including the nucleic acid described herein. The vector may also include a heterologous nucleic acid sequence is selected from one or more of the following: a protein tag; and a cleavage site.

The present disclosure further provides vector or construct comprising a nucleic acid comprising a nucleotide sequence encoding the cytochrome P450 monooxygenase enzyme of the present disclosure. The vector may be suitable as an expression vector, cloning vector, or integrative vector.

The term “construct”, “vector” or “expression vector”, as used herein, refers to a recombinant nucleic acid for transferring exogenous nucleotide sequences (for example a nucleotide sequences encoding the cytochrome P450 monooxygenase enzyme as described herewith) into host or host cells (e.g. yeast or plant cells) and directing expression of the exogenous nucleic acid sequences in the host or host cells. “Expression cassette” refers to a nucleic acid comprising a nucleotide sequence of interest under the control of, and operably (or operatively) linked to, an appropriate promoter or other regulatory elements for transcription of the nucleic acid of interest in a host cell. As one of skill in the art would appreciate, the expression cassette may comprise a termination (terminator) sequence that is any sequence that is active the host cell (e.g. yeast or plant host).

Vectors suitable for different hosts are well known within the art. Non-limiting examples of vectors include pCambia vectors, pEAQ, pJL-TRBO, pJL-TRBO-G, pJL-TRBO-PBC, pEAQ, pHREAQ (plants); baculovirus expression vector (insect); pESC, pESC-Leu2d vector (yeast); pOPINA-F, pQEs, pRSETs, pETs (bacteria) and they may be used known methods, and information provided by the manufacturer's instructions.

The vector or construct comprising a sequence encoding the cytochrome P450 monooxygenase may further comprise one or more expression enhancer or one or more regulatory region active in the host or host cell.

The vector or construct may be transfected by methods known in the art, including for example electroporation, microinjection, impalefection, hydrostatic pressure, continuous infusion, sonication, lipofection, and various other chemical, non-chemical, mechanical, or passive transfection approaches.

Transient expression methods may be used to express the vector or construct of the present disclosure (see Liu and Lomonossoff, 2002, Journal of Virological Methods, 105:343-348; which is incorporated herein by reference). Alternatively, a vacuum-based transient expression method, as described by Kapila et al., 1997, which is incorporated herein by reference) may be used. These methods may include, for example, but are not limited to, a method of Agroinoculation or Agroinfiltration, syringe infiltration, however, other transient methods may also be used as noted above. With Agro-inoculation, Agroinfiltration, or syringe infiltration, a mixture of Agrobacteria comprising the desired nucleic acid, for example the vector or construct of the present disclosure, enter the intercellular spaces of a tissue, for example the leaves, aerial portion of the plant (including stem, leaves and flower), other portion of the plant (stem, root, flower), or the whole plant. After crossing the epidermis the Agrobacteria infect and transfer t-DNA copies into the cells. The t-DNA is episomally transcribed and the mRNA translated, leading to the production of the cytochrome P450 monooxygenase in infected cells. However, the passage of t-DNA inside the nucleus is transient.

### Host

The cytochrome P450 monooxygenase as described herewith may be produced or expressed within a host or host cell. The host or host cell may be a transgenic host or host cell. The transgenic host or host cell may comprise a vector or nucleic acid comprising a nucleotide sequence that encodes the cytochrome P450 monooxygenase as described herewith.

Since many hosts display a bias for use of particular codons to code for insertion of a particular amino acid in a growing peptide chain, the nucleotide sequence that encodes the cytochrome P450 monooxygenase may have been codon optimized for example the sequences have been optimized for plant codon usage or yeast codon usage.

“Codon optimization” is defined as modifying a nucleic acid sequence for enhanced expression in a host or host cell of interest by replacing at least one, more than one, or a significant number, of codons of the native sequence with codons that may be more frequently or most frequently used in the genes of another organism or species. Various species exhibit particular bias for certain codons of a particular amino acid.

There are different codon-optimization techniques known in the art for improving, the translational kinetics of translationally inefficient protein coding regions. These techniques mainly rely on identifying the codon usage for a certain host organism. If a certain gene or sequence should be expressed in this organism, the coding sequence of such genes and sequences will then be modified such that one will replace codons of the sequence of interest by more frequently used codons of the host organism.

The codon optimized polynucleotide sequences of the present disclosure may then be expressed in the host for example plants or yeast as described below.

The one or more than one modified genetic constructs of the present description may be expressed in any suitable host or host cell that is transformed by the nucleic acids, or nucleotide sequence, or constructs, or vectors of the present disclosure. The host or host cell may be from any source including plants, fungi, bacteria, insect, microalgae (Euglena, Chlamydomonas, etc) and animals. Therefore the host or host cell may be selected from a plant or plant cell, a fungi or a fungi cell, a bacteria or bacteria cell, an insect or an insect cell, and animal or an animal cell. In a preferred embodiment the host or host cell is a yeast cell, plant, portion of a plant or plant cell.

The host or host cell may be in a cell culture, for example in culture suspension or in a bioreactor wherein the MIA substrate for oxidation or hydroxylation is provided as a substrate or feed stock, or provided in the cell medium or cell culture itself. The host cells within the cell culture may accordingly comprise transformed, transgenic, or genetically modified cells suited for growth in the cell culture medium or conditions. For example, the host cells of the cell culture may be bacteria, yeast, plant or fungi cells transformed to express the vector or construct of the present disclosure. For example, the host cell of the cell culture may be transformed or transgenic Saccharomyces cerevisiae, Escherichia coli or a plant suspension culture.

The host or host cell may be cultured in batch, fed-batch, and continuous fermentation conditions. Culturing of the host or host cell may be appropriately scaled to various bioreactor conditions suitable for the production or activity of the cytochrome P450 monooxygenase. The cytochrome P450 monooxygenase may be retained within the host or host cell or may be secreted into the culture or bioreactor medium. The medium may or may not contain a suitable substrate for the cytochrome P450 monooxygenase. The cytochrome P450 monooxygenase may be recovered or purified from the host or host cell or the culture or bioreactor medium. Enzymatic products of the cytochrome P450 monooxygenase may be recovered or purified from the host or host cell or the culture or bioreactor medium using conventional techniques known within the art. For example, the cytochrome P450 monooxygenase or its enzymatic products may be recovered by filtration, centrifugation, ultrafiltration, dehydration, or a combination of steps thereof. For example the cytochrome P450 monooxygenase may be recovered from microsomal fractions prepared from cultures of the host or host cell. Recovery of the cytochrome P450 monooxygenase from the host or host cell may, for example, follow a combination of steps comprising centrifugation and/or lysing of the host or host cell, high-speed centrifugation to obtain a fraction containing microsomes, resuspension of microsomes.

The term “plant”, “portion of a plant”, “plant portion’, “plant matter”, “plant biomass”, “plant material”, plant extract”, or “plant leaves”, as used herein, may comprise an entire plant, tissue, cells, or any fraction thereof, intracellular plant components, extracellular plant components, liquid or solid extracts of plants, or a combination thereof, that are capable of providing the transcriptional, translational, and post-translational machinery for expression of one or more than one nucleic acids described herein, and/or from which an expressed protein and/or hydroxylated MIA product may be extracted and purified.

Plants may include, but are not limited to, herbaceous plants. The herbaceous plants may be annuals, biennials or perennials plants. Plants may include Camptotheca spp., for example Camptotheca acuminata, Ophiorrhiza spp., for example Ophiorrhiza pumila, Notapodytes spp., for example Nothapodytes nimmoniana, and members of the Nothapodytes, Ophiorrhiza, Chonemorpha, Apodytes, Merillodendron, Dysoxylum , Tabernaemontana, Codiocarpus, Pyrenacantha, Mostuea, or Iodes genera. Plants may further include, but are not limited to agricultural crops including for example canola, Brassica spp., maize, Nicotiana spp., (tobacco) for example, Nicotiana benthamiana, Nicotiana rustica, Nicotiana, tabacum, Nicotiana alata, Arabidopsis thaliana, alfalfa, potato, sweet potato (Ipomoea batatus), ginseng, pea, oat, rice, soybean, wheat, barley, sunflower, cotton, corn, rye (Secale cereale), sorghum (Sorghum bicolor, Sorghum vulgare), safflower (Carthamus tinctorius).

Furthermore, the host or host cell may be a yeast. Saccharomyces cerevisiae is commonly used for heterologous and homologous recombinant enzyme expression and biopharmaceutical synthesis and protein production. Therefore the yeast may be Saccharomyces cerevisiae or a non-conventional yeast species including but not limited to Hansenula polymorpha, Pichia pastoris, Komagataella phaffii, Yarrowia hpolytica, Schizosaccharomyces pombe, and Kluyveromyces lactis or any other suitable yeast host or host cell for expression or synthesis of the cytochrome P450 monooxygenase, the camptothecin hydroxylase, or homologous enzymes. Further, the yeast host or host cell may be a genetically modified, recombinant, or synthetic variant, for example a genetically modified, recombinant, or synthetic variant of Saccharomyces cerevisiae, Hansenula polymorpha, Pichia pastoris, Komagataella phaffii, Yarrowia hpolytica, Schizosaccharomyces pombe, and Kluyveromyces lactis or any other suitable yeast host or host cell for expression or synthesis of the cytochrome P450 monooxygenase, the camptothecin hydroxylase, or other homologous enzymes. For example the yeast may be protease-deficient yeast strain, such as YPL 154C:Pep4KO, or a yeast strain with improved penetration for and resistance to topoisomerase I inhibitors, such the Δerg6 Δtop1 yeast double mutant strain SMY75-1.4A43.

The yeast host or host cell may be modified by introduction of integration one or more plasmids or vectors, including but not limited to (YIp), episomal plasmids (YEp), and centromeric plasmids (YCp). The yeast host or host cell may be manipulated or modified, for example by CRISPR-Cas9, zinc finger nucleases (ZFNs), transcription activator-like effector nucleases (TALENs), and other gene editing techniques known in the art. The yeast host or host cell may be modified by one or more or a combination of such methods in order to express the cytochrome P450 monooxygenase or improve expression, yield, stability, or purity thereof, or other commercially beneficial parameters for production of the cytochrome P450 monooxygenase and its substrates or products. The plasmid or vector may encode one or more secretion factors. The plasmid or vector may encode one or more chaperone proteins or helper proteins. The yeast host or host cell may also be modified to improve resistance or the host or host cell to products of the cytochrome P450 monooxygenase.

For example, the plasmid or vector may be the yeast episomal plasmidpESC-Leu2d. The plasmid or vector may be designed such that the cytochrome P450 monooxygenase is inserted in the plasmid or vector in manner for expression. Furthermore, The plasmid or vector may be designed to comprise one or more promoters for improved or functional expression of the cytochrome P450 monooxygenase. For example, the plasmid or vector may comprise ADH1, GAPDH, PGK1, TPI, ENO, PYK1, TEF, GAL1-10, CUP1, ADH2, PGK, LAC4, ADH4, TEF, RPS7, XPR2/hp4d, PDX2, POT1, ICL1, GAP, TEF, PGK, YPT1, AOX1, FLD1, PEX8, or other promoters, enhancers, or promoter elements known in the art. The promoter may be constitutive or inducible.

Yeast may express and post-translationally modify recombinant proteins and enzymes. Accordingly, the yeast host or host cell may be modified to alter expression levels or post-translational modifications to the cytochrome P450 monooxygenase, the camptothecin hydroxylase, or a homologous enzyme expressed in the host or host cell. The post-translational modifications may include, for example, acetylation, amidation, hydroxylation, methylation, N-linked glycosylation, 0-linked glycosylation, phosphorylation, pyrrolidone carboxylic acid, sulfation, and ubiquitylation of the cytochrome P450 monooxygenase, the camptothecin hydroxylase, or the homologous enzyme for improved availability, purity, enzymatic function, stability, bioactivity, or other commercially beneficial parameters.

The host or host cell may be modified to increase production of a MIA substrate. For example the host or host cell may be modified to decrease production of a natural occurring hydroxylated MIA to increase the production of the (non-hydroxylated) MIA. Alternatively, the host or host cell may be modified to increase production of a MIA product or hydroxylated MIA. The modification may comprise any modification known within the art. For example the modification may be accomplished by silencing/knockout techniques that are known within the art for example by RNAi, VIGS, TALEN or CRISPR.

The term “increased production” (also referred to as “overproduction”) may describe an increase in the production of hydroxylated MIA in a host or host cell expressing or overexpressing a recombinant cytochrome P450 monooxygenase as described herewith. For example, naturally occurring plant such as Camptotheca accuminata, Ophiorrhiza pumila or Nothapodytes nimmoniana may be biologically engineered to express or overexpress a cytochrome P450 monooxygenase as described herewith so that production of hydroxylated MIA in the engineered plant may be increased over the production of hydroxylated MIA that is naturally occurring in the plant.

The transgenic host or host cell expressing the cytochrome P450 monooxygenase may be used in an in vivo method or process (also referred to as ‘in vivo enzymatic conversion’) for producing a MIA product as further described below.

In another aspect of the disclosure, the cytochrome P450 monooxygenase may be purified or extracted from the transgenic host. For example, cytochrome P450 monooxygenase may be extracted as microsomal proteins in microsomal fractions. The purified cytochrome P450 monooxygenase may be used for an in vitro method or process (also referred to as ‘in vitro enzymatic conversion’) for producing a MIA product as further described below.

### Substrate

The cytochrome P450 monooxygenase enzymes as described herewith is capable of oxidizing a monoterpenoid indole alkaloid (MIA) substrate to produce a MIA product. The MIA product may be a hydroxylated MIA (HMIA) or a dihydroxylated MIA (DMIA). As described above the MIA comprises either a quinoline moiety (also referred to as “quinoline MIA”) or a indole moiety (also referred to as “indole MIA”).

As described above, the cytochrome P450 monooxygenase enzyme may catalyze the oxidation of carbon C5, C6, or C7 of the quinoline moiety in the MIA or the cytochrome P450 monooxygenase enzyme may catalyze the oxidation of C4, C5 or C6 of the indole moiety in the MIA to produce hydroxylated MIA.

### Camptothecinoid Substrate

For example the quinoline moiety comprising MIA might be a camptothecinoid substrate. The cytochrome P450 monooxygenase enzymes as described herewith catalyze the oxidation of C9, C10 or C11 of the ‘camptothecinoid substrate’ to produce a ‘camptothecinoid product’ (e.g. a hydroxylated camptothecinoid). In some instances the camptothecinoid substrate might already be hydroxylated at position C9, C10 or C11. Therefore the camptothecinoid substrate may also be a hydroxylated camptothecinoid to produce a dihydroxylated camptothecinoid.

The term “camptothecinoid” as used herein, may refer to camptothecin and camptothecin analogues and derivatives. The camptothecin analog may be a structural or a functional analog. Camptothecinoid is a pentacyclic monoterpenoid indole alkaloid (MIA) with a quinoline moiety. The camptothecinoid may have a planar pentacyclic ring structure, that includes a pyrrolo[3,4-β]-quinoline moiety (rings A, B and C), conjugated pyridone moiety (ring D) and one chiral center at position 20 within the alpha-hydroxy lactone ring with (S) configuration (the E-ring). Without wishing to be bound by theory, it is believed that the planar structure is one of the most important factors for the ability of camptothecinoids to inhibit topoisomerase.

Camptothecinoid may comprise the general scaffold or ring system of Formula A:

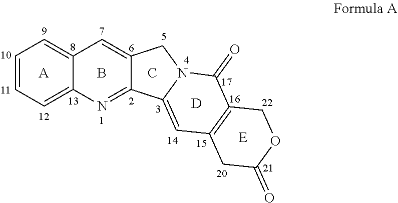

The general scaffold or ring system of Formula A may also be referred to as camptothecin scaffold or a CPT scaffold.

The camptothecinoid may comprise one or more substitutions to the CPT scaffold and/or optional moieties that are covalently attached to the CPT scaffold. Examples of substitutions to the CPT scaffold that may also be present in the camptothecinoid include nitrogen, oxygen, and the like. Examples of optional moieties that may be covalently attached to the CPT scaffold include but are not limited to methyl, ethyl, carboxylic acid, amine, acid amine, chloride, acid chloride, alcohol, aldehyde, ketone, ester, ether, any halide (including F, Cl, Br, and I), nitrile, nyanide, nitro, sufide, sulphonic acid, and thiol groups. Other moieties that may be covalently attached to the CPT scaffold include but are not limited to any C₁₋₂₀ linear or cyclic alkyl, cyclic or polycyclic compounds derived from cyclopropane, cyclobutane, cyclopentane, cyclohexane, cycloheptane, cyclooctane, cyclenonane, cyclodecane, including benezene and any aromatic groups, as well as any substituted or functionalized derivatives thereof. The optional moieties may include naturally occurring moieties or any ionized, substituted, or synthetic moieties or analogs thereof.

The camptothecinoid may furher comprise negatively-charged bulky groups at positions 9, 10, and/or 11, which may increase the inhibitory activity of camptothecinoid against topoisomerase I (Lu et al. Acta Pharmacol Sin 2007 February; 28(2): 307-314, which is herewith incorporated by reference). The camptothecinoid may also comprise substitutents carrying large positively-charged group at position C-7, which may also enhance the inhibitory activity of the camptothecinoid against topoisomerase I (Verma & Hansch, Chem. Rev. 2009, 109, 1, 213-235, which is herewith incorporated by reference).

The camptothecinoid may comprise one or more optional moieties as defined above covalently attached at position C-7, C-9, C-10, and/or C-11. The camptothecinoid may comprise one or more substitutions as defined above at position C-7, C-9, C-10, and/or C-11. For the purpose of illustration only, and not limiting the scope of the present invention, a few examples of the camptothecinoid are shown in Formula B:

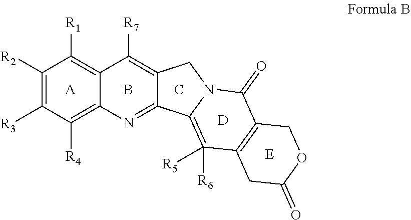

Wherein, for example, R₁, R₂, R₃, R₄, R₅, and R₆ may be hydrogen, hydroxy, halogen, amine, C₁₋₂₀ linear or cyclic alkyl (which optionally may be further substituted), —OR₇, —OC(═O)R₈, or a glucopyranosyl; wherein R₇ may be a linear alkyl or a protecting group, such as e.g. acetate (Ac); and wherein R₈ may be a C₁₋₂₀ linear alkyl.

The camptothecinoid substrate of the cytochrome P450 monooxygenase enzyme may be for example camptothecinoid, 10-hydroxycamptothecinoid, 11-hydroxycamptothecinoid, 7-ethyl camptothecinoid, 9-amino-camptothecinoid, 9-nitro-camptothecinoid or 9-hydroxycamptothecinoid. In an embodiment the camptothecinoid substrate may be camptothecin, 9-hydroxycamptothecin, 10-hydroxycamptothecin, 11-hydroxycamptothecin, 7-ethylcamptothecin, 9-amino-camptothecin or 9-nitro-camptothecin. For example, in one embodiment the substrate may be camptothecin, 10-hydroxycamptothecin, 7-ethylcamptothecin or 9-amino-camptothecin.

The camptothecinoid may be camptothecin and may comprises the general ring system of Formula A1:

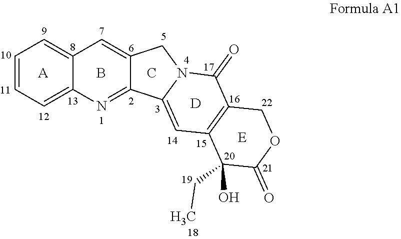

10-hydroxycamptothecinoid

The camptothecinoid may be a “10-hydroxycamptothecinoid”.

10-hydroxycamptothecinoid refers to a compound which comprises the general ring system of Formula B1:

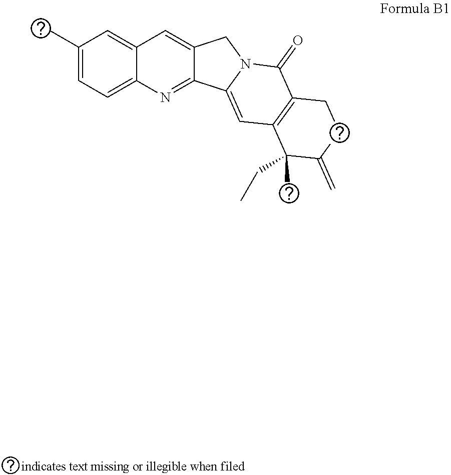 [SMILES: C=C1CCc2c(cc3n(c2=O)Cc2cc4cc(C)ccc4nc2-3)[C@@]1(C)CC]

A non-limiting example of a 10-hydroxycamptothecinoid is 10-hydroxycamptothecin.

7-ethylcamptothecinoid

7-ethylcamptothecinoid refers to a compound which comprises the general ring system of

Formula B2:

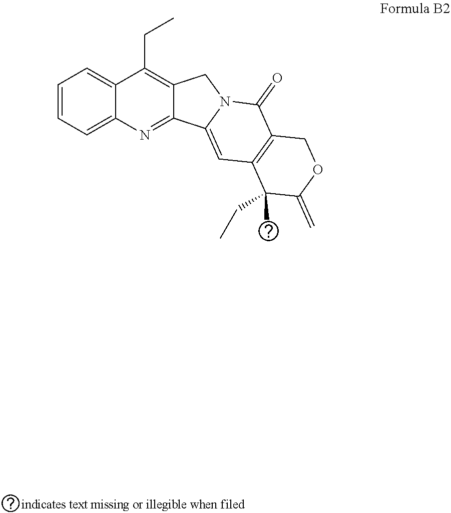 [SMILES: C=C1OCc2c(cc3n(c2=O)Cc2c-3nc3ccccc3c2CC)[C@@]1(C)CC]

A non-limiting example of a 7-ethylcamptothecinoid is 7-ethylcamptothecin.

9-amino-camptothecinoid

The camptothecinoid may be a “9-amino-camptothecinoid”

9-amino-camptothecinoid refers to a compound which comprises the general ring system of Formula B3:

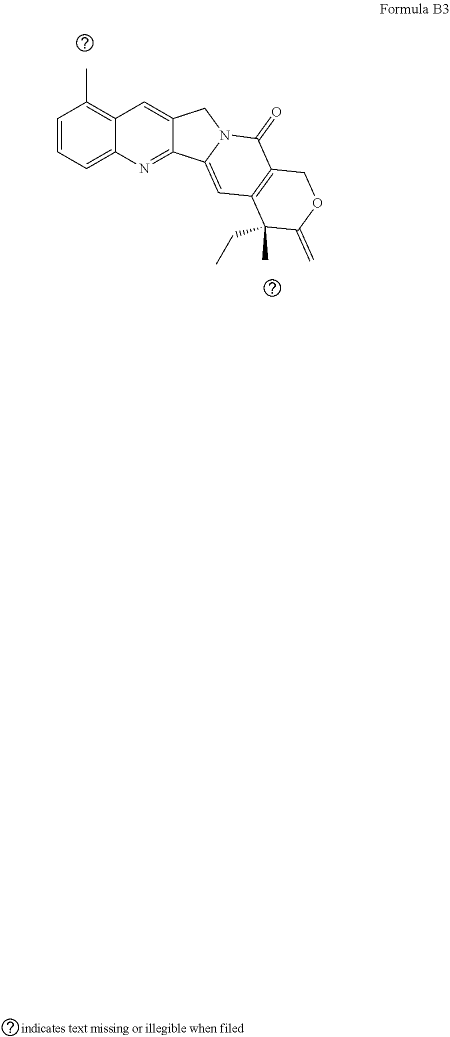 [SMILES: C=C1OCc2c(cc3n(c2=O)Cc2cc4c(C)cccc4nc2-3)[C@@]1(C)CC]

A non-limiting example of a 9-amino-camptothecinoid is 9-amino-camptothecin.

9-hydroxycamptothecinoid

The camptothecinoid may be a “9-hydroxycamptothecinoid”.

9-hydroxycamptothecinoid refers to a compound which comprises the general ring system Of Formula B4:

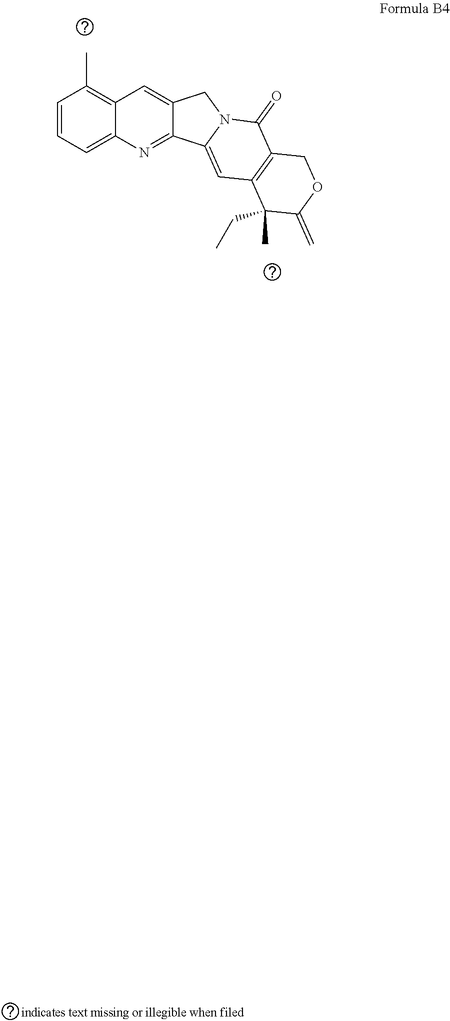 [SMILES: C=C1OCc2c(cc3n(c2=O)Cc2cc4c(C)cccc4nc2-3)[C@@]1(C)CC]

A non-limiting example of a 9-hydroxycamptothecinoid is 9-hydroxycamptothecin.

### Evodiaminoid

The indole moiety comprising compound (MIA) or MIA substrate might be an evodiaminoid.

The cytochrome P450 monooxygenase enzymes as described herewith may catalyze the oxidation of C9, C10 or C11 of the ‘evodiaminoid substrate’ to produce a ‘evodiaminoid product’ (e.g. a hydroxylated evodiaminoid). In some instances the evodiaminoid substrate might already be hydroxylated at one or more than one position at C9, C10 or C11. Therefore the evodiaminoid substrate may also be a hydroxylated evodiaminoid, which may yield to for example a dihydroxylated evodiaminoid product.

The evodiaminoid substrate of the cytochrome P450 monooxygenase enzyme may be for example evodiaminoid, 9-hydroxy evodiaminoid, 10-hydroxyevodiaminoid, or 11-hydroxyevodiaminoid. In on embodiment the evodiaminoid substrate is an evodiaminoid, such for example a evodiamine.

The term “evodiaminoid ” as used herein, may refer to evodiamine and evodiamine analogues and derivatives. The evodiamine analog may be a structural or a functional analog. Evodiaminoid is a pentacyclic monoterpenoid indole alkaloid (MIA) with an indole moiety. The evodiaminoid may have a pentacyclic ring structure, that includes an indole moiety (rings A and B).

Evodiaminoid comprises the general scaffold or ring system of Formula C:

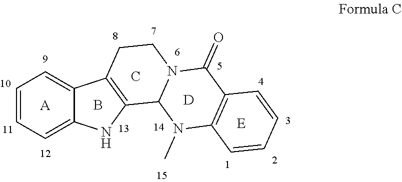

The general scaffold or ring system of Formula C may also be referred to as evodiamine scaffold.

The evodiaminoid may comprise one or more substitutions to the evodiamine scaffold and/or optional moieties that are covalently attached to the evodiamine scaffold. Examples of substitutions to the evodiamine scaffold that may also be present in the evodiaminoid include nitrogen, oxygen, and the like. Examples of optional moieties that may be covalently attached to the evodiamine scaffold include but are not limited to methyl, ethyl, carboxylic acid, amine, acid amine, chloride, acid chloride, alcohol, aldehyde, ketone, ester, ether, any halide (including F, Cl, Br, and I), nitrile, nyanide, nitro, sufide, sulphonic acid, and thiol groups. Other moieties that may be covalently attached to the evodiamine scaffold include but are not limited to any C₁₋₂₀ linear or cyclic alkyl, cyclic or polycyclic compounds derived from cyclopropane, cyclobutane, cyclopentane, cyclohexane, cycloheptane, cyclooctane, cyclenonane, cyclodecane, including benezene and any aromatic groups, as well as any substituted or functionalized derivatives thereof. Further moieties that might be attached include trifluoromethyl, trifluoromethoxy, methoxyl group, oxyethyl group, propoxy-, isopropoxy or butoxy; Lower hydroxy alkyl, low-grade alkyl amino, low-grade halogenated alkyl are amino, low-grade cycloalkyl is amino, alkynyl of low-grade chain is amino, amide group, low-grade cycloalkyl amide group, rudimentary amido alkyl; With Boc and the amino acid sloughing Boc; hydrogen, halogen, low-grade halogenated alkyl, low alkyl group, hydroxyl, Lower hydroxy alkyl, lower alkoxy, amino, low-grade alkyl amino, low-grade halogenated alkyl are amino, low-grade cycloalkyl is amino, alkynyl of low-grade chain is amino, amide group, low-grade cycloalkyl amide group, or rudimentary amido alkyl (see for example CN105418610, which is incorporated by reference) The optional moieties may include naturally occurring moieties or any ionized, substituted, or synthetic moieties or analogs thereof.

### Ellipticinoid

The indole moiety comprising compound (MIA) or substrate might further be derived from an ellipticinoid.

The cytochrome P450 monooxygenase enzymes as described herewith may catalyze the oxidation of C7, C8, C9, C10, C12, or C13 of the ‘ellipticinoid substrate’ to produce a ‘ellipticinoid product’ (e.g. a hydroxylated ellipticinoid). In some instances the ellipticinoid substrate might already be hydroxylated at position C7, C8, C9, C10, C12, or C,13 to produce a dihydroxylated ellipticinoid product. Therefore the ellipticinoid substrate may also be a hydroxylated ellipticinoid. In one embodiment the cytochrome P450 monooxygenase enzymes may catalyze the oxidation of C8, C9, and/or C10 of the ‘ellipticinoid substrate’ to produce a ‘ellipticinoid product’ (e.g. a hydroxylated ellipticinoid).

The ellipticinoid substrate of the cytochrome P450 monooxygenase enzyme may be for example ellipticinoid, 7-hydroxy ellipticinoid, 8-hydroxy ellipticinoid, 9-hydroxy ellipticinoid, 10-hydroxy ellipticinoid, 12-hydroxy ellipticiboid, 13-hydroxy ellipticinoid. In on embodiment the ellipticinoid substrate is an ellipticinoid, such for example a ellipticine.

The term “ellipticinoid” as used herein, may refer to ellipticine and ellipticine analogues and derivatives. The ellipticine analog may be a structural or a functional analog. Ellipticinoid is a pentacyclic monoterpenoid indole alkaloid (MIA) with an indole moiety. The ellipticinoid may have a planar pentacyclic ring structure, that includes an indole moiety (rings A and B).

Ellipticinoid comprises the general scaffold or ring system of Formula D:

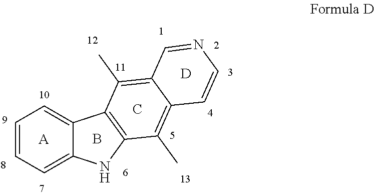

The general scaffold or ring system of Formula D may also be referred to as ellipticinoid scaffold.

The ellipticinoid may comprise one or more substitutions to the ellipticinoid scaffold and/or optional moieties that are covalently attached to the ellipticinoid scaffold. Examples of substitutions to the ellipticinoid scaffold that may also be present in the ellipticinoid include nitrogen, oxygen, and the like. Examples of optional moieties that may be covalently attached to the ellipticinoid scaffold include but are not limited to methyl, ethyl, carboxylic acid, amine, acid amine, chloride, acid chloride, alcohol, aldehyde, ketone, ester, ether, any halide (including F, Cl, Br, and I), nitrile, nyanide, nitro, sufide, sulphonic acid, and thiol groups. Other moieties that may be covalently attached to the ellipticinoid scaffold include but are not limited to any C₁₋₂₀ linear or cyclic alkyl, cyclic or polycyclic compounds derived from cyclopropane, cyclobutane, cyclopentane, cyclohexane, cycloheptane, cyclooctane, cyclenonane, cyclodecane, including benezene and any aromatic groups, as well as any substituted or functionalized derivatives thereof. The optional moieties may include naturally occurring moieties or any ionized, substituted, or synthetic moieties or analogs thereof.

### Method of Producing HMIA

The present description further relates to a method or process for producing a MIA product (for example a hydroxylated MIA or dihydroxylated MIA). The method or process comprises contacting the MIA substrate (as described above) with the cytochrome P450 monooxygenase as described herewith under conditions suitable for oxidation or hydroxylation of the MIA substrate, thereby forming a MIA product.

The MIA substrate may be contacted with the cytochrome P450 monooxygenase under conditions suitable for oxidation or hydroxylation of the MIA by the cytochrome P450 monooxygenase. The contacting may occur in vitro or in vivo.

For example the contacting may occur in a container vial, vessel, bioreactor, or the like in which conditions suitable for oxidation or hydroxylation are induced or observed. The contacting may occur in a medium with or without cells. Alternatively, the contacting may occur within any suitable host or host cell comprising a vector or construct for expressing the cytochrome P450 monooxygenase as described above.

The method or process of producing a MIA product (such as a hydroxylated MIA or dihydroxylated MIA) in a host or host cell may comprise the introduction of a nucleic acid comprising a sequence encoding a cytochrome P450 monooxygenase as described herewith, into a host or host cell, and incubating the host or host cell under conditions that permit the expression of the nucleic acid, thereby producing the cytochrome P450 monooxygenase.

In a further step, the host or host cell expressing the cytochrome P450 monooxygenase is contacted with the MIA substrate to produce the MIA product (‘in vivo enzymatic conversion’). The contacting may for example comprise culturing the host or host cell in the presence of the MIA substrate or infiltrating the substrate into the host or host cell.

Accordingly, it is also provided a method or process for producing a MIA product as described herewith, wherein the steps comprise i. providing a host or host cell, for example a transgenic host cell comprising a nucleic acid comprising a sequence encoding a cytochrome P450 monooxygenase as described herewith, ii. culturing or incubating the host or host cell under condition suitable for the expression of cytochrome P450 monooxygenases enzyme and iii. contacting the host or host cell with a MIA substrate to produce a MIA product. The MIA product may further be recovered from the host or host cell. The MIA product may be further reacted as described below. The cytochrome P450 monooxygenase may for example be CPT9H, CPT10H or CPT11H.

Alternatively, the host or host cell expressing the cytochrome P450 monooxygenase may be processed to produce an extract that comprises the cytochrome P450 monooxygenase. The extract may be used to contact the MIA substrate (‘in vitro enzymatic conversion with extract’).

Furthermore, the cytochrome P450 monooxygenase may be extracted, purified or extracted and purified from the host or host cell extract and the MIA substrate may be contacted with the purified cytochrome P450 monooxygenase (‘in vitro enzymatic conversion with purified enzyme’).

The following non-limiting examples of methods or processes are provided:

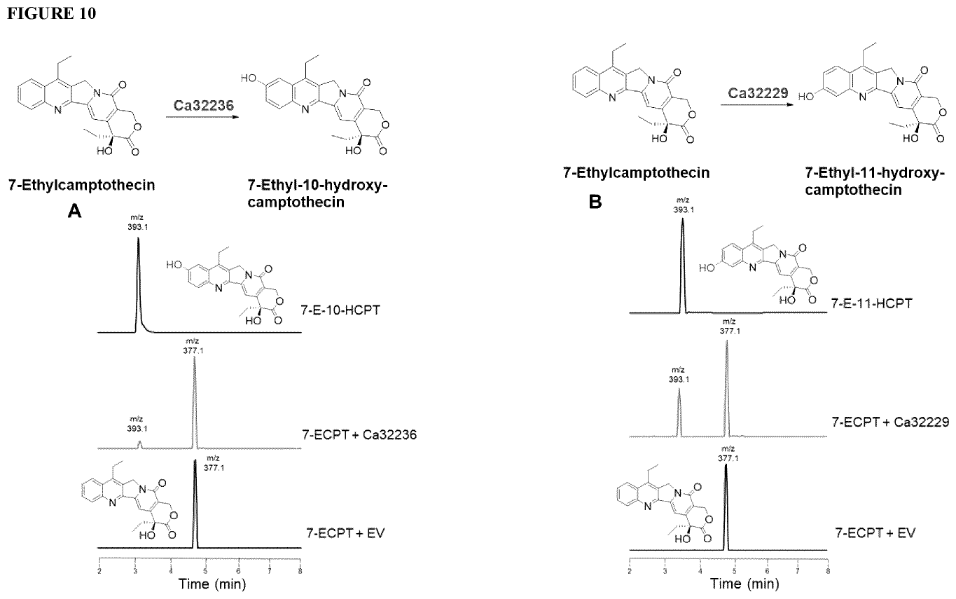

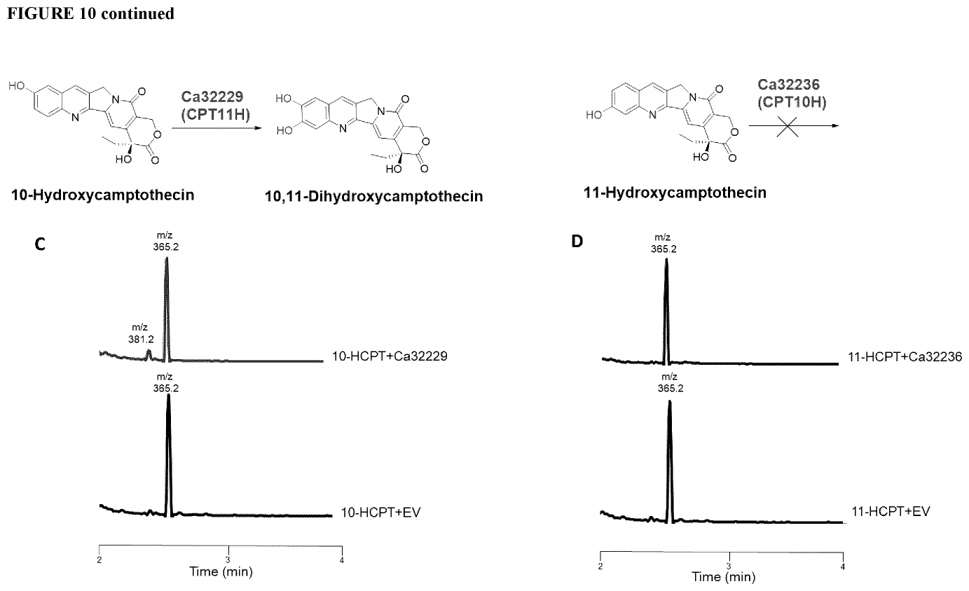

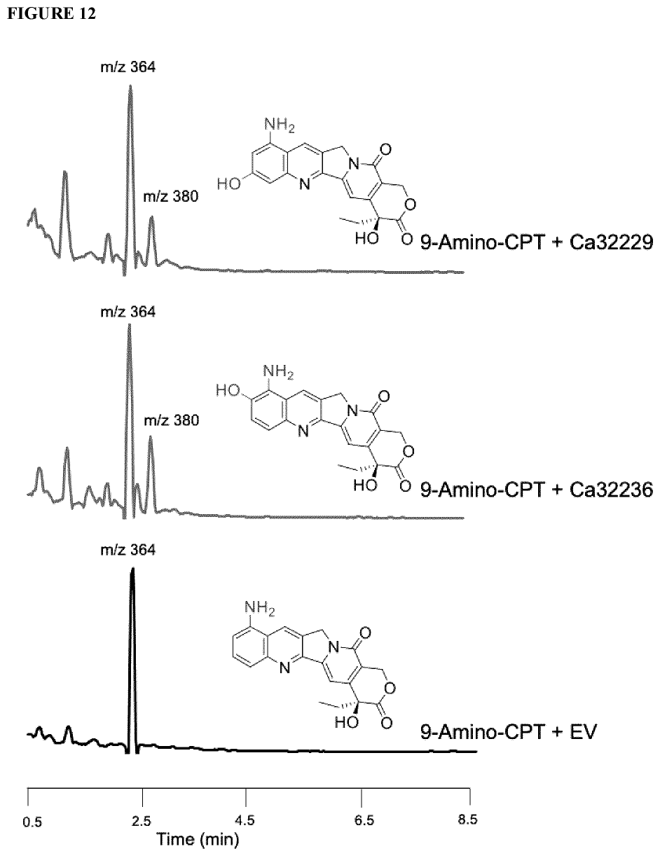

As shown in FIGS. 1 and 2A and Example 4,10-hydroxycamptothecin may be produced from camptothecin by contacting camptothecin with CPT10H enzyme (Ca32236). In another non-limiting example, as shown in FIG. 10A and Example 4,7-ethyl-10-hydroxycamptothecin may be produced from 7-ethylcamptothecin by contacting 7-ethylcamptothecin with CPT10H enzyme (Ca32236). Furthermore, FIG. 12 shows the production of 9-amino-10-hydroxycamptothecin by contacting 9-amino-camptothecin with CPT10H enzyme (Ca32236).

Accordingly, it is also provided a method or process for producing a 10-hydroxycamptothecinoid, the method comprising contacting a camptothecinoid with a cytochrome P450 monooxygenase as described herewith (for example CPT10H) under conditions suitable for oxidation or hydroxylation of the camptothecinoid to produce a 10-hydroxycamptothecinoid and optionally, isolating, purifying or recovering and/or further reacting the 10-hydroxycamptothecinoid.

It is also provided a method or process for producing a 7-ethyl-10-hydroxycamptothecinoid, the method comprising contacting a 7-ethylcamptothecinoid with a cytochrome P450 monooxygenase as described herewith (for example CPT10H) under conditions suitable for oxidation or hydroxylation of the 7-ethylcamptothecinoid to produce a 7-ethyl-10-hydroxycamptothecinoid and optionally, isolating, purifying or recovering and/or further reacting the 7-ethyl-10-hydroxycamptothecinoid.

Furthermore, it is also provided a method or process for producing a 9-amino-10-hydroxycamptothecinoid, the method comprising contacting a 9-amino-camptothecinoid with a cytochrome P450 monooxygenase as described herewith (for example CPT10H) under conditions suitable for oxidation or hydroxylation of the 9-amino-camptothecinoid to produce a 9-amino-10-hydroxycamptothecinoid and optionally, isolating, purifying or recovering and/or further reacting the 9-amino-10-hydroxycamptothecinoid.

As further shown in FIGS. 1 and 2B and Example 4, 11-hydroxycamptothecin may be produced from camptothecin by contacting camptothecin with CPT11H enzyme (Ca32229). In another non-limiting example, as shown in FIG. 10B and Example 4, 7-ethyl-11-hydroxycamptothecin may be produced from 7-ethylcamptothecin by contacting 7-ethylcamptothecin with CPT11H enzyme (Ca32229). Furthermore, as shown in FIG. 12, 9-amino-11-hydroxycamptothecin may be produced from 9-amino-camptothecin by contacting 9-amino-camptothecin with CPT11H enzyme (Ca32229).

Accordingly, it is further provided a method or process of producing a 11-hydroxycamptothecinoid, the method comprising contacting a camptothecinoid with at least one cytochrome P450 monooxygenase as describe herewith (for example CPT11H) under conditions suitable for oxidation or hydroxylation of the camptothecinoid to produce a 11-hydroxycamptothecinoid and optionally, isolating, purifying or recovering and/or further reacting the 11-hydroxycamptothecinoid.

As shown in FIG. 10C and Example 4, 10-hydroxycamptothecinoid may further be hydroxylated to 10,11-hydroxycamptothecinoid, by contacting 10-hydroxycamptothecino with CPT11H enzyme (Ca32229) to produce 10,11-hydroxycamptothecinoid.

It is therefore also provided a method or process, the method or process comprising contacting a first MIA substrate with a first cytochrome P450 monooxygenase under conditions suitable for oxidation or hydroxylation of the first MIA substrate, thereby forming a first MIA product. The first MIA product may be the substrate for a second enzymatic conversion. Therefor the ‘first MIA product’ may be a ‘second MIA substrate’. The first MIA product (or second MIA substrate) may be contacted with a second cytochrome P450 monooxygenase under conditions suitable for oxidation or hydroxylation of the first MIA product (second MIA substrate) thereby forming a second MIA product.

Alternatively, it is provided a method or process for producing a MIA product, wherein a MIA substrate is contacted by a first and second cytochrome P450 monooxygenase under conditions suitable for oxidation or hydroxylation of the MIA substrate, thereby forming a MIA product, wherein the MIA product is a dihydroxylated MIA product.

The first and second cytochrome P450 monooxygenase enzymes are different cytochrome P450 monooxygenase enzymes. For example the first cytochrome P450 monooxygenase may be CPT10H and the second cytochrome P450 monooxygenase may be CPT11H.

It is provided a method or process for producing a dihydroxylated MIA, wherein the steps comprise i. providing a first host or host cell comprising a first nucleic acid comprising a first sequence encoding a first cytochrome P450 monooxygenase as described herewith, ii. culturing the first host or host cell under condition suitable for the expression of the first cytochrome P450 monooxygenases enzyme, iii. contacting the first host or host cell with a MIA substrate to produce a first hydroxylated MIA product iv. providing a second host or host cell comprising a second nucleic acid comprising a second sequence encoding a second cytochrome P450 monooxygenase as described herewith, ii. culturing the second host or host cell under condition suitable for the expression of the second cytochrome P450 monooxygenases enzyme, iii. contacting the second host or host cell with the first hydroxylated MIA product to product a second hydroxylated MIA product, wherein the second hydroxylated MIA product is a dihydroxylated MIA.

Alternatively, the first host or host cell expressing the first cytochrome P450 monooxygenase may be processed to produce a first extract that comprises the first cytochrome P450 monooxygenase and the second host or host cell expressing the second cytochrome P450 monooxygenase may be processed to produce a second extract that comprises the second cytochrome P450 monooxygenase. The first and second extract may be used to contact the MIA substrate either consecutively or simultaneously to produce a dihydroxylated MIA product.

Furthermore, the first and second cytochrome P450 monooxygenase may be extracted, purified or extracted and purified from the first and second host or host cell to produce a purified first and second cytochrome P450 monooxygenase. The extracted or purified first and second cytochrome P450 monooxygenase may be used to contact the MIA substrate either consecutively or simultaneously to produce a dihydroxylated MIA product.

Furthermore, the method or process for producing a dihydroxylated MIA, may comprise
i. providing a host or host cell, for example a transgenic host cell comprising a first nucleic acid comprising a first sequence encoding a first cytochrome P450 monooxygenase as described herewith and a second nucleic acid comprising a second sequence encoding a second cytochrome P450 monooxygenase as described herewith and
ii. culturing the host or host cell under condition suitable for the expression of the first and second cytochrome P450 monooxygenases enzyme and
iii. contacting the host or host cell with a MIA substrate to produce a MIA product, wherein the MIA product is a dihydroxylated MIA product.

The MIA product may further be recovered from the host or host cell. The MIA may be further reacted as described below.

Alternatively, the host or host cell expressing the first and second cytochrome P450 monooxygenase may be processed to produce an extract that comprises the first and second cytochrome P450 monooxygenase. The extract comprising the first and second cytochrome P450 monooxygenase may be used to contact the MIA substrate either consecutively or simultaneously to produce a dihydroxylated MIA product.

Furthermore, the first and second cytochrome P450 monooxygenase may be extracted, purified or extracted and purified from the host or host cell to produce a purified first and second cytochrome P450 monooxygenase. The purified or extracted first and second cytochrome P450 monooxygenase may be used to contact the MIA substrate either consecutively or simultaneously to produce a dihydroxylated MIA product.

The first and second cytochrome P450 monooxygenase enzymes are different cytochrome P450 monooxygenase enzymes. For example the first cytochrome P450 monooxygenase may be CPT10H and the second cytochrome P450 monooxygenase may be CPT11H.

### Products

As described above, the present description relates to methods and processes to produce MIA products such for example hydroxylated MIA or dihydroxylated MIA products.

As used herein, a “hydroxylated MIA” is any MIA as described herewith wherein at least one hydroxyl group (OH) is attached to any one carbon of a MIA (See Table 1). A “dihydroxylated MIA” is any MIA as described herewith wherein two hydroxyl groups (OH) are attached to any carbon of a MIA.

For example the hydroxylated MIA may be a hydroxylated camptothecinoid, hydroxylated 7-ethyl camptothecinoid, hydroxylated 9-amino-camptothecinoid, hydroxylated 10-hydroxycamptothecinoid, hydroxylated evodiaminoid or hydroxylated ellipticinoid. The hydroxylated MIA may also be a hydroxylated hydroxycamptothecinoid (also referred to as dihydroxycamptothecinoid).

### Hydroxylated Camptothecinoid

For example, the hydroxylated camptothecinoid may be a 10-hydroxycamptothecinoid, which comprises the chemical structure of Formula B1, or functionalized or substituted variants thereof:

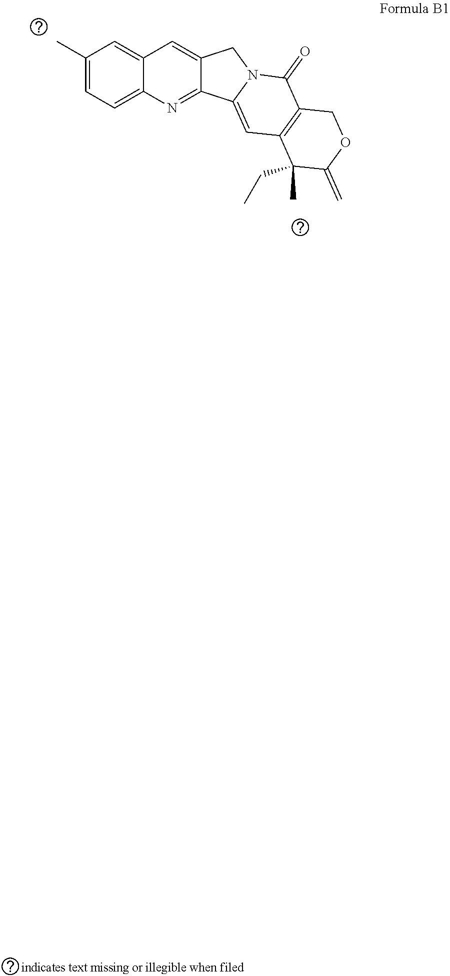 [SMILES: C=C1OCc2c(cc3n(c2=O)Cc2cc4cc(C)ccc4nc2-3)[C@@]1(C)CC]

Furthermore the hydroxylated camptothecinoid may be a 11-hydroxycamptothecinoid which comprises the chemical structure of Formula B5, or functionalized or substituted variants thereof:

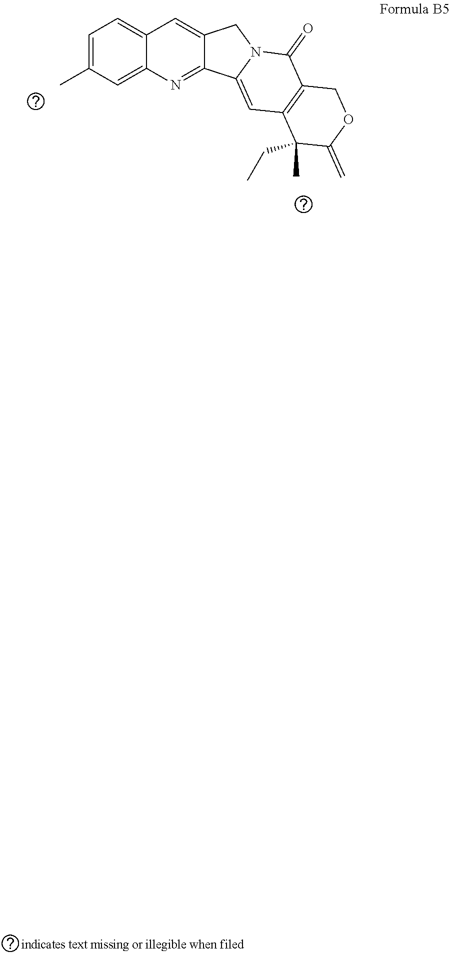 [SMILES: C=C1OCc2c(cc3n(c2=O)Cc2cc4ccc(C)cc4nc2-3)[C@@]1(C)CC]

The hydroxylated camptothecinoid may be a 9-hydroxycamptothecinoid which comprises the chemical structure of Formula B6, or functionalized or substituted variants thereof:

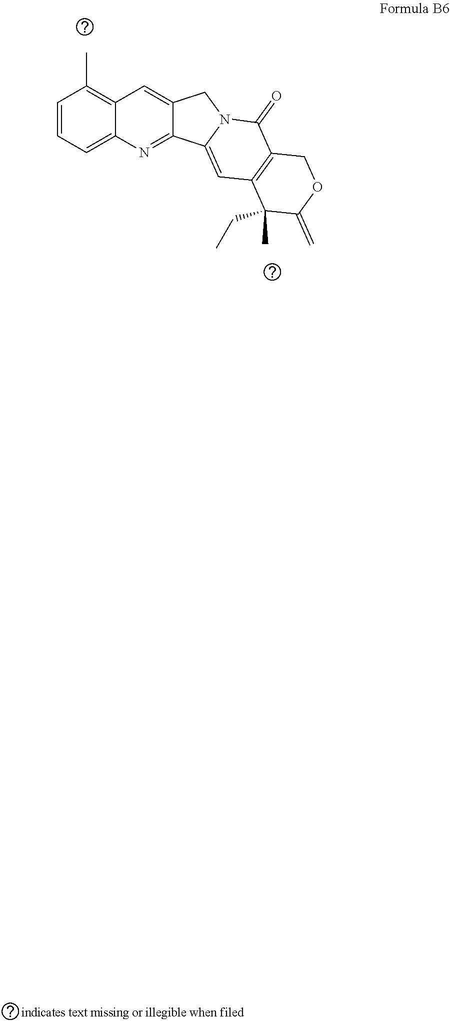 [SMILES: C=C1OCc2c(cc3n(c2=O)Cc2cc4c(C)cccc4nc2-3)[C@@]1(C)CC]

The hydroxylated camptothecinoid may be a 10,11-dihydroxycamptothecinoid which comprises the chemical structure of Formula B7, or functionalized or substituted variants thereof:

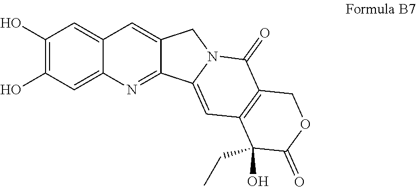 [SMILES: CC[C@@]1(O)C(=O)OCc2c1cc1n(c2=O)Cc2cc3cc(O)c(O)cc3nc2-1]

The hydroxylated camptothecinoid may be a 7-ethyl-9-hydroxycamptothecinoid which comprises the chemical structure of Formula B8, or functionalized or substituted variants thereof:

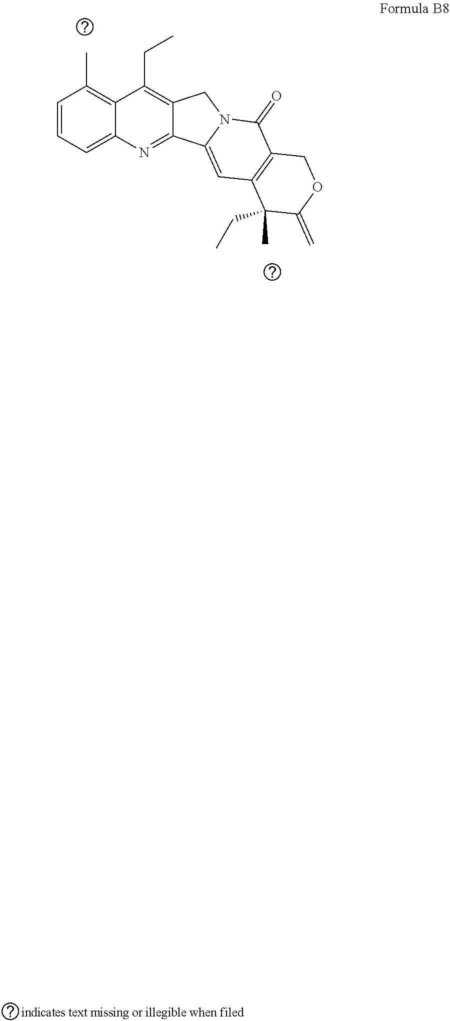 [SMILES: C=C1OCc2c(cc3n(c2=O)Cc2c-3nc3cccc(C)c3c2CC)[C@@]1(C)CC]

The hydroxylated camptothecinoid may be a 7-ethyl-10-hydroxycamptothecinoid which comprises the chemical structure of Formula B9, or functionalized or substituted variants thereof:

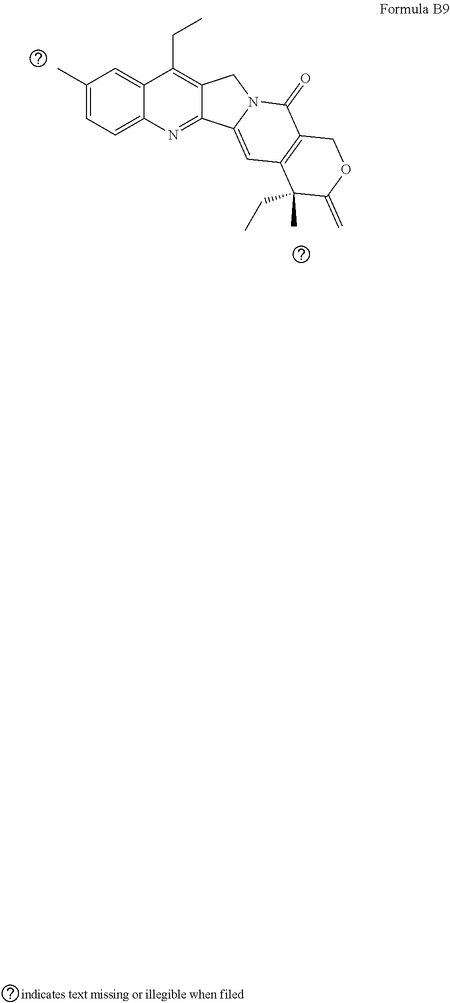 [SMILES: C=C1OCc2c(cc3n(c2=O)Cc2c-3nc3ccc(C)cc3c2CC)[C@@]1(C)CC]

The hydroxylated camptothecinoid may be a 7-ethyl-11-hydroxycamptothecinoid which comprises the chemical structure of Formula B10, or functionalized or substituted variants thereof:

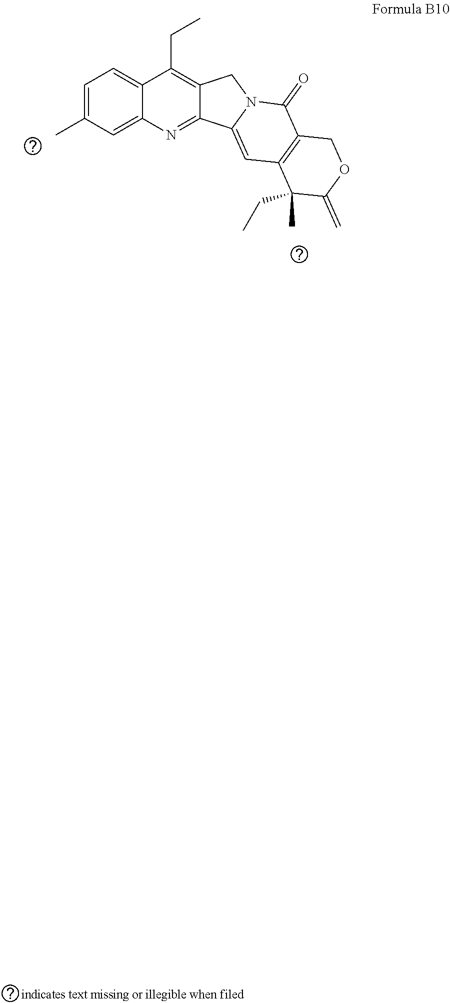 [SMILES: C=C1OCc2c(cc3n(c2=O)Cc2c-3nc3cc(C)ccc3c2CC)[C@@]1(C)CC]

The hydroxylated camptothecinoid may be a 7-ethyl-10,11-dihydroxycamptothecinoid which comprises the chemical structure of Formula B11, or functionalized or substituted variants thereof:

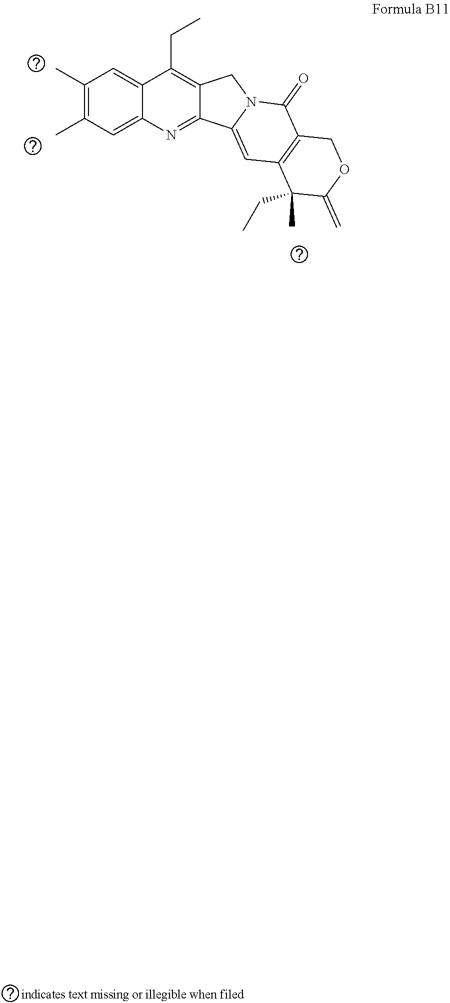 [SMILES: C=C1OCc2c(cc3n(c2=O)Cc2c-3nc3cc(C)c(C)cc3c2CC)[C@@]1(C)CC]

The hydroxylated camptothecinoid may be a 9-amino-10-hydroxycamptothecinoid which comprises the chemical structure of Formula B12, or functionalized or substituted variants thereof:

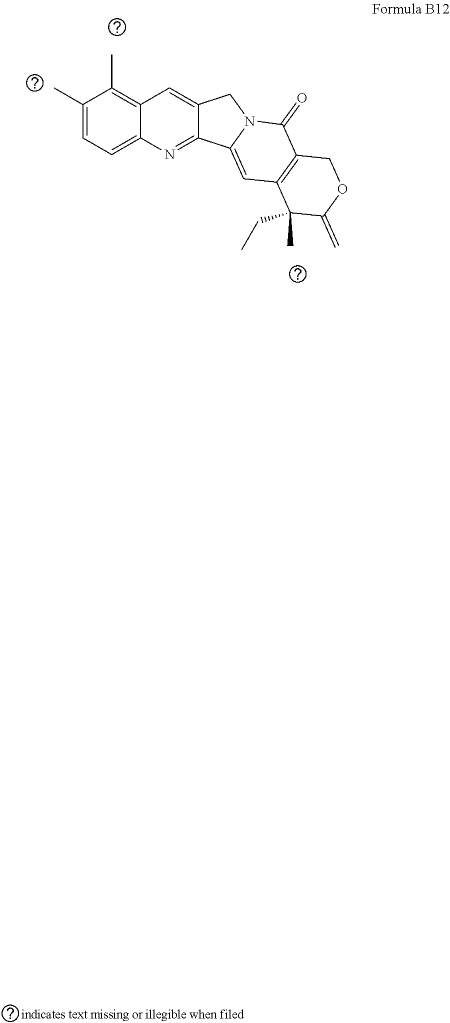 [SMILES: C=C1OCc2c(cc3n(c2=O)Cc2cc4c(C)c(C)ccc4nc2-3)[C@@]1(C)CC]

The hydroxylated camptothecinoid may be a 9-amino-11-hydroxycamptothecinoid which comprises the chemical structure of Formula B13, or functionalized or substituted variants thereof:

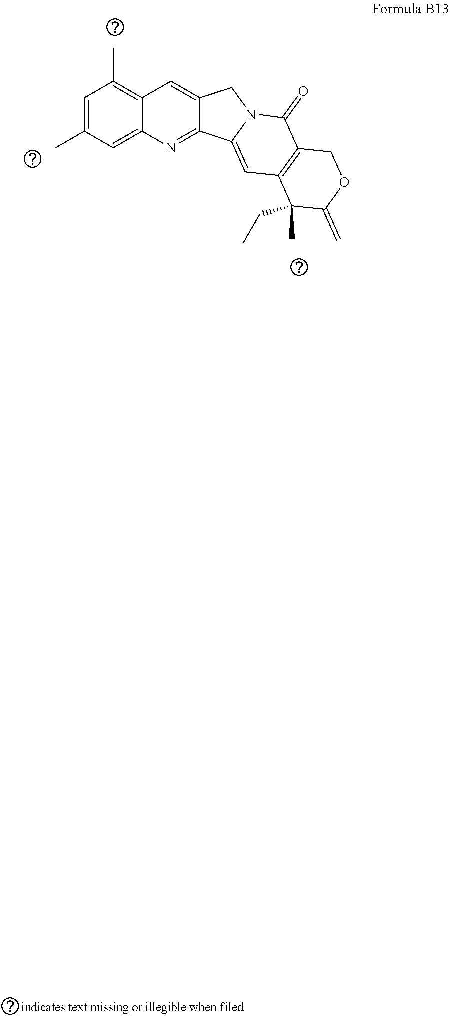 [SMILES: C=C1OCc2c(cc3n(c2=O)Cc2cc4c(C)cc(C)cc4nc2-3)[C@@]1(C)CC]

The hydroxylated camptothecinoid may be a 9-amino-10,11-dihydroxycamptothecinoid which comprises the chemical structure of Formula B14, or functionalized or substituted variants thereof:

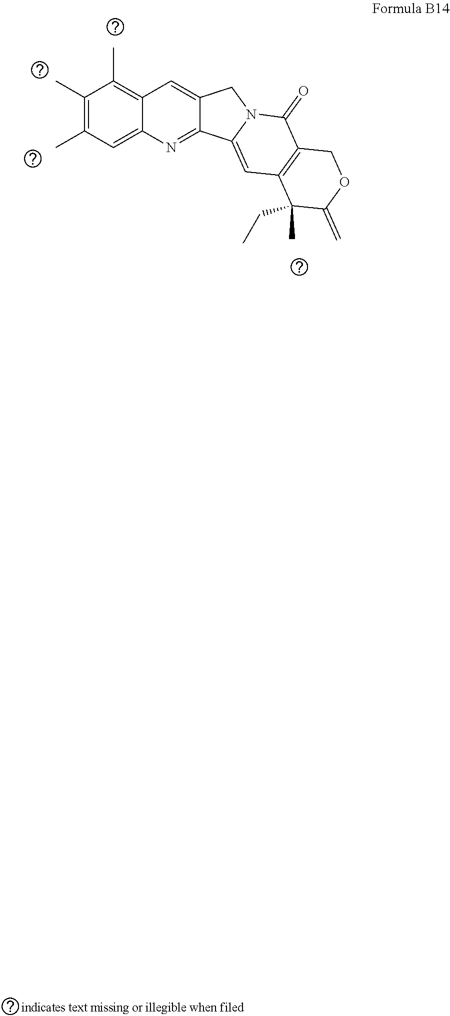 [SMILES: C=C1OCc2c(cc3n(c2=O)Cc2cc4c(C)c(C)c(C)cc4nc2-3)[C@@]1(C)CC]

For example the hydroxylated camptothecinoid may be a 9-X-10-hydroxycamptothecin (compound 11 in Table 5), X-11-hydroxycamptothecin (compound 14 in Table 5), X-10-hydroxycamptothecin or X-9-hydroxycamptothecin.

Furthermore, the camptothecinoid product of the catalytic reaction may be for example 9-hydroxycamptothecinoid, 9,10-dihydroxycamptothecinoid, 10-hydroxycamptothecinoid, 11-hydroxycamptothecinoid, 10,11-dihydroxycamptothecinoid or 9,11-dihydroxycamptothecinoid, 7-ethyl-9-hydoxycamptothecinoid, 7-ethyl-10-hydoxycamptothecinoid, 7-ethyl-11-hydoxycamptothecinoid, 7-ethyl-9,10-dihydoxycamptothecinoid, 7-ethyl-9,11-dihydoxycamptothecinoid, 7-ethyl-10,11-dihydoxycamptothecinoid, 9-amino-10-hydroxycamptothecinoid, 9-amino-11-hydroxycamptothecinoid, 9-amino-10,11-dihydroxycamptothecinoid, 10-hydroxy-11-methoxycamptothecin, 11-hydroxy-10-methoxycamptothecin.

In an embodiment the camptothecinoid product may be for example 9-hydroxycamptothecin, 9,10-dihydroxycamptothecin, 10-hydroxycamptothecin, 11-hydroxycamptothecin, 10,11-dihydroxycamptothecin or 9,11-dihydroxycamptothecin, 7-ethyl-9-hydoxycamptothecin, 7-ethyl-10-hydoxycamptothecin, 7-ethyl-11-hydoxycamptothecin, 7-ethyl-9,10-dihydoxycamptothecin, 7-ethyl-9,11-dihydoxycamptothecin, 7-ethyl-10,11-dihydoxycamptothecin, 9-amino-10-hydroxycamptothecin, 9-amino-11-hydroxycamptothecin, 9-amino-10,11-dihydroxycamptothecin, 10-hydroxy-11-methoxycamptothecin, 11-hydroxy-10-methoxycamptothecinIn a preferred embodiment the camptothecinoid product is 9-hydroxycamptothecin, 10-hydroxycamptothecin, 7-ethyl-10-hydroxycamptothecin, 9-amino-10-hydroxycamptothecin, 11-hydroxycamptothecin, 7-ethyl-11-hydroxycamptothecin, 9-amino-11-hydroxycamptothecin or 10,11-dihydoxycamptothecin.

### Hydroxylated Evodiaminoid

The evodiaminoid product of the catalytic reaction may be a hydroxylated evodiaminoid.

The hydroxylated evodiaminoid may be 9-hydroxy-evodiaminoid which comprises the chemical structure of Formula D1, or functionalized or substituted variants thereof:

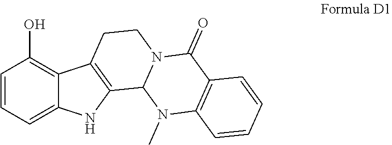 [SMILES: CN1c2ccccc2C(=O)N2CCc3c([nH]c4cccc(O)c34)C21]

The hydroxylated evodiaminoid may be 10-hydroxy-evodiaminoid which comprises the chemical structure of Formula D2, or functionalized or substituted variants thereof:

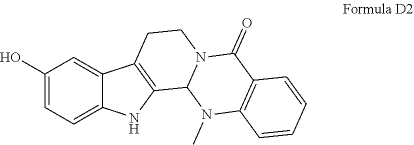 [SMILES: CN1c2ccccc2C(=O)N2CCc3c([nH]c4ccc(O)cc34)C21]

The hydroxylated evodiaminoid may be 11-hydroxy-evodiaminoid which comprises the chemical structure of Formula D3, or functionalized or substituted variants thereof:

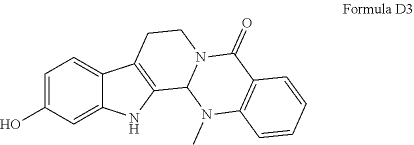 [SMILES: CN1c2ccccc2C(=O)N2CCc3c([nH]c4cc(O)ccc34)C21]

The hydroxylated evodiaminoid may be 10,11-dihydroxy-evodiaminoid which comprises the chemical structure of Formula D4, or functionalized or substituted variants thereof:

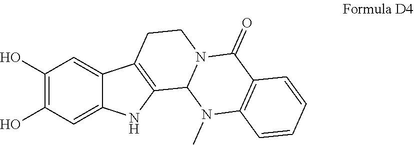 [SMILES: CN1c2ccccc2C(=O)N2CCc3c([nH]c4cc(O)c(O)cc34)C21]

The hydroxylated evodiaminoid product may for example be 9-hydroxyevodiaminoid, 9,10-hydroxy evodiaminoid, 10-hydroxy evodiaminoid, 11-hydroxy evodiaminoid, 10,11 -dihydroxy evodiaminoid or 9,11-dihydroxy evodiaminoid.

Accordingly, non-limiting products produced by the current method and process may include 9-hydroxy-evodiaminoid, 9,10-dihydroxyevodiaminoid, 10-hydroxy-evodiaminoid, 11-hydroxy evodiaminoid, 10,11-dihydroxy evodiaminoid or 9,11-dihydroxyevodiaminoid.

For example, the products may include 9-hydroxy-evodiamine, 9,10-dihydroxy-evodiamine, 10-hydroxy-evodiamine, 11-hydroxy-evodiamine, 10,11-dihydroxy-evodiamine, 9,11-dihydroxy-evodiamine, 13b-hydroxy evodiaminoid, 9,13b-dihydroxy evodiaminoid, 10,13b-dihydroxy evodiaminoid, or 11,13b-dihydroxy evodiaminoid.

### Hydroxylated Ellipticinoid

The ellipticinoid product of the catalytic reaction may be a hydroxylated ellipticinoid.

The hydroxylated ellipticinoid may be 8-hydroxy-ellipticinoid which comprises the chemical structure of Formula E1, or functionalized or substituted variants thereof:

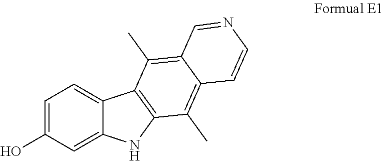 [SMILES: Cc1c2ccncc2c(C)c2c1[nH]c1cc(O)ccc12]

The hydroxylated ellipticinoid may be 9-hydroxy-ellipticinoid which comprises the chemical structure of Formula E2, or functionalized or substituted variants thereof:

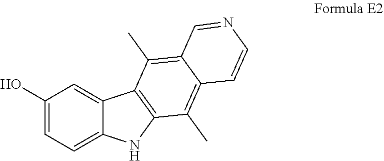 [SMILES: Cc1c2ccncc2c(C)c2c1[nH]c1ccc(O)cc12]

The hydroxylated ellipticinoid may be 10-hydroxy-ellipticinoid which comprises the chemical structure of Formula E3, or functionalized or substituted variants thereof:

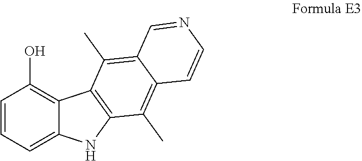 [SMILES: Cc1c2ccncc2c(C)c2c1[nH]c1cccc(O)c12]

The hydroxylated ellipticinoid may be 8,9-dihydroxy-ellipticinoid which comprises the chemical structure of Formula E4, or functionalized or substituted variants thereof:

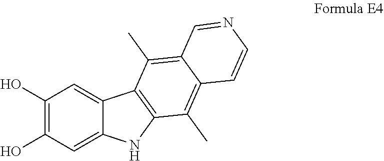 [SMILES: Cc1c2ccncc2c(C)c2c1[nH]c1cc(O)c(O)cc12]

The hydroxylated ellipticinoid product may be for example 9-hydroxy-ellipticinoid, 9,10-hydroxyevodiaminoid, 8-hydroxy-ellipticinoid, 10-hydroxy-ellipticinoid, 7-hydroxy-ellipticine, 12-hydroxy-ellipticine, 13-hydroxy-ellipticine, 8,9-dihydroxy-ellipticinoid, 9,10-hydroxy-ellipticinoid, 8,10-dihydroxy-ellipticinoid.

Accordingly, non-limiting products produced by the current method and process may include 8-hydroxy-ellipticinoid, 9-hydroxy-ellipticinoid, 10-hydroxy-ellipticinoid, 7-hydroxy-ellipticine, 12-hydroxy-ellipticine, 13-hydroxy-ellipticine, 9,10-dihydroxy-ellipticinoid, 8,9-dihydroxy-ellipticinoid, 8,10-dihydroxy-ellipticinoid. Furthermore, the non-limiting products may include 8-hydroxy-ellipticine, 9-hydroxy-ellipticine, 10-hydroxy-ellipticine, 9,10-dihydroxy ellipticine, 8,9-dihydroxy ellipticine, 8,10-dihydroxy ellipticine.

Non-limiting examples of hydroxylated MIA or dihydroxylated MIA that may be produced by the disclosed method or process are also listed in Table 4A and 4B.

### Monoterpenoid Indole Alkaloid (MIA) Derivatives

In a further aspect, the present disclosure relates to MIA product derivative (also referred to as MIA product derivatives or hydroxylated MIA derivatives) that may be derived from the MIA product by further reacting the MIA product, for example the camptothecinoid product, the evodiaminoid product or the ellipticinoid product. Methods and processes of making such MIA product derivatives are also provided.

As described above, the production of the MIA products comprises contacting of a MIA substrate with the cytochrome P450 monooxygenase under conditions suitable for oxidation or hydroxylation of the MIA substrate, thereby forming a MIA product.

The MIA product may be isolated, recovered, extracted or purified using known and conventional methods within the art. The recovered or purified MIA product may then be further reacted to yield MIA product derivatives or hydroxylated MIA derivatives, the MIA product derivative may for example be a camptothecinoid derivative, an evodiaminoid derivative or an ellipticinoid derivative.

The production of MIA product derivatives from the MIA products produced through the methods and processes described herewith may be done through conventional chemical reactions that are well known within the art.

In certain embodiments, the MIA derivatives may be camptothecine (CPT) derivative. As used herein, a “CPT derivative” refers to any compound known in the art for which CPT is a precursor for synthesis. The CPT derivative may be a direct or indirect synthesis product of CPT. Synthesis of the CPT derivative may occur in vivo or in vitro, by the method or process as described herewith.

For example the synthesis of MIA product derivatives may occur through reaction of the MIA product such as a camptothecinoid product, an evodiaminoid product or a ellipticinoid product with a composition comprising a reagent.

For example, the reagent may be an iminium reagent, iminium salt, iminium catalyst, halogen reagent, halogenated reagent, or other another reagent known in the art. In some embodiments, the iminium reagent may be N,N-dimethylmethyleneiminium chloride, [1,4′]bipiperidinyl-1′-carbonyl chloride, or other iminium cations or salts known in the art, for example as described by Erkkila et al (Chem. Rev. 2007, 107, 12, 5416-5470) which is incorporated herein by reference. The halogenated reagent may be N-bromosuccinimide, thionyl chloride, N-chlorosuccinimide, phosphorus(V) oxychloride, N-iodosuccinimide, cyanuric chloride, tetrabromomethane, carbon tetrachloride, sulfuryl chloride, 1,3-dibromo-5,5-dimethylhydantoin, bromine, phosphorus(V) oxybromide, carbon tetrachloride, triphenylphosphine dibromide, phosphorus pentachloride, boron triiodide, thionyl bromide, sulfuryl chloride, methyltriphenoxyphosphonium iodide, phosphorus pentabromide, dibromoisocyanuric acid, iodine monochloride, iodine trichloride, phosphorus trichloride, phosphorus tribromide, B-iodo-9-BBN, iodine monochloride, B-chlorocatecholborane, iodine monochloride, phosphorus triiodide, benzyltrimethylammonium dichloroiodate, tetraiodomethane, 1,3,4,6-tetrachloro-3α,6α-diphenylglycouril, iodine monobromide, 1-[(triisopropylsilyl)ethynyl]-1,2-benziodoxol-3(1H)-one, iodine, tetrabutylammonium triiodide, triphenylphosphine diiodide, pyridinium tribromide, ethyl tribromoacetate, bromomethylenemorpholinium bromide, N-chloro-N-(1,1-dimethylethyl)-3,5-bis(trifluoromethyl)-benzamide, 2,3-dibromo-propylamine, bromodiethylsulfonium bromopentachloroantimonate(V), N,N-dimethyl-N-(methylsulfanylmethylene)ammonium iodide, bromodimethylsulfonium bromide, S-methyl N-(2,2,2-trichloroethoxysulfonyl)carbonchloroimidothioate, N-(2,2,2-trichloroethoxysulfonyl)urea, phosphorus tribromide, or 4-(dimethylamino)pyridine tribromide. For example the reagent may be N,N-dimethyl-methyleneiminum cation, 1-chlorocarbonyl-4-piperidinopiperidine hydrochloride, N-bromosuccinimide or N,N-dimethyl-methyleneiminum.

For example a hydroxylated camptothecinoid may be reacted with a composition comprising N-bromosuccinimide.

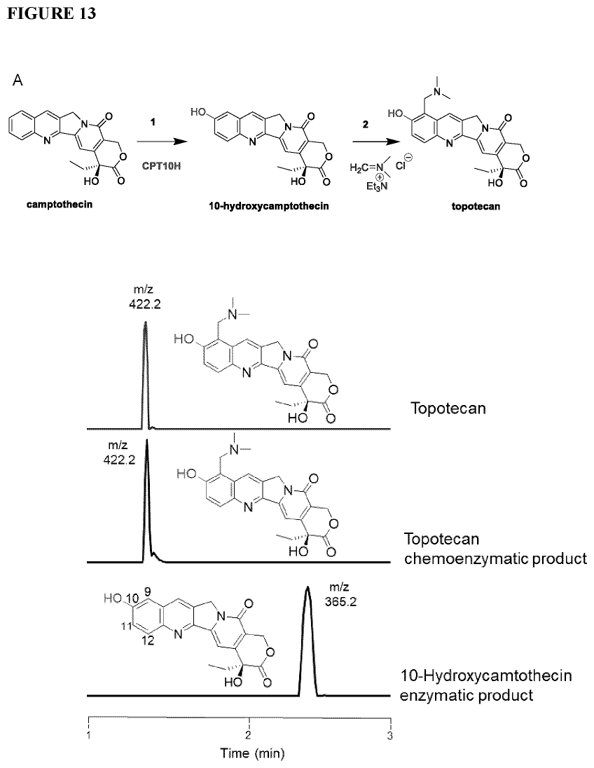

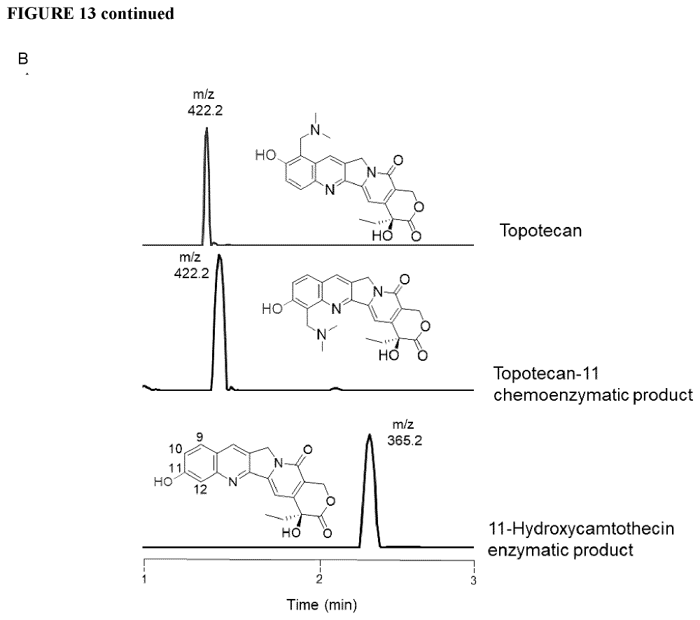

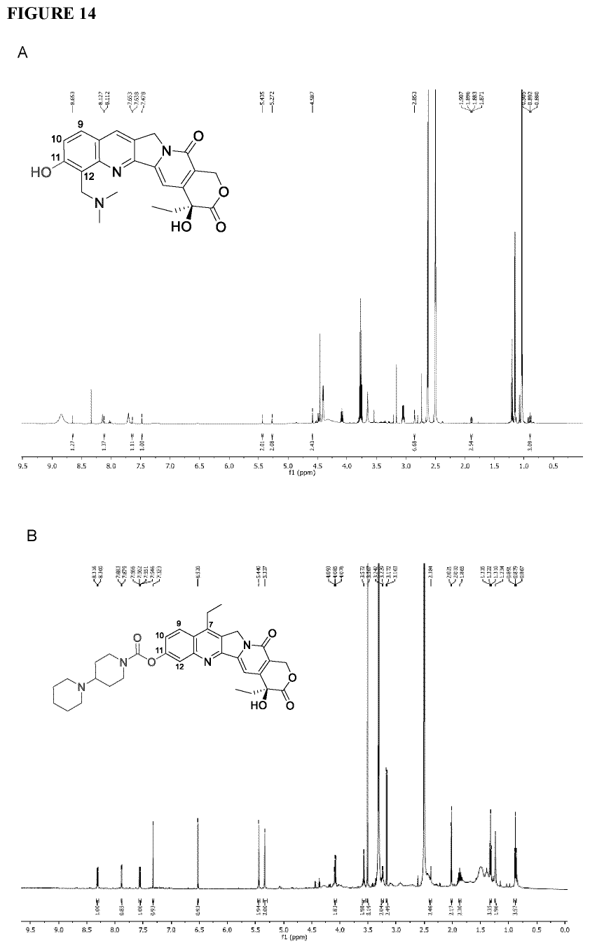

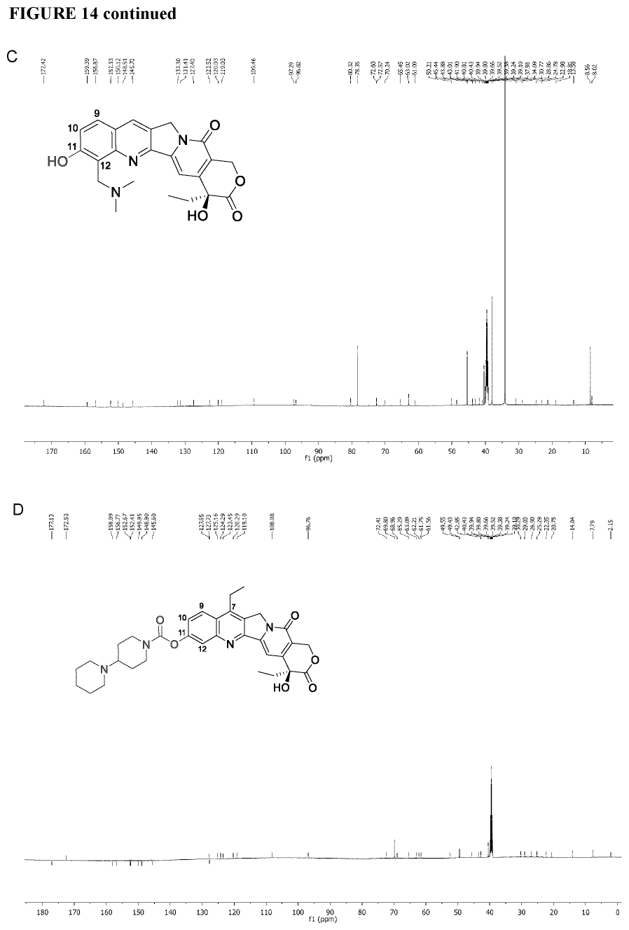

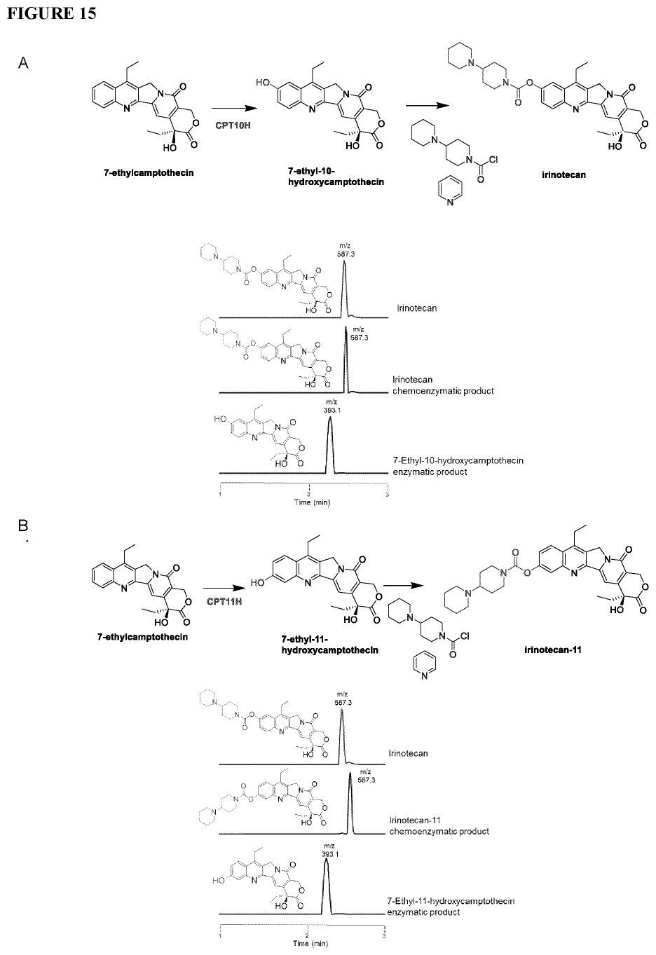

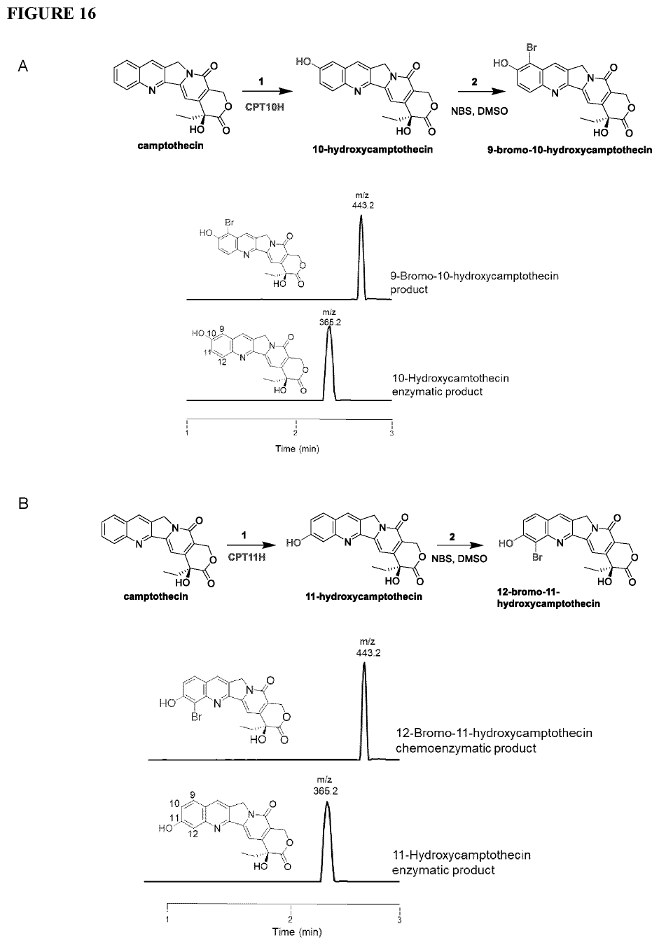

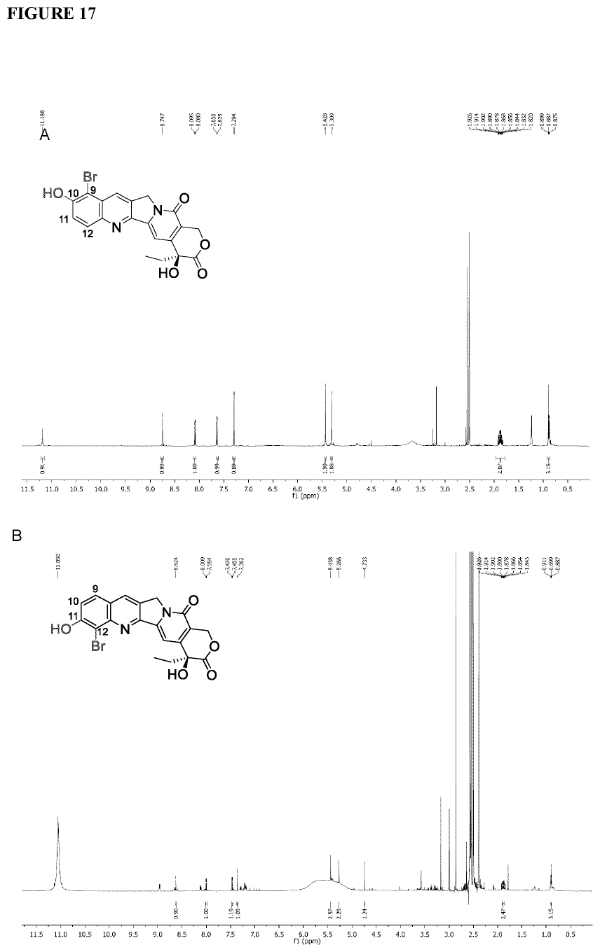

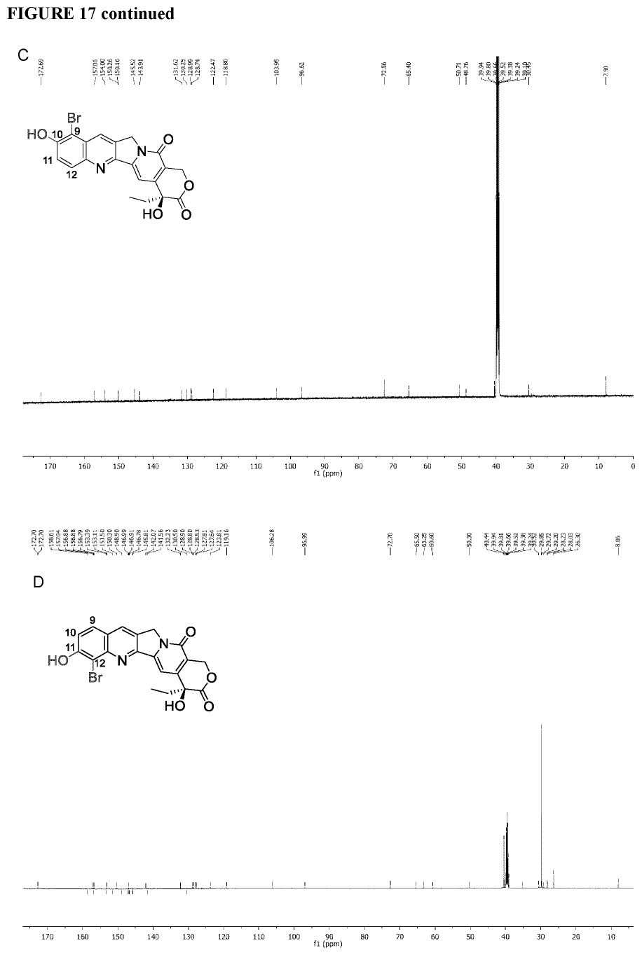

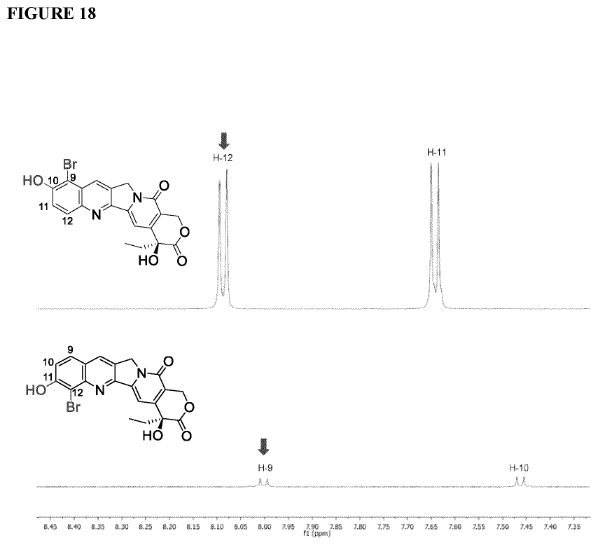

The following non-limiting examples are provided in the disclosure:
Enzymatic conversion of camptothecin to 10-hydroxycamptothecin, followed by treatment with an iminium reagent, N,N-dimethylmethyleneiminium chloride, yielding 9-[(dialkylamino)methyl]-10HCPT, commonly known as topotecan (FIG. 3A; FIG. 13A);
Enzymatic conversion of camptothecin to 11-hydroxycamptothecin, followed by treatment with an iminium reagent, N,N-dimethylmethyleneiminium chloride, yielding 12-[(dialkylamino)methyl]-11HCPT (topotecan-11) (FIGS. 3A, 13B and 14A);
Enzymatic conversion of camptothecin to 7-ethyl-10-hydroxycamptothecin (also called SN-38), followed by treatment with [1,4′]bipiperidinyl-1′-carbonyl chloride in pyridine, yielding 7-ethyl-10-[4-(1-piperidino)-1-piperidino]carbonyloxycamptothecin, commonly known as irinotecan (FIGS. 3B; 14B, and 15);
Enzymatic conversion of camptothecin to 7-ethyl-11-hydroxycamptothecin, followed by treatment with [1,4′]bipiperidinyl-1′-carbonyl chloride in pyridine, yielding 7-ethyl-11-[4-(1-piperidino)-1-piperidino]carbonyloxycamptothecin (irinotecan-11) (FIGS. 3B; 14B, and 15);
Enzymatic conversion of camptothecin to 10-hydroxycamptothecin, followed by treatment with a halogenated reagent, yielding 9-halo-10-hydroxycamptothecin. For example, the present disclosure provides for the enzymatic conversion of camptothecin to 10-hydroxycamptothecin, followed by treatment with N-bromosuccinimide, yielding 9-bromo-10-hydroxycamptothecin (FIGS. 16, 17 and 18);
Enzymatic conversion of camptothecin to 11-hydroxycamptothecin, followed by treatment with a halogenated reagent, yielding 12-halo-11-hydroxycamptothecin. For example Camptothecin was converted to 11-hydroxy-camptothecin, followed by treatment with N-bromosuccinimide, yielding 12-bromo-11-hydroxy-camptothecin (FIGS. 16, 17 and 18).
Enzymatic conversion of camptothecin to 10-hydroxycamptothecin, which is further reacted to topotecan (FIGS. 1, 3A, and 13A and Example 6);
Enzymatic conversion of 7-ethyl camptothecin to 7-ethyl-10-hydroxycamptothecin, which is further reacted to irinotecan (see FIGS. 3B and 15A and Example 6);
Enzymatic conversion of camptothecin to 10-hydroxycamptothecin, which is further reacted to form 9-bromo-10-hydroxycamptothecin. Similar methods may be used to produce analogous 9-halo-10-hydroxycamptothecin compounds (see FIG. 16A and Example 6);
Enzymatic conversion of camptothecin to 11-hydroxycamptothecin, which is further reacted to form topotecan-11 (FIGS. 1, 3A, and Example 6);
Enzymatic conversion of 7-ethylcamptothecin to 7-ethyl-ll-hydroxycamptothecin, which is further reacted to form irinotecan-11 (FIGS. 3B and 15B and Example 6);
Enzymatic conversion of camptothecin to 11-hydroxycamptothecin, which is further reacted to form 12-bromo-11-hydroxycamptothecin. Similar methods may be used to produce analogous 12-halo-11-hydroxycamptothecin compounds (FIG. 16B and Example 6).

In one embodiment, the present disclosure may provide a method of making topotecan, the method comprising
(i) contacting a host or host cell comprising a recombinant cytochrome P450 monooxygenase enzymes as described herewith with camptothecin;
(ii) growing the host or host cell under conditions suitable for the production of 10-hydroxycamptothecin;
(iii) optionally, isolating the 10-hydroxycamptothecin formed in step (ii);
(iv) subsequently reacting the 10-hydroxycamptothecin with N,N-dimethyl-methyleneiminum cation to produce topotecan.

In a further embodiment, the present diclosure may provide a method of making 7-ethyl-10-hydoxycamptothecin (SN-38), the method comprising contacting a host or host cell comprising a recombinant recombinant cytochrome P450 monooxygenase enzymes as described herewith with 7-ethyl-camptothecin;
(ii) growing the host cell under conditions suitable for the production of 7-ethyl-10-hydoxycamptothecin;
(iii) optionally, isolating the 7-ethyl-10-hydoxycamptothecin formed in step (ii).

In another embodiment, the present disclosure may provide a method of making irinotecan, the method comprising
(i) contacting a host or host cell comprising a recombinant cytochrome P450 monooxygenase enzymes as described herewith with 7-ethyl-camptothecin;
(ii) growing the host or host cell under conditions suitable for increased production of 7-ethyl-10-hydroxycamptothecin;
(iii) optionally, isolating the 7-ethyl-10-hydroxycamptothecin formed in step (ii);
(iv) subsequently reacting the 7-ethyl-10-hydroxycamptothecin with 1-chlorocarbonyl-4-piperidinopiperidine hydrochloride to produce irinotecan.

In one embodiment, the present disclosure may provide a method of making topotecan, the method comprising
(i) contacting a host or host cell comprising a recombinant recombinant cytochrome P450 monooxygenase enzymes as described herewith with camptothecin;
(ii) growing the host or host cell under conditions suitable for production of 10-hydroxycamptothecin;
(iii) optionally, isolating the 10-hydroxycamptothecin formed in step (ii);
(iv) subsequently reacting the 10-hydroxycamptothecin with reagents such as N-bromosuccinimide to produce 9-bromo-10-hydroxycamptothecin.

In one the present disclosure may provide a method of making topotecan 11-hydroxy isomer, the method comprising
(i) contacting a host or host cell comprising a recombinant cytochrome P450 monooxygenase enzymes as described herewith with camptothecin;
(ii) growing the host or host cell under conditions suitable for production of 10-hydroxycamptothecin;

(iii) optionally, isolating the 11-hydroxycamptothecin formed in step (ii);

(iv) subsequently reacting the 11-hydroxycamptothecin with N,N-dimethyl-methyleneiminum cation to produce topotecan.

In further embodiment, the present disclosure may provide a method of making 7-ethyl-11-hydoxycamptothecin, the method comprising contacting a host or host cell comprising a recombinant cytochrome P450 monooxygenase enzymes as described herewith with 7-ethyl-camptothecin;
(ii) growing the host or host cell under conditions suitable for production of 7-ethyl-11-hydoxycamptothecin;
(iii) optionally, isolating the 7-ethyl-11-hydoxycamptothecin formed in step (ii).

In one embodiment, the present disclosure may provide a method of making irinotecan 11-hydroxy isomer, the method comprising
(i) contacting a host or host cell comprising a recombinant cytochrome P450 monooxygenase enzymes as described herewith with 7-ethyl-camptothecin;
(ii) growing the host or host cell under conditions suitable for production of 7-ethyl-11-hydroxycamptothecin;
(iii) optionally, isolating the 7-ethyl-11-hydroxycamptothecin formed in step (ii);
(iv) subsequently reacting the 7-ethyl-11-hydroxycamptothecin with 1-chlorocarbonyl-4-piperidinopiperidine hydrochloride to produce irinotecan.

In one embodiment, the present disclosure may provide a method of making irinotecan 11-hydroxy isomer, the method comprising
(i) contacting a host cell comprising a recombinant cytochrome P450 monooxygenase enzymes as described herewith with 10-hydroxycamptothecin;
(ii) growing the host cell under conditions suitable for production of 10,11-dihydroxycamptothecin;
(iii) optionally, isolating the 10,11-dihydroxycamptothecin formed in step (ii);
(iv) subsequently reacting the 10,11-dihydroxycamptothecin with 1-chlorocarbonyl-4-piperidinopiperidine hydrochloride to produce irinotecan.

In one embodiment, the present disclosure may provide a method of making topotecan, the method comprising
(i) contacting a host or host cell comprising a recombinant cytochrome P450 monooxygenase enzymes with camptothecin;
(ii) growing the host or host cell under conditions suitable for production of 11-hydroxycamptothecin;
(iii) optionally, isolating the 11-hydroxycamptothecin formed in step (ii);
(iv) subsequently reacting the 11-hydroxycamptothecin with reagent such as N-bromosuccinimide to produce bromo-11-hydroxycamptothecin.

Non-limiting examples of camptothecin derivatives that may be synthesized through treatment of the hydroxylated camptothecinoid with a composition comprising a reagent are also listed in Table 4B.

For example the MIA products may be 10-hydroxycamptothecin, 7-ethyl-10-hydroxycamptothecin, 11-hydroxycamptothecin, or 7-ethyl-11-hydroxycamptothecin, which optionally may be converted through a subsequent reaction to MIA derivatives or analogues such as for example camptothecin derivatives or analogues. The camptothecin derivatives may be, for example, 10-hydroxycamptothecin (2), 11-hydroxycamptothecin (10), 7-ethyl-10-hydroxy-camptothecin (7), 7-ethyl-11-hydroxycamptothecin (8), 10,11-dihydroxycamptothecin (12), 9-amino-10-hydroxycamptothecin (18), 9-amino-11-hydroxycamptothecin (19), topotecan (4), 12-[(dimethylamino)methyl]-11-hydroxycamptothecin (9), 9-bromo-10-hydroxycamptothecin (11a), 12-bromo-11-hydroxycamptothecin (17), Irinotecan-11 (10), irinotecan (3) (see for example Table 5).

The MIA product derivative may further be a evodiaminoid derivative as described in CN105418610, which is herein incorporated by reference. For example the following R groups may be generated from a 10-hydroxyevodiamine product produced by the present method or process: trifluoromethyl, trifluoromethoxy, methoxyl group, oxyethyl group, propoxy-, isopropoxy or butoxy; Lower hydroxy alkyl, low-grade alkyl amino, low-grade halogenated alkyl are amino, low-grade cycloalkyl is amino, alkynyl of low-grade chain is amino, amide group, low-grade cycloalkyl amide group, rudimentary amido alkyl; with Boc and the amino acid sloughing Boc; hydrogen, halogen, low-grade halogenated alkyl, low alkyl group, hydroxyl, Lower hydroxy alkyl, lower alkoxy, amino, low-grade alkyl amino, low-grade halogenated alkyl are amino, low-grade cycloalkyl is amino, alkynyl of low-grade chain is amino, amide group, low-grade cycloalkyl amide group, rudimentary amido alkyl.

The camptothecin derivative may be a topoisomerase I inhibitor. As used herein, a “topoisomerase I inhibitor” refers to a class of anticancer agents which interrupt DNA replication in cancer cells, the result of which is cell death. Most if not all topoisomerase I inhibitors are derivatives of camptothecin.

### Camptothecin Derivatives and Analogues

The present disclosure also provides derivatives or analogues of camptothecin and the process of their preparation, to their use as active ingredients for the preparation of medicament useful in the treatment of tumors, and to pharmaceutical preparations containing them.

In one aspect the present disclosure relates to a compound of formula I.

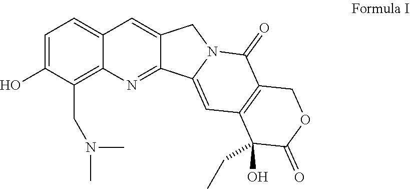 [SMILES: CC[C@@]1(O)C(=O)OCc2c1cc1n(c2=O)Cc2cc3ccc(O)c(CN(C)C)c3nc2-1]

The compound of Formula I may also be referred to as 12-[(dimethylamino)methyl]-11-hydroxycamptothecin (topotecan-11, also refered to as [12-[(dialkylamino)methyl]-11HCPT). The compound may be produced by the method or process as described herein.

In a further aspect the present disclosure also provides for a compound of formula II:

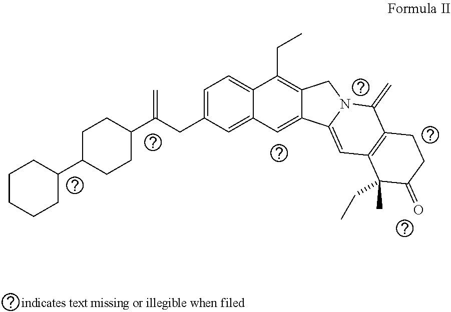 [SMILES: C=C(Cc1ccc2c(CC)c3c(cc2c1)C1=CC2=C(CCC(=O)[C@]2(C)CC)C(=C)N1C3)C1CCC(C2CCCCC2)CC1]

The compound of Formula II may also be referred to as 7-ethyl-11-[4-(1-piperidino)-1-piperidino]carbonyloxycamptothecin (irinotecan-11). The compound may be produced by the method or process as described herein.

In another aspect the present disclosure provides for a compound of formula III:

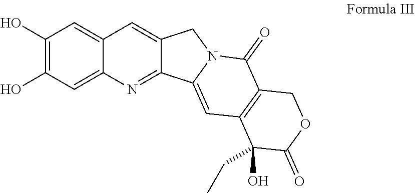 [SMILES: CC[C@@]1(O)C(=O)OCc2c1cc1n(c2=O)Cc2cc3cc(O)c(O)cc3nc2-1]

The compound of Formula III may also be referred to as 10,11-dihydroxy-CPT. The compound may be produced by the method or process as described herein.

In a further aspect it is provided a compound of formula IV:

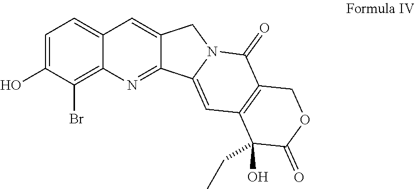 [SMILES: CC[C@@]1(O)C(=O)OCc2c1cc1n(c2=O)Cc2cc3ccc(O)c(Br)c3nc2-1]

The compound of Formula IV may also be referred to as 12-bromo-11HCPT. The compound may be produced by the method or process as described herein.

In a another aspect it is provided a compound of formula V:

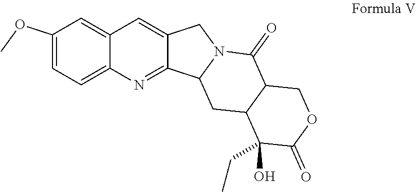 [SMILES: CC[C@@]1(O)C(=O)OCC2C(=O)N3Cc4cc5cc(OC)ccc5nc4C3CC21]

The compound of Formula V may also be referred to as 10-hydroxy-11-methoxycamptothecin. The compound may be produced by the method or process as described herein.

In a further aspect it is provided a compound of formula VI:

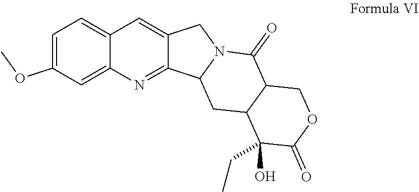 [SMILES: CC[C@@]1(O)C(=O)OCC2C(=O)N3Cc4cc5ccc(OC)cc5nc4C3CC21]

The compound of Formula VI may also be referred to as 11-hydroxy-10-methoxycamptothecin. The compound may be produced by the method or process as described herein.

In a another aspect it is provided a compound of formula VII:

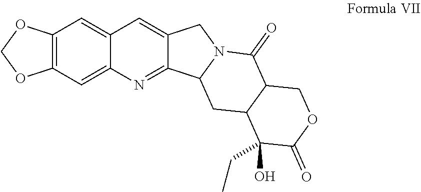 [SMILES: CC[C@@]1(O)C(=O)OCC2C(=O)N3Cc4cc5cc6c(cc5nc4C3CC21)OCO6]

The derivatives or analogues of camptothecin may exhibit a potent antiproliferative activity and may possess physico-chemical properties that make them suitable to be included in pharmaceutically acceptable compositions.

Pharmaceutically acceptable salts of compounds of formula (I) to (VII) can be obtained according to literature methods.

In another aspect it is therefore provided pharmaceutical composition comprising camptothecin derivatives or analogues as described herewith.

The present disclosure is further directed to pharmaceutical compositions containing an effective amount of at least a compound of formula I, II, III, IV, V, VI or VII as active ingredient in admixture with vehicles and excipients. Pharmaceutical compositions may be prepared according to conventional methods well known in the art, for example as described in Remington's Pharmaceutical Sciences Handbook, Mack. Pub., N.Y., U.S.A.

Examples of pharmaceutical compositions are injectable compositions, such as solutions, suspensions emulsions in aqueous or non aqueous vehicle; enteral composition, such as capsules, tablets, pills, syrups, drinkable liquid formulations. Other pharmaceutical compositions compatible with the compounds of formula I I, II, III, IV, V, VI or VII are controlled release formulations.

The dosage of the active ingredient in the pharmaceutical composition shall be determined by the person skilled in the art depending on the activity and pharmacokinetic characteristics of the active ingredient. The posology shall be decided by the physician on the grounds of the type of tumor to be treated, and the conditions of the patient. The compounds of the present disclosure may also be used in combination therapy with other antitumor drugs.

It is further provided a method of treating cancer in a subject in need thereof with the pharmaceutical composition and/or camptothecin derivative or analogues as described herewith. The cancer treated in the subject may for example be lung, cervix, ovarian or colon cancers.

In another aspect it is therefore provided a method of using the camptothecin derivative or analogues of the present disclosure and/or pharmaceutical composition comprising the same as a palliative to ameliorate one or more of the symptoms associated with cancer, which comprises administering to a subject in need thereof an effective amount of the camptothecin derivative or analogues of the present disclosure and/or a pharmaceutical composition comprising the same. The amelioration of symptoms associated with cancer may improve the quality of life for patients with cancer, such for example lung, cervix, ovarian or colon cancers.

The camptothecin derivative or analogues of the present disclosure and/or pharmaceutical composition may be used in single agent therapy for any of the above-described treatments or uses or may be used in combination with other active treatment modalities such as radiation therapy, conventional anti-neoplastic agents, which include but are not limited to paclitaxel, docetaxel, doxorubicin, ara-c (cytarabine), 5-fluorouracil, etoposide and organometallic coordination compounds, such as cisplatin and carboplatin and targeted biologic therapeutic approaches, which include but are not limited to, gefitinib, erlotinib, lapatinib, bortezimib, elacridar, and erbitux.

The term “effective amount” means that amount of the camptothecin derivative or analogues of the present disclosure and/or pharmaceutical composition containing the same, that upon administration to a mammal (such as a human being), in need thereof, provides a clinically desirable result in the treatment of various diseases, i.e., such as virally-related and/or cancer diseases (i.e., the latter of which may include anti-neoplastic treatment, which includes, but not limited to, tumor cell growth inhibition, remission, cure, amelioration of symptoms, etc.).

Further provided is a kit comprising a vector (as described above) or a host or host cell (as described above), in combination with instructions for producing a MIA products as described above.

The disclosure further provides the following sequences.

TABLE 2
SEQ ID NOs and Description of Sequences

| SEQ | |
|---|---|
| ID | |
| NO: | Description of Sequence |
| 1 | Coding nucleotide sequence of camptothecin hydroxylase Ca32236/CPT10H from C. acuminata |
| 2 | Coding nucleotide sequence of camptothecin hydroxylase Ca32229/CPT11H from C. acuminata |
| 3 | Amino acid sequence of camptothecin hydroxylase Ca32236/CPT10H from C. acuminata |
| 4 | Amino acid sequence of camptothecin hydroxylase Ca32229/CPT11H from C. acuminata |
| 5 | Coding nucleotide sequence of putative camptothecin hydroxylase CPT10H ortholog 1 from Ophiorrhiza |
| | pumila |
| 6 | Coding nucleotide sequence of putative camptothecin hydroxylase CPT10H ortholog 2 from Ophiorrhiza |
| | pumila |
| 7 | Coding nucleotide sequence of putative camptothecin hydroxylase CPT10H ortholog 3 from Ophiorrhiza |
| | pumila |
| 8 | Amino acid sequence of putative camptothecin hydroxylase CPT10H ortholog 1 from Ophiorrhiza pumila |
| 9 | Amino acid sequence of putative camptothecin hydroxylase CPT10H ortholog 2 from Ophiorrhiza pumila |
| 10 | Amino acid sequence of putative camptothecin hydroxylase CPT10H ortholog 3 from Ophiorrhiza pumila |
| 11 | Coding nucleotide sequence of putative camptothecin hydroxylase CPT11H ortholog 1 from Ophiorrhiza |
| | pumila |
| 12 | Coding nucleotide sequence of putative camptothecin hydroxylase CPT11H ortholog 2 from Ophiorrhiza |
| | pumila |
| 13 | Coding nucleotide sequence of putative camptothecin hydroxylase CPT11H ortholog 3 from Ophiorrhiza |
| | pumila |
| 14 | Amino acid sequence of putative camptothecin hydroxylase CPT11H ortholog 1 from Ophiorrhiza pumila |
| 15 | Amino acid sequence of putative camptothecin hydroxylase CPT11H ortholog 2 from Ophiorrhiza pumila |
| 16 | Amino acid sequence of putative camptothecin hydroxylase CPT11H ortholog 3 from Ophiorrhiza pumila |
| 17 | Coding nucleotide sequence of putative camptothecin hydroxylase CPT10H ortholog from N. nimmoniana |
| 18 | Amino acid sequence of putative camptothecin hydroxylase CPT10H ortholog from N. nimmoniana |
| 19 | Coding nucleotide sequence of putative camptothecin hydroxylase CPT11H ortholog from N. nimmoniana |
| 20 | Amino acid sequence of putative camptothecin hydroxylase CPT11H ortholog from N. nimmoniana |
| 21 | Coding nucleotide sequence of putative camptothecin hydroxylase Ca6009 from Camptotheca acuminata |
| 22 | Amino acid sequence of putative camptothecin hydroxylase Ca6009 from Camptotheca acuminata |
| 23 | Coding nucleotide sequence of putative camptothecin hydroxylase Ca6007 from Camptotheca acuminata |
| 24 | Amino acid sequence of putative camptothecin hydroxylase Ca6007 from Camptotheca acuminata |
| 25 | Coding nucleotide sequence of putative camptothecin hydroxylase Ca23831/CPT9H from C. acuminata |
| 26 | Amino acid sequence of putative camptothecin hydroxylase Ca23831/CPT9H from C. acuminata |
| 27 | Coding nucleotide sequence of putative camptothecin hydroxylase Ca23838 from Camptotheca acuminata |
| 28 | Amino acid sequence of putative camptothecin hydroxylase Ca23838 from Camptotheca acuminata |
| 29 | Coding nucleotide sequence of putative camptothecin hydroxylase Ca32245 from Camptotheca acuminata |
| 30 | Amino acid sequence of putative camptothecin hydroxylase Ca32245 from Camptotheca acuminata |
| 31 | Primer sequence of pESC-Leu2d-32245 F |
| 32 | Primer sequence of pESC-Leu2d-32245 R |
| 33 | Primer sequence of pESC-Leu2d-32236 F |
| 34 | Primer sequence of pESC-Leu2d-32236 R |
| 35 | Primer sequence of pESC-Leu2d-32245 F |
| 36 | Primer sequence of pESC-Leu2d-32245 R |
| 37 | Primer sequence of pESC-Leu2d-32229 F |
| 38 | Primer sequence of pESC-Leu2d-32229 R |

The present following examples are provided:

### EXAMPLES

CPT is a powerful but not ideal anticancer drug owing to its low solubility, undesirable side effects and drug resistance 13. Chemical substitutions on the CPT scaffold are thus required to improve its potency. Hydroxylations at C-10 and C-11 on ring A of the CPT scaffold are critical features in designing active CPT derivatives, with the semi-synthetic 10HCPT serving as the precursor for the commercial synthesis of the anticancer drugs topotecan and irinotecan. Although selective functionalization of unactivated C(sp³)-H bonds in natural products is especially chemically challenging due to their inherent complexity with various chiral centres and functional groups, natural selection provides elegant enzymatic tools that can help overcome these hurdles. Of these, CYP450s stand out as key and tractable biocatalysts with an ability to activate C—H bonds via oxidation with striking chemo-, regio- and stereo-selectivities. The ability of the newly-discovered CYP450-based CPT hydroxylases to oxidize a variety of CPT-derived scaffolds allowed to employ a chemoenzymatic pipeline leading to potent anti-tumour CPT derivatives. Importantly, the new enzymatic product 11HCPT and its derivatives described herewith have been known to exhibit a much greater therapeutic index with less toxicity than CPT46, with 11HCPT derivatives such as 7-ethyl-11HCPT overcoming interpatient variability and drug resistance compared with irinotecan.

The rising need for anticancer CPT derivatives requires more sustainable and direct chemoenzymatic steps starting from CPT at mild conditions (pH 7, 30° C.) as compared to chemical synthesis. The high regio-selectivity (for the C-10 and C-11 positions) and conversion rate (62%-67%) of CPT hydroxylases afford the production of specific HCPTs and derivatives with chemical decorations at desired positions. Among the new chemoenzymatic products, the bromo-CPT derivatives are of significant note. Halogenated organic compounds are scarce in nature, yet they constitute up to 15% of the pharmaceutical products on the market, and the bromo-HCPTs produced in this disclosure may provide starting handles for selective arylation via cross-coupling to further diversify the CPT-derived products with new bioactivity potentials.

More than half a century since the isolation of CPT from C. acuminata and forty years after the first report on HCPT chemical synthesis, the discovery and application of CPT hydroxylases in this disclosure open another window into the largely elusive CPT metabolism. It also represents a greener alternative to chemical semisynthesis of CPT derivatives and a significant expansion of the CPT chemical space, paving the way for the further regioselective functionalization of the rigid polycyclic alkaloid structures with new bioactive molecules.

### Example 1: Sequences

The following sequences are provided.

Coding nucleotide sequence of camptothecin hydroxylase Ca32236/CPT10H from
Camptotheca acuminata
SEQ ID NO: 1
ATGGAGAACTTGTACTACTGCCTTGCTCTCCTACTATCAATTCTTTTCATATTCAGACATTTCTTCCGTCATAGT
TCAAAGTTACCACCAAGTCCGTTTGCCCTTCCTATCATCGGCCATCTCCATCTCATCAGGAATTCTTTGCACCAA
GTACTAGAGTGCTTGGCATCTCAATATGGTCCAATTTTATTTCTCAAATTTGGCACCCGCTCTATTCTTGTTGTG
TCTTCTCCATCCGCTGTTGAGGAATGCCTCATTAAGAATGATATTATATTTGCAAACCGTCCTCGGAGCATGATT
TTAGATCTCTCTAGTTTTAATTATAGTATATTTTCATGGGCTCCATATGGTCATTACTGGCGAAGCCTCCGCCGC
CTTGCTGTTGTTGAACTCTTCACATCGCGCAGCCTTCAGACGTCTTCCAACATCCGTAAAGAGGAAATTCATAAC
CTTCTCTGTCACCTCTTCAAATTCTCAAAAAGTGGAACTCAAAAACTCCAGTTGAAATATTGGTTCTCTCTATTG
ACATTCAATATTATAACAAGGCTGGTAGCTGGGAAGCGGTGTGTTAGAGATGCAGTTGCAGGCACGGATTCGGGT
AAACAAATTCTTGAAGACCTCGAGGGGAAGTTCACTTCAAAAATGCCATTTAATATGTGTGATTTCTTTCCAATT
TTGAGGTGGTTTGGTTACAAAGGGTTGGAGAAAATTCTGATTACGTTGCACAAGGAGAGAGATGAATTCATGCAA
GGTTTGATAGATGAGGTTAGACGAAAGAGAACATGTTCTGCCAATATCAATAGTGTAACAAACAGAGCAAAGACA
ACATTGATTGAAGCTCTCTTGTCCCTCCAAGAATCAGAACCTGACTTCTTTTCTGATACTATCATCAAAAGTATC
ATTTCAGACATGTTTTTTGCAGGGCCAGAAACATCAACAATCACTTTAGAATGGGCAATGTCACTTCTTCTAAAT
CATCCAGAGGTATTGGGAAAGTTGAGAGCAGAGATTGATGATCATGTTGGACATGGACGCCTTCTAGATGACTCG
GATCTTGGGAAGCTTCCCTATCTCCGTTGCATCATCAATGAGACCCTCAGATTATATCCTCCAACACCACTTCTA
TTACCACACTGTTCATCTGAAGATTGCATTGTGGGGGGATATGAAATACCACAAGGTACAATCCTGTGGGTGAAT
GCTTGGGCCATGCATAGAGATCCCAAGTTGTGGGAGGAGCCAACCAAGTTCAAGCCTGAGAGATTTGAAGGCATG
GAAGGGAGAGAAAGGTATAAATTTATGCCATTTGGAATTGGGAGAAGAGCTTGTCCAGGTGCTAGTATGGCCATC
CGGACAGTTTCATTGGCATTGGGTGCACTTATTCAATGTTTTGAATGGGAAAACGTTGGGCCGGATAAAAGGGAG
ATGAGCCAGGGTCGACTTACTTTGCCCAAGGCCGAGTCTTTGGAGGCTGTGTCTATTCCACGCCCCAGTGCAGTG
AAAGTCCTCTCCCAGCTTGAAGGCACTTGTTTCCGTTAG
Coding nucleotide sequence of camptothecin hydroxylase Ca32229/CPT11H from
Camptotheca acuminata
SEQ ID NO: 2
ATGGAGAACTTGTACTACTGCCTTGCTCTCCTACTATCAATTCTTTTCATATTCAGACATTTCTTCCATCATAGT
TCAAAGTTACCACCAAGTCCATTTGCCTTTCCTATCATCGGCCATCTCCATCTCATCAGGAATTCTTTCCACCAA
GTACTAGAGTGCTTGGCATCTCAATATGGTCCAATTTTATTCCTCAAATTTGGCATCCGCTCTATTCTTGTTGTG
TCATCACCATCCGTTGTTGAGGAATGTTTTATTAAGAATGATATTATATTTGCAAACCGTCCTCGGAATATGCTT
TCAGATATCTCTAGTTATAATTATAGTACGATCGTAGGGGCTCCATATGGTCATTACTGGCGGAGCCTCCGCCGC
CTTGCTAGTGTTGAATTCTTCTCATTGAATAGCCTCCAGAAGTCTTCTAACATCCGTGAAGAGGAAATTCATAAC
CTTCTCTATCACCTCTTCAAATTCTCAAAAAGTGGAACTCAAAAAGTCCAGTTGAAATATTGGTTCTCTCTATTG
ACATTCAATATAATAACGAGGCTGGTAGCTGGGAAGCGGTGTGTTAGAGATGCGGTTGCAGGCATGGATTTGGGG
AAACAAATTCTTGAAGAACTCAAGGGGAAGTTCGTTTCGATCATGCCATTGAATATGTGTGATTTCTTTCCAATT
TTGAGGTGGTTTGGTTACAAAGGGCTGGAGAAAAATCTGATTACGTTGCACAAGGAGAGAGATGAATTCTTGCAG
GACTTGATAAATGAGGTTAGACGAAAGAGAACATGTTCTGCCAATATCAATATTGTAACAAACAAAGCAAAGACA
ACATTGATTGGAACTCTCTTGTCCTTCCAAGAATCAGAACCTGACTTCTTTTCTGATACTATCATCAAAAGTATC
ATTTCAGACATGTTTTTTGCAGGATCAGAAACATCAGCAATCACTCTAGAATGGGCAATGTCACTTCTTCTAAAT
CATCCAGAGGTATTGGGAAAGTTGAGAGCAGAGATTGATGATCATGTTGGACATGGACGCCTTCTAGATGACTCG
GATCTTGTGAAGCTTCCCTATCTTCGTTGCATCATCAATGAAACCCTCAGATTATATCCTCCAACACCACTTCTA
TTACCTCACTGTTCATCTGTAGATTGCACTGTGGGGGGATATGAAATACCACAAGGTACAATCCTGTGGGTGAAT
GCTTGGGCCATGCATAGAGATCCCAAGTTATGGGAGGAGCCAACCAAGTTCAAGCCTGAGAGATTTGAAGGCATG
GAAGGGAGAGAAAGGTACAAATTTATTCCATTTGGAATTGGGAGAAGAGCTTGTCCAGGTGCTAGTATGGGCATC
CGGACAGTTTCATTGGCTTTGGGCGCACTTATTCAGTGTTTTCAATGGGAAAACGTTGGGCAGGATAAAAGGGAG
ATGAGTCCGGTTCGACTTACGTTGCCCAAGGCCGAGTCTTTGGAGGCTATGTGTATTCCACGCCCCAGTGCAATG
AAAGTCCTCTCCCAGCTTGAAGACACTTGTTTCAGTTAG
Amino acid sequence of camptothecin hydroxylase Ca32236/CPT10H from
Camptotheca acuminata
SEQ ID NO. 3
MENLYYCLALLLSILFIFRHFFRHSSKLPPSPFALPIIGHLHLIRNSLHQVLECLASQYGPILFLKFGTRSILVV
SSPSAVEECLIKNDIIFANRPRSMILDLSSFNYSIFSWAPYGHYWRSLRRLAVVELFTSRSLQTSSNIRKEEIHN
LLCHLFKFSKSGTQKLQLKYWFSLLTFNIITRLVAGKRCVRDAVAGTDSGKQILEDLEGKFTSKMPFNMCDFFPI
LRWFGYKGLEKILITLHKERDEFMQGLIDEVRRKRTCSANINSVINRAKTTLIEALLSLQESEPDFFSDTIIKSI
ISDMFFAGPETSTITLEWAMSLLLNHPEVLGKLRAEIDDHVGHGRLLDDSDLGKLPYLRCIINETLRLYPPTPLL
LPHCSSEDCIVGGYEIPQGTILWVNAWAMHRDPKLWEEPTKFKPERFEGMEGRERYKFMPFGIGRRACPGASMAI
RTVSLALGALIQCFEWENVGPDKREMSQGRLTLPKAESLEAVSIPRPSAVKVLSQLEGTCF
Amino acid sequence of camptothecin hydroxylase Ca32229/CPT11H from
Camptotheca acuminata
SEQ ID NO: 4
MENLYYCLALLLSILFIFRHFFHHSSKLPPSPFAFPIIGHLHLIRNSFHQVLECLASQYGPILFLKFGIRSILVV
SSPSVVEECFIKNDIIFANRPRNMLSDISSYNYSTIVGAPYGHYWRSLRRLASVEFFSLNSLQKSSNIREEEIHN
LLYHLFKFSKSGTQKVQLKYWFSLLTFNIITRLVAGKRCVRDAVAGMDLGKQILEELKGKFVSIMPLNMCDFFPI
LRWFGYKGLEKNLITLHKERDEFLQDLINEVRRKRTCSANINIVTNKAKTTLIGTLLSFQESEPDFFSDTIIKSI
ISDMFFAGSETSAITLEWAMSLLLNHPEVLGKLRAEIDDHVGHGRLLDDSDLVKLPYLRCIINETLRLYPPTPLL
LPHCSSVDCTVGGYEIPQGTILWVNAWAMHRDPKLWEEPTKFKPERFEGMEGRERYKFIPFGIGRRACPGASMGI
RTVSLALGALIQCFQWENVGQDKREMSPVRLTLPKAESLEAMCIPRPSAMKVLSQLEDTCFS
Coding nucleotide sequence of putative CPT hydroxylase CPT10H ortholog 1
from Ophiorrhiza pumila
SEQ ID NO: 5
ATGGAGAATCTCTACTATTACTTAGTGTCAATCTTCTTGTGTGGTGTTTTCCTGATTCTATCCAAACAATTGTTG
TTCAACAAGAACAAGAAGTTACCTCCTAGTCCTCGTGTTCTTCCAATAATTGGCCATCTCCATCTCATCAAGAAC
GAATTCTATGAAGATTTTACTTCATTATCATCTACATACGGTCCAGTTTTCTTTCTCCGATTTGGCTGCCGGTCC
TATGTTGTTGTGTCTTCTCCATCTGCTGTTGGAGAGTGTTTCACAAAGAATGATATTATACTTGCAAACCGTCCT
AAGACCATGGCTGGGGACAGGTTGACCTATAACTATGGATCATTTGGAGTGGCTCCTTATGGGGATATATGGAGG
GTTCTTCGTCGCCTCACTGTTGTTGAATCTTTATCTTTCAACAGCCTCCAAAAGTCCTCAAATATCAGGGAAGAA
GAAATTCAGATGATTGTTCGTTCACTCTATCGAGTCTCAAAGAATGGAAGCCAACGAGTTGATTTGAACTATTGG
ATTTCAGTTTTTACACTCAATGTTATTATGAGGATGGTTACTGGAAGATGCTCAATTAGAGAGGAAGATGCTGGA
GACGAGTTGGGGAAGCAAATAGTTAAAGAATTCAAAGACAACTTTGCTACAGCCCTTTCAATGAGCTTGTGCGAC
TTCTTCCCGATATTAAGGTGGTTTGGTTACAAAGGGCTGGAAAAGAGAATGATCATTTTGCACAAGAAGAGAGAT
GCATTCCTTCAGGGTTTAGTAGATGAACTTCGATCAAATAAATCTAATTTTTCTCCTTCCGGCACTGGAATGAAC
GAAGAGAAGAAGAAGGCATTAATTCAATCCCTCCTTTCTCATCAGGAACTAGAACCTGATTTTCTCAAAGATGAC
TCTATAAAGAGTATTGCATTGTCCATCTTTCTAGCAGGAAGAGAAACGTCATCCATGACCATTGAATGGGCTATG
TCACTCTTACTGAATCACCAGGAAGCAATGCAGAAGTTAAGGACTGAAATCGACAACAACGTAGGACACAAAAGA
TTGTTGGATGAATCGGATATTCCAAAGCTTCCTTATCTGCGTTGTGTAGTGGATGAGACGATGAGACTGTATCCT
GCAGCACCACTGCTTCTTCCTCATTATGCGTCTGAAAATTGTAGAGTTTGTGACTATGACATTCCAAAAGGTACG
ACTGTTTTAACTAATGCTTGGGCCATACACAGGGATCCAAAACTCTGGGATATGCCTGAAAAGTTCATGCCAGAG
AGATTTGAGGCTAAACAAATAGGGGGAAAAGAAGAGTTCAATTTCAAGTTTCTACCATTTGGGATAGGGAGGAGA
GCATGTCCCGGAGCCAATTTGGCCATTCGGAACGTTTCTTTGGCATTGGGTGCATTGTTACAGTGCTTTTATTGG
GAAAAAGTTGGAGAGAAGGAAGGCGATATGGACAGTAAGAACGATGATAGAGTCACTTTGCAGAAGGCCAAACCC
TTGGAGGCCATTTGTTTTCCACGCCAAGAATCAATCCAACTTCTCTCGCAACTCTGA
Coding nucleotide sequence of putative camptothecin hydroxylase CPT10H
ortholog 2 from Ophiorrhiza pumila
SEQ ID NO: 6
ATGTCAACCCGAGTTCTTTGGGATAAGATTCCTATCAGACTAAGAGTTTTAATCCTACTGCAACTCTACCAGACT
TCATCAGTTTTCTTTCTCCGATTTGGCTGCCGGTCCTATGTTGTTGTGTCTTCTCCATCTGCTGTTGGAGAGTGT
TTCACAAAGAATGATATTATACTTGCAAACCGTCCTAAGACCATGGCTGGGGACAGGTTGACCTATAACTATGGA
TCATTTGGAGTGGCTCCGTATGGGGATATATGGAGGGTTCTTCGTCGCCTCACTGTTGTTGAATCTTTATCTTTC
AACAGCCTCCAAAAGTCCTCTAATATCAGGGAAGAAGAAATTCAGATGATTGTTCGTTCACTCTATCGAGTCTCA
AAGAATGGAAGCCAACGAGTTGATTTGAACTATTGGATTTCAGTTTTTACACTCAATGTTATTATGAGGATGGTT
ACTGGAAGATGCTCAATTAGAGAGGAAGATGCTGGAGACGAGTTGGGGAAGCAAATAGTTAAAGAATTCAAAGAC
AACTTTGCTACAGCCCTTTCAATGAGCTTGTGCGACTTCTTCCCGATATTAAGGTGGTTTGGTTACAAAGGGCTG
GAAAAGAGAATGATCATTTTGCACAAGAAGAGAGATGCATTCCTTCAGGGTTTAGTAGATGAACTTCGATCAAAT
AAATCTAATTTTTCTCCTTCCGGCACTGGAATGAACGAAGAGAAGAAGAAGGCATTAATTCAATCCCTCCTTTCT
CATCAGGAACTAGAACCTGATTTTCTCAAAGATGACTCTATAAAGAGTATTGCATTGTCCATCTTTCTAGCAGGA
AGAGAAACGTCATCCATGACCATTGAATGGGCTATGTCACTCTTACTGAATCACCAGGAAGCAATGCAGAAGTTA
AGGACTGAAATCGACAACAACGTAGGACACAAAAGATTGTTGGATGAATCGGATATTCCAAAGCTTCCTTATCTG
CGTTGTGTAGTGGATGAGACGATGAGACTGTATCCTGCAGCACCACTGCTTCTTCCTCATTATGCGTCTGAAAAT
TGTAGAGTTTGTGACTATGACATTCCAAAAGGTACGACTGTTTTAACTAATGCTTGGGCCATACACAGGGATCCA
AAACTCTGGGATATGCCTGAAAAGTTCATGCCAGAGAGATTTGAGGCTAAACAAATAGGGGGAAAAGAAGAGTTC
AATTTCAAGTTTCTACCATTTGGGATAGGGAGGAGAGCATGTCCCGGAGCCAATTTGGCCATTCGGAACGTTTCT
TTGGCATTGGGTGCATTGTTACAGTGCTTTTATTGGGAAAAAGTTGGAGAGAAGGAAGGCGATATGGACAGTAAG
AACGATGATAGAGTCACTTTGCAGAAGGCCAAACCCTTGGAGGCCATTTGTTTTCCACGCCAAGAATCAATCCAA
CTTCTCTCGCAACTCTGA
Coding nucleotide sequence of putative camptothecin hydroxylase CPT10H
ortholog 3 from Ophiorrhiza pumila
SEQ ID NO: 7
ATGGAGAATCTCTACTACTACTTAGTGTCAATCTTCTTGTGTGGTTTTTTCCTGATCCTATCCAAACAATTGTTT
TTCAACAAGAACAAGAAGTTACCTCCTAGTCCTCGTGCTCTTCCAATAATTGGCCATCTCCATCTCATCAAGAAC
GAACTCTATGAAGATTTTACTTCATTATCATCTACATACGGTCCAGTTTTCTTTCTCCGATTTGGCTGCCGGTCC
TATGTTGTTGTGTCTTCTCCATCTGCTGTTGAAGAGTGTTTCACAAAGAATGATATTATACTTGCAAACCGTGAT
AAGACCATGGCTGGGGACAGGTTGACCTATAACTATGGAACATTTGCAATGGCTCCTTATGGGGATATATGGAGG
GTTCTTCGTCGCCTCACTGTTGTTGAATCTTTATCTTTCAACAGACTCCAAAAGTCCTCAAATATCAGGGAAGAA
GAAATTCAGATGATTGTTCGTTCACTCTTTCGAGTCTCAAAGAATGGAAGCCAACGAGTTGATTTGAACTATTGG
ATTTCAGTTTTTACACTCAATGTTATTATGAGGATGGTTACTGGAAGATGCTCAATTAGAGAGGAAGATGCTGGA
GACGAGTTGGGGAAGCAAATAGTTAAAGAATTCAAAGACAACTTTGCTACAGGCCTTTCAATGAACTTGTGCGAC
TTCTTCCCGATATTAAGGTGGTTTGGTTACAAAGGGCTGGAAAAGAGAATGATCATTTTGCACAAGAAGAGAGAT
GCATTCCTTCAGGGTTTAGTAGATGAACTTCGATCAAATAAATCTAATTTTTCTCCTTCCGGCACTGGAATGAAC
GAAGAGAAGAAGAAGGCATTAATTGAATCCCTCCTTTCTCATCAGGAACTAGAACCTGATTTTCTCAAAGATGAC
TCTATAAAGAGTATTGCATTGTCCATCTTTATAGCAGGAAGAGAAACATCATCCATGACCATTGAATGGGCTATG
TCACTCTTACTGAATCACCCGGAAGCAATGCACAAGTTAAGGACTGAAATCGACAACAACGTAGGACACAAAAGA
TTGTTGGATGAATCGGATATTCCAAAGCTTCCTTATCTGCGTTGTGTCGTGGATGAGACATTGAGACTGTATCCT
CCAGCACCACTGCTTCTACCTCATTATGCATCTGAAAATTGTAGAGTTTGGGACTATGACATTCCAAAAGGTACG
ACTGTTTTAGCTAATGCTTGGGCCATACACAGGGATCCAAAACTCTGGGATATGCCTGAAAAGTTCATGCCAGAG
AGATTTGAGGCTAAACAATTAGGGGAAAAAGAAGAGTTCAATTTCAAGTTTCTACCATTTGGGATAGGGAGGAGA
GCATGTCCCGGAGCCAATTTGGGCATTCGGAACGTTTCTTTGGCATTGGGTGCATTGTTACAGTGCTTTTATTGG
GAAAAAGTTGGAGAGAAGGAAGGCGATATGGACATGATAGTGGATAGAGCCATAGAGTTCTATTTTGCCATGGAG
AATCTCTACTACTACTTAGTCTCAATCTTTTTGTGTTGCTTTTTCGTGATCCTATTCCTATCCAAACAATTGCTG
TTCAACAAGAACAAGAAGTTGCCACCCAGTCCTCCTGCTCTTCCAATAATTGGCCATCTCCATCTCATCAAGAAC
GAACTCTATCGAGATTTAACTTCATTATCATCTACATACGGTCCAGTTTTCTTTCTCAAATTTGGTTGCCGGTCC
TATGTTGTTGTGTCTTCTCCATCTGCCGTTGAAGAGTGCTTCACAAAGAATGATAATATACTTGCAAACCGTCCT
AACACCATGGCTTCGGACATTTTTACCTATAACTACTCAACAATTGGATCGGCTCCTTATGGGAATTTATGGAGG
GTTCTTCGTCGCCTCACTGTTGCTGAATCTTTATCATCCAACAGCCTTCAGAAGTCCTCAAATATCAGGGAAGAA
GAAATTCAGATGATTGTTCGTTCACTCTTTCGAATCTCAAAGAATGGAAGCCAACGAGTTGATTTGAACTACTGG
ATTTCAGTTTTTACACTCAATATTATTACGAGGATGATTACTGGAAGATGCTCAATTAGAGAGGAGGATGCCGGA
GATGAGTTGGGGAAGCAAATAGCTAAAGAATTCAAAGATAGGTTTGCTTCAGGCACTGCAATGAACTTGTGCGAC
TTCTTTCCGATATTAAGGTGGTTTGGTTACAAAGGGTTGGAAAAGAAAATGATCAGTTTGTACAAGAAGAGAGAT
GCATTCCTTCAGGGTTTAGTAGATAAATTTCGATCAGATAAATCTAACAACGAAGAGAAGAAGAAGACCATAATT
GAATCTCTCCTCTCTCATCAGGAAGAACTAGAACTAAAGCCTGATTTTCTCTCAGATGGCTTAATAAAGAGTACT
GCGCTGTCCATCTTTATAGCAGGAAGAGAAACATCATCCCTGACCATTGAATGGGCTATGTCACTCTTACTGAAA
CACCCGAAAGCAATGCACAAGTTAAGGACTGAAATCGACAACAATGTAGGACACAAAAGATTGTTGGATGAATCG
GATATTCCAAAGCTTCCTTATCTGCGTTGTGTCGTGGATGAGACATTGAGACTGTATCCTCCAGCACCACTGCTT
CTACCTCATTATGCATCTGAAAATTGTAGAGTTTGGGACTATGACATTCCAAAAGGTACGACTGTTTTAGCTAAC
GCTTGGGCCATACACAGGGATCCAAAACTCTGGGATATGCCTGAAAAGTTCATGCCAGAGAGATTTGAGGCTAAA
CAATTAGGGGAAAAAGAAGAGTTCAATTTCAAGTTTCTACCATTTGGGATAGGGAGGAGAGCATGTCCCGGAGCC
AATTTGGGCATTCGGAACGTTTCTTTGGCATTGGGTGCATTGTTACAGTGCTTTTATTGGGACAAAGTTGGAGAA
AAGGAAGGTGATATGGACACTAACAACGACGATAAACTCACTTTGCATAAGGCCAAACCCTGCGAGGCCATGTGT
TTTCCACGCCAAGAATCAATCCAACTTCTCTCGCAACTCTGA
Amino acid sequence of putative camptothecin hydroxylase CPT10H
ortholog 1 from Ophiorrhiza pumila
SEQ ID NO: 8
MENLYYYLVSIFLCGVFLILSKQLLFNKNKKLPPSPRVLPIIGHLHLIKNEFYEDFTSLSSTYGPVFFLRFGCRS
YVVVSSPSAVGECFTKNDIILANRPKTMAGDRLTYNYGSFGVAPYGDIWRVLRRLTVVESLSFNSLQKSSNIREE
EIQMIVRSLYRVSKNGSQRVDLNYWISVFTLNVIMRMVTGRCSIREEDAGDELGKQIVKEFKDNFATALSMSLCD
FFPILRWFGYKGLEKRMIILHKKRDAFLQGLVDELRSNKSNFSPSGTGMNEEKKKALIQSLLSHQELEPDFLKDD
SIKSIALSIFLAGRETSSMTIEWAMSLLLNHQEAMQKLRTEIDNNVGHKRLLDESDIPKLPYLRCVVDETMRLYP
AAPLLLPHYASENCRVCDYDIPKGTTVLTNAWAIHRDPKLWDMPEKFMPERFEAKQIGGKEEFNEKFLPFGIGRR
ACPGANLAIRNVSLALGALLQCFYWEKVGEKEGDMDSKNDDRVTLQKAKPLEAICFPRQESIQLLSQL
Amino acid sequence of putative camptothecin hydroxylase CPT10H ortholog 2
from Ophiorrhiza pumila
SEQ ID NO: 9
MSTRVLWDKIPIRLRVLILLQLYQTSSVFFLRFGCRSYVVVSSPSAVGECFTKNDIILANRPKTMAGDRLTYNYG
SFGVAPYGDIWRVLRRLTVVESLSFNSLQKSSNIREEEIQMIVRSLYRVSKNGSQRVDLNYWISVFTLNVIMRMV
TGRCSIREEDAGDELGKQIVKEFKDNFATALSMSLCDFFPILRWFGYKGLEKRMIILHKKRDAFLQGLVDELRSN
KSNFSPSGTGMNEEKKKALIQSLLSHQELEPDFLKDDSIKSIALSIFLAGRETSSMTIEWAMSLLLNHQEAMQKL
RTEIDNNVGHKRLLDESDIPKLPYLRCVVDETMRLYPAAPLLLPHYASENCRVCDYDIPKGTTVLTNAWAIHRDP
KLWDMPEKFMPERFEAKQIGGKEEFNFKFLPFGIGRRACPGANLAIRNVSLALGALLQCFYWEKVGEKEGDMDSK
NDDRVTLQKAKPLEAICFPRQESIQLLSQL
Amino acid sequence of putative camptothecin hydroxylase CPT10H ortholog 3
from Ophiorrhiza pumila
SEQ ID NO: 10
MENLYYYLVSIFLCCFFVILFLSKQLLFNKNKKLPPSPPALPIIGHLHLIKNELYRDLTSLSSTYGPVFFLKFGC
RSYVVVSSPSAVEECFTKNDNILANRPNTMASDIFTYNYSTIGSAPYGNLWRVLRRLTVAESLSSNSLQKSSNIR
EEEIQMIVRSLFRISKNGSQRVDLNYWISVFTLNIITRMITGRCSIREEDAGDELGKQIAKEFKDRFASGTAMNL
CDFFPILRWFGYKGLEKKMISLYKKRDAFLQGLVDKFRSDKSNNEEKKKTIIESLLSHQEELELKPDFLSDGLIK
STALSIFIAGRETSSLTIEWAMSLLLKHPKAMHKLRTEIDNNVGHKRLLDESDIPKLPYLRCVVDETLRLYPPAP
LLLPHYASENCRVWDYDIPKGTTVLANAWAIHRDPKLWDMPEKFMPERFEAKQLGEKEEFNFKELPFGIGRRACP
GANLGIRNVSLALGALLQCFYWDKVGEKEGDMDTNNDDKLTLHKAKPCEAMCFPRQESIQLLSQL
Coding nucleotide sequence of putative camptothecin hydroxylase CPT11H
ortholog 1 from Ophiorrhiza pumila
SEQ ID NO: 11
ATGGAGAATCTCTACTACTACTTAGTGTCAATCTTCTTGTGTGGTTTTTTCCTGATCCTATCCAAACAATTGTTT
TTCAACAAGAACAAGAAGTTACCTCCTAGTCCTCGTGCTCTTCCAATAATTGGCCATCTCCATCTCATCAAGAAC
GAACTCTATGAAGATTTTACTTCATTATCATCTACATACGGTCCAGTTTTCTTTCTCCGATTTGGCTGCCGGTCC
TATGTTGTTGTGTCTTCTCCATCTGCTGTTGAAGAGTGTTTCACAAAGAATGATATTATACTTGCAAACCGTGAT
AAGACCATGGCTGGGGACAGGTTGACCTATAACTATGGAACATTTGCAATGGCTCCTTATGGGGATATATGGAGG
GTTCTTCGTCGCCTCACTGTTGTTGAATCTTTATCTTTCAACAGACTCCAAAAGTCCTCAAATATCAGGGAAGAA
GAAATTCAGATGATTGTTCGTTCACTCTTTCGAGTCTCAAAGAATGGAAGCCAACGAGTTGATTTGAACTATTGG
ATTTCAGTTTTTACACTCAATGTTATTATGAGGATGGTTACTGGAAGATGCTCAATTAGAGAGGAAGATGCTGGA
GACGAGTTGGGGAAGCAAATAGTTAAAGAATTCAAAGACAACTTTGCTACAGGCCTTTCAATGAACTTGTGCGAC
TTCTTCCCGATATTAAGGTGGTTTGGTTACAAAGGGCTGGAAAAGAGAATGATCATTTTGCACAAGAAGAGAGAT
GCATTCCTTCAGGGTTTAGTAGATGAACTTCGATCAAATAAATCTAATTTTTCTCCTTCCGGCACTGGAATGAAC
GAAGAGAAGAAGAAGGCATTAATTGAATCCCTCCTTTCTCATCAGGAACTAGAACCTGATTTTCTCAAAGATGAC
TCTATAAAGAGTATTGCATTGTCCATCTTTATAGCAGGAAGAGAAACATCATCCATGACCATTGAATGGGCTATG
TCACTCTTACTGAATCACCCGGAAGCAATGCACAAGTTAAGGACTGAAATCGACAACAACGTAGGACACAAAAGA
TTGTTGGATGAATCGGATATTCCAAAGCTTCCTTATCTGCGTTGTGTCGTGGATGAGACATTGAGACTGTATCCT
CCAGCACCACTGCTTCTACCTCATTATGCATCTGAAAATTGTAGAGTTTGGGACTATGACATTCCAAAAGGTACG
ACTGTTTTAGCTAATGCTTGGGCCATACACAGGGATCCAAAACTCTGGGATATGCCTGAAAAGTTCATGCCAGAG
AGATTTGAGGCTAAACAATTAGGGGAAAAAGAAGAGTTCAATTTCAAGTTTCTACCATTTGGGATAGGGAGGAGA
GCATGTCCCGGAGCCAATTTGGGCATTCGGAACGTTTCTTTGGCATTGGGTGCATTGTTACAGTGCTTTTATTGG
GAAAAAGTTGGAGAGAAGGAAGGCGATATGGACATGATAGTGGATAGAGCCATAGAGTTCTATTTTGCCATGGAG
AATCTCTACTACTACTTAGTCTCAATCTTTTTGTGTTGCTTTTTCGTGATCCTATTCCTATCCAAACAATTGCTG
TTCAACAAGAACAAGAAGTTGCCACCCAGTCCTCCTGCTCTTCCAATAATTGGCCATCTCCATCTCATCAAGAAC
GAACTCTATCGAGATTTAACTTCATTATCATCTACATACGGTCCAGTTTTCTTTCTCAAATTTGGTTGCCGGTCC
TATGTTGTTGTGTCTTCTCCATCTGCCGTTGAAGAGTGCTTCACAAAGAATGATAATATACTTGCAAACCGTCCT
AACACCATGGCTTCGGACATTTTTACCTATAACTACTCAACAATTGGATCGGCTCCTTATGGGAATTTATGGAGG
GTTCTTCGTCGCCTCACTGTTGCTGAATCTTTATCATCCAACAGCCTTCAGAAGTCCTCAAATATCAGGGAAGAA
GAAATTCAGATGATTGTTCGTTCACTCTTTCGAATCTCAAAGAATGGAAGCCAACGAGTTGATTTGAACTACTGG
ATTTCAGTTTTTACACTCAATATTATTACGAGGATGATTACTGGAAGATGCTCAATTAGAGAGGAGGATGCCGGA
GATGAGTTGGGGAAGCAAATAGCTAAAGAATTCAAAGATAGGTTTGCTTCAGGCACTGCAATGAACTTGTGCGAC
TTCTTTCCGATATTAAGGTGGTTTGGTTACAAAGGGTTGGAAAAGAAAATGATCAGTTTGTACAAGAAGAGAGAT
GCATTCCTTCAGGGTTTAGTAGATAAATTTCGATCAGATAAATCTAACAACGAAGAGAAGAAGAAGACCATAATT
GAATCTCTCCTCTCTCATCAGGAAGAACTAGAACTAAAGCCTGATTTTCTCTCAGATGGCTTAATAAAGAGTACT
GCGCTGTCCATCTTTATAGCAGGAAGAGAAACATCATCCCTGACCATTGAATGGGCTATGTCACTCTTACTGAAA
CACCCGAAAGCAATGCACAAGTTAAGGACTGAAATCGACAACAATGTAGGACACAAAAGATTGTTGGATGAATCG
GATATTCCAAAGCTTCCTTATCTGCGTTGTGTCGTGGATGAGACATTGAGACTGTATCCTCCAGCACCACTGCTT
CTACCTCATTATGCATCTGAAAATTGTAGAGTTTGGGACTATGACATTCCAAAAGGTACGACTGTTTTAGCTAAC
GCTTGGGCCATACACAGGGATCCAAAACTCTGGGATATGCCTGAAAAGTTCATGCCAGAGAGATTTGAGGCTAAA
CAATTAGGGGAAAAAGAAGAGTTCAATTTCAAGTTTCTACCATTTGGGATAGGGAGGAGAGCATGTCCCGGAGCC
AATTTGGGCATTCGGAACGTTTCTTTGGCATTGGGTGCATTGTTACAGTGCTTTTATTGGGACAAAGTTGGAGAA
AAGGAAGGTGATATGGACACTAACAACGACGATAAACTCACTTTGCATAAGGCCAAACCCTGCGAGGCCATGTGT
TTTCCACGCCAAGAATCAATCCAACTTCTCTCGCAACTCTGA
Coding nucleotide sequence of putative camptothecin hydroxylase CPT11H
ortholog 2 from Ophiorrhiza pumila
SEQ ID NO: 12
ATGTCAACCCGAGTTCTTTGGGATAAGATTCCTATCAGACTAAGAGTTTTAATCCTACTGCAACTCTACCAGACT
TCATCAGTTTTCTTTCTCCGATTTGGCTGCCGGTCCTATGTTGTTGTGTCTTCTCCATCTGCTGTTGGAGAGTGT
TTCACAAAGAATGATATTATACTTGCAAACCGTCCTAAGACCATGGCTGGGGACAGGTTGACCTATAACTATGGA
TCATTTGGAGTGGCTCCGTATGGGGATATATGGAGGGTTCTTCGTCGCCTCACTGTTGTTGAATCTTTATCTTTC
AACAGCCTCCAAAAGTCCTCTAATATCAGGGAAGAAGAAATTCAGATGATTGTTCGTTCACTCTATCGAGTCTCA
AAGAATGGAAGCCAACGAGTTGATTTGAACTATTGGATTTCAGTTTTTACACTCAATGTTATTATGAGGATGGTT
ACTGGAAGATGCTCAATTAGAGAGGAAGATGCTGGAGACGAGTTGGGGAAGCAAATAGTTAAAGAATTCAAAGAC
AACTTTGCTACAGCCCTTTCAATGAGCTTGTGCGACTTCTTCCCGATATTAAGGTGGTTTGGTTACAAAGGGCTG
GAAAAGAGAATGATCATTTTGCACAAGAAGAGAGATGCATTCCTTCAGGGTTTAGTAGATGAACTTCGATCAAAT
AAATCTAATTTTTCTCCTTCCGGCACTGGAATGAACGAAGAGAAGAAGAAGGCATTAATTCAATCCCTCCTTTCT
CATCAGGAACTAGAACCTGATTTTCTCAAAGATGACTCTATAAAGAGTATTGCATTGTCCATCTTTCTAGCAGGA
AGAGAAACGTCATCCATGACCATTGAATGGGCTATGTCACTCTTACTGAATCACCAGGAAGCAATGCAGAAGTTA
AGGACTGAAATCGACAACAACGTAGGACACAAAAGATTGTTGGATGAATCGGATATTCCAAAGCTTCCTTATCTG
CGTTGTGTAGTGGATGAGACGATGAGACTGTATCCTGCAGCACCACTGCTTCTTCCTCATTATGCGTCTGAAAAT
TGTAGAGTTTGTGACTATGACATTCCAAAAGGTACGACTGTTTTAACTAATGCTTGGGCCATACACAGGGATCCA
AAACTCTGGGATATGCCTGAAAAGTTCATGCCAGAGAGATTTGAGGCTAAACAAATAGGGGGAAAAGAAGAGTTC
AATTTCAAGTTTCTACCATTTGGGATAGGGAGGAGAGCATGTCCCGGAGCCAATTTGGCCATTCGGAACGTTTCT
TTGGCATTGGGTGCATTGTTACAGTGCTTTTATTGGGAAAAAGTTGGAGAGAAGGAAGGCGATATGGACAGTAAG
AACGATGATAGAGTCACTTTGCAGAAGGCCAAACCCTTGGAGGCCATTTGTTTTCCACGCCAAGAATCAATCCAA
CTTCTCTCGCAACTCTGA
Coding nucleotide sequence of putative camptothecin hydroxylase CPT11H
ortholog 3 from Ophiorrhiza pumila
SEQ ID NO: 13
ATGGAGAATCTCTACTATTACTTAGTGTCAATCTTCTTGTGTGGTGTTTTCCTGATTCTATCCAAACAATTGTTG
TTCAACAAGAACAAGAAGTTACCTCCTAGTCCTCGTGTTCTTCCAATAATTGGCCATCTCCATCTCATCAAGAAC
GAATTCTATGAAGATTTTACTTCATTATCATCTACATACGGTCCAGTTTTCTTTCTCCGATTTGGCTGCCGGTCC
TATGTTGTTGTGTCTTCTCCATCTGCTGTTGGAGAGTGTTTCACAAAGAATGATATTATACTTGCAAACCGTCCT
AAGACCATGGCTGGGGACAGGTTGACCTATAACTATGGATCATTTGGAGTGGCTCCTTATGGGGATATATGGAGG
GTTCTTCGTCGCCTCACTGTTGTTGAATCTTTATCTTTCAACAGCCTCCAAAAGTCCTCAAATATCAGGGAAGAA
GAAATTCAGATGATTGTTCGTTCACTCTATCGAGTCTCAAAGAATGGAAGCCAACGAGTTGATTTGAACTATTGG
ATTTCAGTTTTTACACTCAATGTTATTATGAGGATGGTTACTGGAAGATGCTCAATTAGAGAGGAAGATGCTGGA
GACGAGTTGGGGAAGCAAATAGTTAAAGAATTCAAAGACAACTTTGCTACAGCCCTTTCAATGAGCTTGTGCGAC
TTCTTCCCGATATTAAGGTGGTTTGGTTACAAAGGGCTGGAAAAGAGAATGATCATTTTGCACAAGAAGAGAGAT
GCATTCCTTCAGGGTTTAGTAGATGAACTTCGATCAAATAAATCTAATTTTTCTCCTTCCGGCACTGGAATGAAC
GAAGAGAAGAAGAAGGCATTAATTCAATCCCTCCTTTCTCATCAGGAACTAGAACCTGATTTTCTCAAAGATGAC
TCTATAAAGAGTATTGCATTGTCCATCTTTCTAGCAGGAAGAGAAACGTCATCCATGACCATTGAATGGGCTATG
TCACTCTTACTGAATCACCAGGAAGCAATGCAGAAGTTAAGGACTGAAATCGACAACAACGTAGGACACAAAAGA
TTGTTGGATGAATCGGATATTCCAAAGCTTCCTTATCTGCGTTGTGTAGTGGATGAGACGATGAGACTGTATCCT
GCAGCACCACTGCTTCTTCCTCATTATGCGTCTGAAAATTGTAGAGTTTGTGACTATGACATTCCAAAAGGTACG
ACTGTTTTAACTAATGCTTGGGCCATACACAGGGATCCAAAACTCTGGGATATGCCTGAAAAGTTCATGCCAGAG
AGATTTGAGGCTAAACAAATAGGGGGAAAAGAAGAGTTCAATTTCAAGTTTCTACCATTTGGGATAGGGAGGAGA
GCATGTCCCGGAGCCAATTTGGCCATTCGGAACGTTTCTTTGGCATTGGGTGCATTGTTACAGTGCTTTTATTGG
GAAAAAGTTGGAGAGAAGGAAGGCGATATGGACAGTAAGAACGATGATAGAGTCACTTTGCAGAAGGCCAAACCC
TTGGAGGCCATTTGTTTTCCACGCCAAGAATCAATCCAACTTCTCTCGCAACTCTGA
Amino acid sequence of putative camptothecin hydroxylase CPT11H ortholog 1
from Ophiorrhiza pumila
SEQ ID NO: 14
MENLYYYLVSIFLCCFFVILFLSKQLLFNKNKKLPPSPPALPIIGHLHLIKNELYRDLTSLSSTYGPVFFLKFGC
RSYVVVSSPSAVEECFTKNDNILANRPNTMASDIFTYNYSTIGSAPYGNLWRVLRRLTVAESLSSNSLQKSSNIR
EEEIQMIVRSLFRISKNGSQRVDLNYWISVFTLNIITRMITGRCSIREEDAGDELGKQIAKEFKDRFASGTAMNL
CDFFPILRWFGYKGLEKKMISLYKKRDAFLQGLVDKFRSDKSNNEEKKKTIIESLLSHQEELELKPDFLSDGLIK
STALSIFIAGRETSSLTIEWAMSLLLKHPKAMHKLRTEIDNNVGHKRLLDESDIPKLPYLRCVVDETLRLYPPAP
LLLPHYASENCRVWDYDIPKGTTVLANAWAIHRDPKLWDMPEKFMPERFEAKQLGEKEEFNFKELPFGIGRRACP
GANLGIRNVSLALGALLQCFYWDKVGEKEGDMDTNNDDKLTLHKAKPCEAMCFPRQESIQLLSQL
Amino acid sequence of putative camptothecin hydroxylase CPT11H ortholog 2
from Ophiorrhiza pumila
SEQ ID NO: 15
MSTRVLWDKIPIRLRVLILLQLYQTSSVFFLRFGCRSYVVVSSPSAVGECFTKNDIILANRPKTMAGDRLTYNYG
SFGVAPYGDIWRVLRRLTVVESLSENSLQKSSNIREEEIQMIVRSLYRVSKNGSQRVDLNYWISVFTLNVIMRMV
TGRCSIREEDAGDELGKQIVKEFKDNFATALSMSLCDFFPILRWFGYKGLEKRMIILHKKRDAFLQGLVDELRSN
KSNFSPSGTGMNEEKKKALIQSLLSHQELEPDFLKDDSIKSIALSIFLAGRETSSMTIEWAMSLLLNHQEAMQKL
RTEIDNNVGHKRLLDESDIPKLPYLRCVVDETMRLYPAAPLLLPHYASENCRVCDYDIPKGTTVLTNAWAIHRDP
KLWDMPEKFMPERFEAKQIGGKEEFNFKFLPFGIGRRACPGANLAIRNVSLALGALLQCFYWEKVGEKEGDMDSK
NDDRVTLQKAKPLEAICFPRQESIQLLSQL
Amino acid sequence of putative camptothecin hydroxylase CPT11H ortholog 3
from Ophiorrhiza pumila
SEQ ID NO: 16
MENLYYYLVSIFLCGVFLILSKQLLFNKNKKLPPSPRVLPIIGHLHLIKNEFYEDFTSLSSTYGPVFFLRFGCRS
YVVVSSPSAVGECFTKNDIILANRPKTMAGDRLTYNYGSFGVAPYGDIWRVLRRLTVVESLSFNSLQKSSNIREE
EIQMIVRSLYRVSKNGSQRVDLNYWISVFTLNVIMRMVTGRCSIREEDAGDELGKQIVKEFKDNFATALSMSLCD
FFPILRWFGYKGLEKRMIILHKKRDAFLQGLVDELRSNKSNFSPSGTGMNEEKKKALIQSLLSHQELEPDFLKDD
SIKSIALSIFLAGRETSSMTIEWAMSLLLNHQEAMQKLRTEIDNNVGHKRLLDESDIPKLPYLRCVVDETMRLYP
AAPLLLPHYASENCRVCDYDIPKGTTVLTNAWAIHRDPKLWDMPEKFMPERFEAKQIGGKEEFNFKFLPFGIGRR
ACPGANLAIRNVSLALGALLQCFYWEKVGEKEGDMDSKNDDRVTLQKAKPLEAICFPRQESIQLLSQL
Coding nucleotide sequence of putative camptothecin hydroxylase CPT10H
ortholog from Nothapodytes nimmoniana
SEQ ID NO: 17
ATGGAGATGCTTTACTTCTACCTTATTTTTCTGGTCTCAGTTCTCCTGATATTCAAACACATCTTCCATTTTAAC
AAAAGTAAATTACCACCAAGTCCTCCATATATTCCGATAATTGGCCACCTCTACCTCATAAAGGGTAGTATCCAC
CAAGCACTTCAGTCTCTGTCATCAAAATATGGTCCAATTCTATTCCTCCGGCTCGGCGTCCGGTCCATGTTGGTT
GTCTCTTCTCCCTCTGCCGTGGAAGAATGCTTCACCAAGAACGACATCATATTTGCAAACCGGCCCCGAACCTTG
GCCGGCGACCTGTTGACTTACAACTACAGAGCTTTCGTGTGGACTCCGTACGGACATATTTGGCGGAGCCTCCGC
CGTCTCTCGGTGGTTGAACTCTTCTCTTCAACCAGCGTCCACAGGTCTTCAGCAGTTCGTGAAGATGAAATCCGA
ACCCTCGTTCGACATCTCTATAAAGTATCAAAGAGTGGGAATCCAAAGGTGGAATTCAAGTACTGGTTCTCAATT
TGTTTGTTCAATACCATAACGAGGATTGTCGCCGGGAGACAGGTTGTACCGGAGGAAGACGCAGGCGGGGAGGCC
GGGCGGCGAATTATGGCAGACCTTAGAGAGAGATTCTTTACGAACGTCGGAATGAATATGTGCGATTTCCTTCCA
ATTCTGAGGTGGTTTGGTTACAAAGGGCTGGAAAAAAAATTGATGGTAGCGTTCAAAAGGAGGGACGAGTTCTTG
CAGGGCCTACTAGATGAGTTTCGATTAAAGAAAATGAATTCCTCATCTCAGAAACATGTGAAAGATGGAAAAGAG
AAAGGTCCGTTGATAGAAACTCTGTTGTCCCTTCGTGAATCAGAGCCTGAGTTCTACACCGTTGATGTCATCAAA
AGTTTAATGCTGGTAATGTTTGTGGCTGGAACAGAGACAACTGCAACTACTGTAGAGTGGGCAATGTCACTTCTT
CTAACACACCCTGAAACACTTGACAAGCTAAGAACAGAGATTGACAACAATGTCAGGGAAGAACGACTACTAACC
GACATGGATCTTTCTAAACTTCCTTATCTCCGTTGTGTTATCAACGAAGCCCTCAGATTGTACCCCCCAGTGCCA
CTTCTATTACCACATTTCTCATCTAAAGATTGTACAATTGGAGGGCATGTGATACCCGAAGGTACAATCCTAGTT
GTTAATTCTTGGGCATTGCAAAGGGATCCCAACGTTTGGGAGGAGCCACACAAGTTCAAGCCAGAGAGATTTGAG
ATGGAGGAGGAAAAAGAAGGGTTTGGTTATAAATTCGTTCCGTTTGGGGTAGGGAGGAGGGCATGCCCTGGAGTC
AATATGGGCATGAGGGCAGCTTTGTTGGCACTTGGTACACTGATTCAATGTTTTGAGTGGGAAAAGGTTGGCCAA
TTTGAGATGGAAATGAGGTACAATAATGGAGTAACTTTGCAGAAGGCTAAACCCTTTGAAGCTAATTGCAAACCA
AGACAAAATTTTGTTCAACTCCTTGGTCAGCTTTGA
Amino acid sequence of putative camptothecin hydroxylase CPT10H ortholog
from Nothapodytes nimmoniana
SEQ ID NO: 18
MEMLYFYLIFLVSVLLIFKHIFHFNKSKLPPSPPYIPIIGHLYLIKGSIHQALQSLSSKYGPILFLRLGVRSMLV
VSSPSAVEECFTKNDIIFANRPRTLAGDLLTYNYRAFVWTPYGHIWRSLRRLSVVELFSSTSVHRSSAVREDEIR
TLVRHLYKVSKSGNPKVEFKYWFSICLFNTITRIVAGRQVVPEEDAGGEAGRRIMADLRERFFTNVGMNMCDFLP
ILRWFGYKGLEKKLMVAFKRRDEFLQGLLDEFRLKKMNSSSQKHVKDGKEKGPLIETLLSLRESEPEFYTVDVIK
SLMLVMFVAGTETTATTVEWAMSLLLTHPETLDKLRTEIDNNVREERLLTDMDLSKLPYLRCVINEALRLYPPVP
LLLPHFSSKDCTIGGHVIPEGTILVVNSWALQRDPNVWEEPHKFKPERFEMEEEKEGFGYKFVPFGVGRRACPGV
NMGMRAALLALGTLIQCFEWEKVGQFEMEMRYNNGVTLQKAKPFEANCKPRQNFVQLLGQL
Coding nucleotide sequence of putative camptothecin hydroxylase CPT11H
ortholog from Nothapodytes nimmoniana
SEQ ID NO: 19
ATGGAGATGCTTTACTTCTACCTTATTTTTCTGGTCTCAGTTCTCCTGATATTCAAACACATCTTCCATTTTAAC
AAAAGTAAATTACCACCAAGTCCTCCATATATTCCGATAATTGGCCACCTCTACCTCATAAAGGGTAGTATCCAC
CAAGCACTTCAGTCTCTGTCATCAAAATATGGTCCAATTCTATTCCTCCGGCTCGGCGTCCGGTCCATGTTGGTT
GTCTCTTCTCCCTCTGCCGTGGAAGAATGCTTCACCAAGAACGACATCATATTTGCAAACCGGCCCCGAACCTTG
GCCGGCGACCTGTTGACTTACAACTACAGAGCTTTCGTGTGGACTCCGTACGGACATATTTGGCGGAGCCTCCGC
CGTCTCTCGGTGGTTGAACTCTTCTCTTCAACCAGCGTCCACAGGTCTTCAGCAGTTCGTGAAGATGAAATCCGA
ACCCTCGTTCGACATCTCTATAAAGTATCAAAGAGTGGGAATCCAAAGGTGGAATTCAAGTACTGGTTCTCAATT
TGTTTGTTCAATACCATAACGAGGATTGTCGCCGGGAGACAGGTTGTACCGGAGGAAGACGCAGGCGGGGAGGCC
GGGCGGCGAATTATGGCAGACCTTAGAGAGAGATTCTTTACGAACGTCGGAATGAATATGTGCGATTTCCTTCCA
ATTCTGAGGTGGTTTGGTTACAAAGGGCTGGAAAAAAAATTGATGGTAGCGTTCAAAAGGAGGGACGAGTTCTTG
CAGGGCCTACTAGATGAGTTTCGATTAAAGAAAATGAATTCCTCATCTCAGAAACATGTGAAAGATGGAAAAGAG
AAAGGTCCGTTGATAGAAACTCTGTTGTCCCTTCGTGAATCAGAGCCTGAGTTCTACACCGTTGATGTCATCAAA
AGTTTAATGCTGGTAATGTTTGTGGCTGGAACAGAGACAACTGCAACTACTGTAGAGTGGGCAATGTCACTTCTT
CTAACACACCCTGAAACACTTGACAAGCTAAGAACAGAGATTGACAACAATGTCAGGGAAGAACGACTACTAACC
GACATGGATCTTTCTAAACTTCCTTATCTCCGTTGTGTTATCAACGAAGCCCTCAGATTGTACCCCCCAGTGCCA
CTTCTATTACCACATTTCTCATCTAAAGATTGTACAATTGGAGGGCATGTGATACCCGAAGGTACAATCCTAGTT
GTTAATTCTTGGGCATTGCAAAGGGATCCCAACGTTTGGGAGGAGCCACACAAGTTCAAGCCAGAGAGATTTGAG
ATGGAGGAGGAAAAAGAAGGGTTTGGTTATAAATTCGTTCCGTTTGGGGTAGGGAGGAGGGCATGCCCTGGAGTC
AATATGGGCATGAGGGCAGCTTTGTTGGCACTTGGTACACTGATTCAATGTTTTGAGTGGGAAAAGGTTGGCCAA
TTTGAGATGGAAATGAGGTACAATAATGGAGTAACTTTGCAGAAGGCTAAACCCTTTGAAGCTAATTGCAAACCA
AGACAAAATTTTGTTCAACTCCTTGGTCAGCTTTGA
Amino acid sequence of putative camptothecin hydroxylase CPT11H ortholog
from Nothapodytes nimmoniana
SEQ ID NO: 20
MEMLYFYLIFLVSVLLIFKHIFHFNKSKLPPSPPYIPIIGHLYLIKGSIHQALQSLSSKYGPILFLRLGVRSMLV
VSSPSAVEECFTKNDIIFANRPRTLAGDLLTYNYRAFVWTPYGHIWRSLRRLSVVELFSSTSVHRSSAVREDEIR
TLVRHLYKVSKSGNPKVEFKYWFSICLFNTITRIVAGRQVVPEEDAGGEAGRRIMADLRERFFTNVGMNMCDFLP
ILRWFGYKGLEKKLMVAFKRRDEFLQGLLDEFRLKKMNSSSQKHVKDGKEKGPLIETLLSLRESEPEFYTVDVIK
SLMLVMFVAGTETTATTVEWAMSLLLTHPETLDKLRTEIDNNVREERLLTDMDLSKLPYLRCVINEALRLYPPVP
LLLPHFSSKDCTIGGHVIPEGTILVVNSWALQRDPNVWEEPHKFKPERFEMEEEKEGFGYKFVPFGVGRRACPGV
NMGMRAALLALGTLIQCFEWEKVGQFEMEMRYNNGVTLQKAKPFEANCKPRQNEVQLLGQL
Coding nucleotide sequence of putative camptothecin hydroxylase Ca6009 from
Camptotheca acuminata
SEQ ID NO: 21
ATGGAGAACATATACTACTACCTTGCTCTCCTCTTGTCTGTTCTCTTCATGTTCAAACATTTCTTCCATCACAAT
CGGAAGTTACCACCAAGTCCGCTTGCGCTTCCAATTATTGGCCACCTCCACCTTATCAAGAAGTTGCTACACCAG
TCACTAGAGTGTCTTTCATCCCGATATGGTCCAATTTTATTTCTCCAATTTGGCTCCCGTTCCGTTGTTGCTTTA
TCTTCTCCATCTGCCGTTGAAGAATGCTTCACCAAAAATGACATAATATTTGCAAACCGGCCTCGAACAATGGCT
GGGGATCATTTCACTTACAATTATACTGCCTTTGTATGGGCTCCATATGGTCATCTCTGGCGGAGTCTCCGCCGT
CTGACTGTCATTGAGCTCTTCTCTTCAAACAGCCTTCAGAAGTCTTCTTTTGTTCGTGAGGGGGAAATTGGTAAT
CTTCTATGTCACCTGTTCAAATTCTCAAACAATGGAACTCAAAAAGTCGAGTTGAAGTATTGGTTCTCTCTTTTG
GCATTTAATATCATGATGAAGATGATTGCTGGAAAGCGATGTGTTAGAGATGAGGTTGCAGGCATGGAGGCAGGG
AAGCAAATTCTTGAAGATCTCAGGGGAAAGTTCGTTTCAACCACACCATTGAATATTTGTGATTTCTTTCCAATT
TTGAGGTGGCTTGGCTACAAAGGGCTGAAGAAGAGTATGATAAGGTTGCACAAGAAGAGAGATGAATTCTTGCAG
GGTTTGATAGATGAGTTTCGAATTAAAAGCAGTTCTTCTGCCAATACCAATGCTATAATGCACAGGGTACAAAAG
GTAACATTGATTGAGAAACTCTTGTCTCTGCAAGAAGCAGAACCTGACTTCTATTCGGATGACGTTATCAAAAGT
ATCATATTGGTAACTTTTGTGGCAGGTACCGAAACATCAGCAGTCACTATAGAATGGGCAATGTCACTTCTTCTA
AATAATCCACAGGCATTGGTGAAGGTGAAAGCAGAGATTTCCAGTCATGTCGGATTTGAGCGCTTGCTAAATGAC
TCTGATCTTCCCAAGCTACATTATCTCCGTTGTGTCATCAATGAGACGCTCAGATTATATCCTCCGGTGCCACTC
CTGTTACCACACTACTCATCGAAAGATTGCACTTTAGGGGGGTATGAAATTCCACAAGGTACAATTCTAACTGTG
AATGCTTGGGCAATGCATAGGGATCCCAAGGTGTGGGAAGATCCCACCAAGTTCAACCCTGAGAGATTTGAAGTT
GTTCAAGGGGAAAGAGAAGGGTTCAAATTTATTCCATTTGGAGTGGGGAGGAGAGCTTGTCCAGGTGCAGCTATG
GCCTTGCGGACAGTTTCATTAGCTTTGGGTGCACTGATTCAATGTTTTGAATGGGAAAAGGTTGGACAGGAGAAT
ATGGAGACGAGTCAGGGAGGACTGACTTTGCCCAAGGCTGGGTGTTTGGAGGCTGTGTGCATTCCACGCCAAGAT
TCGATTAAACTGCTATCCCAACTTGAAAGCCATTGTTCTGATTAA
Amino acid sequence of putative camptothecin hydroxylase Ca6009 from
Camptotheca acuminata
SEQ ID NO: 22
MENIYYYLALLLSVLFMFKHFFHHNRKLPPSPLALPIIGHLHLIKKLLHQSLECLSSRYGPILFLQFGSRSVVAL
SSPSAVEECFTKNDIIFANRPRTMAGDHFTYNYTAFVWAPYGHLWRSLRRLTVIELFSSNSLQKSSFVREGEIGN
LLCHLFKFSNNGTQKVELKYWFSLLAFNIMMKMIAGKRCVRDEVAGMEAGKQILEDLRGKFVSTTPLNICDFFPI
LRWLGYKGLKKSMIRLHKKRDEFLQGLIDEFRIKSSSSANTNAIMHRVQKVTLIEKLLSLQEAEPDFYSDDVIKS
IILVTFVAGTETSAVTIEWAMSLLLNNPQALVKVKAEISSHVGFERLLNDSDLPKLHYLRCVINETLRLYPPVPL
LLPHYSSKDCTLGGYEIPQGTILTVNAWAMHRDPKVWEDPTKFNPERFEVVQGEREGFKFIPFGVGRRACPGAAM
ALRTVSLALGALIQCFEWEKVGQENMETSQGGLTLPKAGCLEAVCIPRQDSIKLLSQLESHCSD
Coding nucleotide sequence of putative camptothecin hydroxylase Ca6007 from
Camptotheca acuminata
SEQ ID NO: 23
ATGGATATGGATATGGATACGGGGCTCGTCTTCTGCATAATAGTTATCATCTTCTCCATCCTTTTCATTTCCAAA
TTTCTATTGCCACAAAGGAAAAACTTTCCACCGAGTCCACTCGCTCTTCCCATACTCGGCCATCTCCACCTCCTC
AAGAATCCGGTGCACAGGGCGCTCCAGTCTCTGTCCAATCAGCACGGCCCAATCCTACTGCTGCGGTTCGGATCC
CGCCCTGTCCTTGTCGTCTCGTCTCCGTCGGCCGCCCAACAATGCTTCACCGCTGAAAACGACGTTATCTTCGCA
AACCGACCCAACACCCTCGGCGGCAAACACTTCGGCTACAACTACACTACTCTTGGGTGGTCCCCCTACGGCGAC
CGGTGGCGCGATCTCCGCCGTATCACCACCATCCAAATCTTCTCCTCCAAGAGTCTCCAGGATTCTGCCACGGTC
CGAAGAGAGGAGGTCCGGTTTATCACCCGCCAGCTGTTTCTGGGATCCGAAGGATCGACCCAGAAGGTGAACGTG
CAATATCTGGCCTTCCAGCTGACCTTCAACTTGACGATGAAGATGGTCGCTGGAAAAAGGTGTTCAAGGGCGAAG
GAGATATTCGCTCCGATGATGCGGATGAATAAATTAGATTTCTTACCCTTTTTGAAGTGGTTTGGTCTCAAAGGA
TCGGAGAATGGGTTGGTGAAGTTACAGAAGGCAAGAGATGCATTCTTGCAGGGCTTGATCGATGAGTATCGACCG
GAGAGGGAAGTGGACAAGAAGAAGACGATGATCGAGACTTTGTTGTCTTTTCAAGAAGAAGACCCTGAATTTTTC
ACGGAAAATACAGTCAAGGGCATCATGGTGCTACTATTTACAGCGGGAACAGATACTGTAGCTCGCACAATGGAA
TGGGCAATGTCACTTCTCCTGAATCACCCAGAAGTACTGCAAAAGGCCAGAAGCGAAATAGACAATCATGTAAAG
CCACATCGTCTGCTAGAGGACTCTGATCTTTCCAAACTACCTTATCTACGTTGCATCATCAACGAAACTCTTCGA
TTATTTCCTGTTGCACCACTTCTCGTACCTCATTTTTCATCAGAAGACTGCTTAGTAGAGAGATTCCATGTTCCA
CGAGGAACAATTTTGTTGGTCAATGCTTGGGCCATTCATAGGGATCCCAGTGTCTGGGAAGAGCCCACCAAGTTT
AAGCCAGAGAGGTTTGAAGGAATTGAAGGGGAACGAGAAGGGTTCAAGTTCATACCATTTGGGGTGGGGAGGAGG
GGATGTCCTGGTGCTGGCTTGGCTCTGCGTTTGCTTGGGTTGGCCTTGGGGACATTGATTCAGTGCTTTGAGTGG
GAAAGGGTTGGGTCTGAATTGGTGGACTTGACCGAGGGCAGTGGGATAACTTTGCTAAAGGTTAAGCCATTAGAG
GCCATGTATAGACCTCGCCGGTCCATGACCGCTCTCCTTTCTCAACTTTGA
Amino acid sequence of putative camptothecin hydroxylase Ca6007 from
Camptotheca acuminata
SEQ ID NO: 24
MDMDMDTGLVFCIIVIIFSILFISKFLLPQRKNFPPSPLALPILGHLHLLKNPVHRALQSLSNQHGPILLLRFGS
RPVLVVSSPSAAQQCFTAENDVIFANRPNTLGGKHFGYNYTTLGWSPYGDRWRDLRRITTIQIFSSKSLQDSATV
RREEVRFITRQLFLGSEGSTQKVNVQYLAFQLTFNLTMKMVAGKRCSRAKEIFAPMMRMNKLDFLPFLKWFGLKG
SENGLVKLQKARDAFLQGLIDEYRPEREVDKKKTMIETLLSFQEEDPEFFTENTVKGIMVLLFTAGTDTVARTME
WAMSLLLNHPEVLQKARSEIDNHVKPHRLLEDSDLSKLPYLRCIINETLRLFPVAPLLVPHFSSEDCLVERFHVP
RGTILLVNAWAIHRDPSVWEEPTKFKPERFEGIEGEREGFKFIPFGVGRRGCPGAGLALRLLGLALGTLIQCFEW
ERVGSELVDLTEGSGITLLKVKPLEAMYRPRRSMTALLSQL
Coding nucleotide sequence of putative camptothecin hydroxylase
Ca23831/CPT9H from Camptotheca acuminata
SEQ ID NO: 25
ATGGTCATTACTGGCGAAGCCTCCGCCGCCTTGCTATTGTTGAACTCTTCACATCGAACAGCCTTCAGAAGTCTT
CCAACATCCGTAAAGAGGAAATTCATAACCTTCTCTGTCACCTCTTCAAATTCTCAAAAAGTGGAGTTGAAATAT
TGGTTTTTTCGATTGACATTCAATATTATAACAAGGCTGGTAGCTGGGAAACAATGTGTTAGAGATGCACTTGCA
GGCACAGATTTGGGGAAACAAATTCTTGAAGACCTCGAGGGGAAGTTCGGTTCAAAAATGCCATTGAATATGTGT
GATTTCTTTCCAATTTTGAGGTGGTTTGGTTACAAAGGGCTGGAGAAAAGTCTGACAGTGTGGCACAAGGAGAGA
GATGAATTTATGCAAGGTTTGATAGATGAGGTTAGACGAAAGAGAACCTGTTCTGCCAATATCAATAATATAACA
AACAGAGCAAAGACAACATTGATTGAAGCTCTCTTGTCCCTCCAAGAATCACAACCTGACTTCTTTTCTGATACT
ATCATCAAAAGTATCATTTCAGACATGTTTTTTGCAGGGCCAGAAACATCAGCAATCACTCTAGAATGGGCAATG
TCACTTCTTCTAAATCATCCAGAGGTACTGCGAAAGTTAAGAGCAGGGATTGATGATCATGTTGGACATGGACGC
CTTCTAGATGACTCGGATCTTGTGAAGCTTCCCTATCTCCGTTGCATCATCAATGAGACCCTCAGATTATATCCT
CCAACACCACTTCTATTACCACACTGTTCATCTGAGGATTGCACTGTGGGGGGATATGAAATACCACAAGGTACA
ATCCTGTGGGTGAATGCTTGGGCCATGCATAGAGATCCCAAGTTATGGGAGGAGCCAACCAAGTTCAAGCCTGAG
AGATTTGAAGGCATGGAAGGGAGAGAAAGGAACAAATTTATTCCATTTGGAATTGGGAGAAGAGCTTGTCCAGGT
GCTAGTATGGGCATCCGGACAGTTTCATTGGCTTTGGGTGCACTTATTCAGTGTTTTGAATGGGAAAACGTTGGG
CAGAAGAAAATGGAGATGAGCCAAGGTCGACTTACTTTGCCCAAGGCCGAGTCTTTGGAGGCTACGTGTATTCCA
CGCCCTAGTGCAATGAAAGTCCTCTCCCAGCTTGAAGACACTTGTTTCAGTTAG
Amino acid sequence of putative camptothecin hydroxylase Ca23831/CPT9H from
Camptotheca acuminata
SEQ ID NO: 26
MVITGEASAALLLLNSSHRTAFRSLPTSVKRKFITFSVTSSNSQKVELKYWFFRLTFNIITRLVAGKQCVRDALA
GTDLGKQILEDLEGKFGSKMPLNMCDFFPILRWFGYKGLEKSLTVWHKERDEFMQGLIDEVRRKRTCSANINNIT
NRAKTTLIEALLSLQESQPDFFSDTIIKSIISDMFFAGPETSAITLEWAMSLLLNHPEVLRKLRAGIDDHVGHGR
LLDDSDLVKLPYLRCIINETLRLYPPTPLLLPHCSSEDCTVGGYEIPQGTILWVNAWAMHRDPKLWEEPTKFKPE
RFEGMEGRERNKFIPFGIGRRACPGASMGIRTVSLALGALIQCFEWENVGQKKMEMSQGRLTLPKAESLEATCIP
RPSAMKVLSQLEDTCES
Coding nucleotide sequence of putative camptothecin hydroxylase Ca23838
from Camptotheca acuminata
SEQ ID NO: 27
ATGGACACACTGTATACATCTCTTGCATTAATATTAGCCACATATTTCTTCATCAAACACTTCGTCATTCGCAAG
ATCCAAAACAAACCACCGAGTCCATTCCCATCGCTGCCCATTCTCGGCCACCTCCACCTCTTGAAGAAGCCCCTC
CACCGAACCTTGGCCCATATATCGGCCCGTTACGGCAGTATTATTCTCCTCCATTTCGGATCACGTCCAGTCGTC
GTAGTCTCATCTCCCTCAGCAGCGGAGGAATGCCTCACCAAGAACGACATCATCTTCGCCAATCGCCCTCGCCTC
CTCGCCGGAAAATACCTTGGCTACAACCATACCTCCCTCGCGTGGGCCCCCTATAGCGACCACTGGCGGAACCTC
CGCCGGATCGCGTCGCTCGAAATTCTGTCATCCCATAGGCTGCAGATGTTATCCGGCATACGCTCCGACGAGGTG
CGTTCGGTGGTTCGTAGACTTTCCCGGGCTTCCGCAGATGATCGGGTGGACATGAAAAAGGTATTCTTCGAGCTG
ATGCTTAACGTGATGATGAGAATGATTGCTGGAAAGAGGTATTACGGCGAGAACGTGGCGGAGGTAGAGCAGGGG
ACGCGGTTTCGCGAGATCGTGGTGGAGACATTCCTGCTTTCTGGAGCCACAAACATGGGGGACTTTTTGCCCATT
TTGAATTGGGTGGGAGTGACGGGATCGGAGAAGCGGTTGATGGCGTTGCAGAAGAAGAGAGATGCGTTTATGCAG
GAATTGATAGAAGAGCATAGAAGAGGAATGGGGATCGATAATGGCGATTCAGATGAGCAGGGAGAGAAAAAGAAG
ACGATGATTGCAGTTTTGTTATCCCTGCAAGAAACGGAACCTGATTATTACAAGGATGAAATTATCAGAGGCATC
ACGCTGGTTCTGTTAGCTGCAGGAACTGATACTTCAGCTGGGACCATGGAGTGGGCACTTTCACTTTTGTTGAAC
AATCCAGAAGTTCTAAAAAAGGCACAGATTGAAATTGATAATAAGGTTGGACAAAACCGTTTGGTCAATGAATCA
GACATAGCTGACCTCCCTTATCTCCGCTGCATCCTCAACGAGACCTTTCGGATGTTCCCGGTAGGCCCATTATTA
TTACCTCATGAATCATCAGAAGATTGCACGGTCGGAGGTTTCCACATCCCACGTGGCACTATGCTAATGATTAAT
TTGTGGGCCATACAAAATGACCCCAAGATTTGGGAGGACCCAAGAAAGTTCAAGCCAGAACGGTTTGAAGGACTG
GAAGGGGTAAGAGATGGTTTCAAATTGATGCCTTTTGGGTCAGGCAGGAGAGGGTGTCCTGGGGAGGGTCTGGCC
ATGCGAATGCTTGGCTTTACATTAGGGTCATTGATTCAGTGCTTTGATTGGGAAAGGGTTGGCAAGGACTTGGTG
GACTTGACTGAAGGGCCTGGGCTCACCATGCCCAAGGCTCAACCCTTGGTGGCTAAGTGCCGGCCACGTGCAACA
ATGTTGAACCTTCTGTCTCAAATTTGA
Amino acid sequence of putative camptothecin hydroxylase Ca23838 from
Camptotheca acuminata
SEQ ID NO: 28
MDTLYTSLALILATYFFIKHFVIRKIQNKPPSPFPSLPILGHLHLLKKPLHRTLAHISARYGSIILLHFGSRPVV
VVSSPSAAEECLTKNDIIFANRPRLLAGKYLGYNHTSLAWAPYSDHWRNLRRIASLEILSSHRLQMLSGIRSDEV
RSVVRRLSRASADDRVDMKKVFFELMLNVMMRMIAGKRYYGENVAEVEQGTRFREIVVETFLLSGATNMGDELPI
LNWVGVTGSEKRLMALQKKRDAFMQELIEEHRRGMGIDNGDSDEQGEKKKTMIAVLLSLQETEPDYYKDEIIRGI
TLVLLAAGTDTSAGTMEWALSLLLNNPEVLKKAQIEIDNKVGQNRLVNESDIADLPYLRCILNETERMFPVGPLL
LPHESSEDCTVGGFHIPRGTMLMINLWAIQNDPKIWEDPRKFKPERFEGLEGVRDGFKLMPFGSGRRGCPGEGLA
MRMLGFTLGSLIQCFDWERVGKDLVDLTEGPGLTMPKAQPLVAKCRPRATMLNLLSQI
Coding nucleotide sequence of putative camptothecin hydroxylase Ca32245
from Camptotheca acuminata
SEQ ID NO: 29
ATGGAGAAGTTGTACTACTGCCTTGCTCTTCTACTATCAGTTCTTCTCATATTCAAACATTTCTTCCATCATAGA
ACAAAGTTACCACCAAGTCCATTTGCTCTTCCTATCATCGGCCATCTCCATCTCATCAGGAATTCTTTCCATCAA
ATACTAGAGTGCTTGGCATCACAATATGGTCCAATTTTATTCCTCAAAGTTGGAATCCGCTCTATTCTTGTTGTG
TCGTCTCCATCCGTTGTTGAGGAATGTTTTACTAAGAATGATATTATATTTGCAAACCGTCCTCGGAATATGCTT
TCAGATATCTCTAGTTATAATTATAGTACGATCGCATGGGCTCCATATGGTCATTACTGGCGGAGCCTCCGCCGC
CTTACTGTTGTTGAATTCTTCTCATTGAATAGCCTCCAGAAGTCTTCTAACATCCGTGAAGAGGAAATTCATAAC
CTTCTCTCTCACCTCTTCAAATTCTCAAAAAGTGGAACTCAAAAAGTCCAGTTGAAATATTGGTTCTCTCTATTG
ACTTTCAATATAATAACGAGGCTGGTAGCTGGGAAGCGGTGTGTTAGAGATGCGGTTGCAGGCAAGGATTTGGGG
AAACAAATTCTTGAAGAGCTCAAGGGGAAGTTCGTTTCGAACATGCCATTGAATATGTGTGATTTCTTTCCAATT
TTGAGGTGGTTTGGTTACAAAGGGCTGGAGAAAAGTCTGATTATGTTGCTGCAGAAGGAGAGAGATGAATTCTTG
CAGGGTTTGATAGATGAGGTTAGACGAAAGAGAACCTGTTCTGCCAATATCAATATTGTAACAAACAGAGCAAAG
ACAACATTGATTGAAACTCTCTTGTCCCTCCAAGAATCAGAACCTGACTTCTTTTCTGATACTGTCATCAAAAGT
ATCATTTCAGTCATGTTTTTTGCAGGGCCGGAAACATCAGCAATTACTCTGGAATGGGCAATATCGCTTCTTCTA
AATAATCCAGAGGTACTGGGGAAGTTAAGAGCAGAGATTGATGATCATGTTGGACATGGACGCCTTCTAGATGAC
TCGGATCTTGTGAAGCTTCCCTATCTCCGTTGCATCATCAATGAGACCCTCAGATTATATCCTCCGGCACCACTT
CTATTACCACGTTGTTCATCAGAAGATTGCACTGTTGGGGGATATGAAATACCACAAGGTACAATTCTGTTGGTG
AATGCTTGGGCCATGCATAGAGATCCCAAGTTGTGGGAGGAGCCAACCAAGTTCAAGCCTGAGAGATTTGAAGGC
ATGGAAGGGAGAGAAGGGTACAAATTTATTCCATTTGGAGTTGGGAGAAGAGCTTGTCCAGGTGCTAGAATGGGC
ATCTGGACAGTTTCACTGGCTTTGGGTGCTCTTGCTCAGTGTTTTGAATGGGAAAAGGTTGTGGAGGATAAAATG
GAGATGAGCCAGGGTCGACTAACTATGTCCAAGGCCGAGTCTTTGGAGGCTCTGTGTATTCCACGCCACAGTGCA
ATGACACTCCTCTCCCAGCTTGAAGACACTTCCTTTATTTAG
Amino acid sequence of putative camptothecin hydroxylase Ca32245 from
Camptotheca acuminata
SEQ ID NO: 30
MEKLYYCLALLLSVLLIFKHFFHHRTKLPPSPFALPIIGHLHLIRNSFHQILECLASQYGPILFLKVGIRSILVV
SSPSVVEECFTKNDIIFANRPRNMLSDISSYNYSTIAWAPYGHYWRSLRRLTVVEFFSLNSLQKSSNIREEEIHN
LLSHLFKFSKSGTQKVQLKYWFSLLTFNIITRLVAGKRCVRDAVAGKDLGKQILEELKGKFVSNMPLNMCDFFPI
LRWFGYKGLEKSLIMLLQKERDEFLQGLIDEVRRKRTCSANINIVTNRAKTTLIETLLSLQESEPDFFSDTVIKS
IISVMFFAGPETSAITLEWAISLLLNNPEVLGKLRAEIDDHVGHGRLLDDSDLVKLPYLRCIINETLRLYPPAPL
LLPRCSSEDCTVGGYEIPQGTILLVNAWAMHRDPKLWEEPTKFKPERFEGMEGREGYKFIPFGVGRRACPGARMG
IWTVSLALGALAQCFEWEKVVEDKMEMSQGRLTMSKAESLEALCIPRHSAMTLLSQLEDTSFI

### Example 2: Methods

Identification and cloning of Candidates.

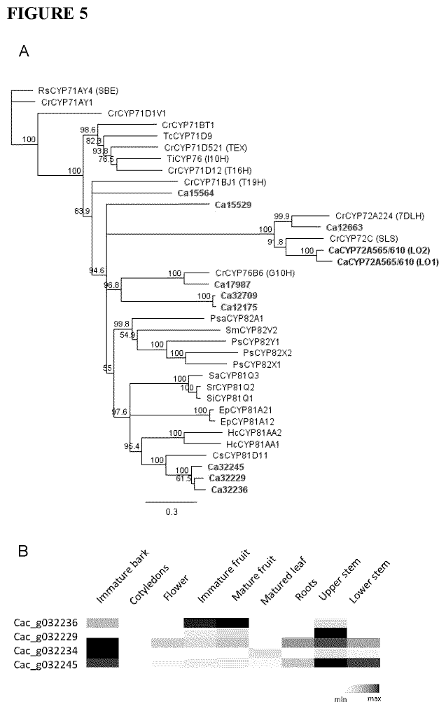

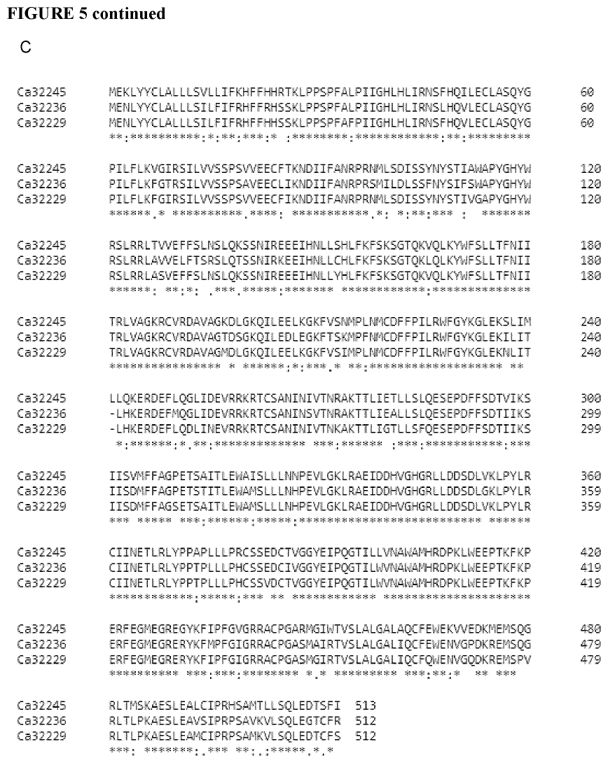

Publicly available transcriptomic and metabolomic data of seven different organs of Camptotheca acuminata (http ://medicinalplantgenomics.msu. edu/contacts.shtml) were filtered for contigs with FPKM (fragments per kilobase of exon per million fragments mapped) expression values higher than zero for more than half of the organs (FPKM expression values of zero for more than half of the treatments or with zero expression variance across the samples were removed). Self-organizing maps were applied and visualized in R (RStudio 1.0.136, RStudio, Inc) with the Kohonen package as reported before (Dang et al 2018, Nature Chemical Biology 14, 760-763). The map was assigned to give about 50 contigs per node. Cytochrome P450 (CYP450) candidates in the same nodes or neighbouring nodes with similar expression patterns with previously reported genes were selected for cloning and testing for activity. Nine CYP450 candidates belonging to different CYP450 families, including CYP71, CYP72, CYP76, CYP81 and CYP82, were identified. The full-length coding regions of CYP450s candidates were amplified using cDNA derived from total RNA of C. accuminata stems and leaves using Platinium™ SuperFi™ PCR Mastermix (Thermofisher) with appropriate primers (Table 2). Since Ca32229, Ca32236 and Ca32245 share very high sequence identity (FIG. 5), especially at the N-terminus, it is difficult to amplify individual sequences specifically. The genes were thus synthesized by Twist BioSciences (CA, USA) based on the available transcriptome (Zhao et al 2017, GigaScience 6, 1-7).

### Protein Expression

For heterologous expression of Flag-tagged CYP450s in yeast (Saccharomyces cerevisiae), the full-length coding region of each CYP450 candidate was cloned between SpeI and NcoI restriction sites of MCS1 of the dual plasmid pESC-Leu2d with a cytochrome P450 reductase (CPR) in MCS2 (Dang et al 2018, Nature Chemical Biology 14, 760-763; Rot al 2008, BMC biotechnology 8, 83) yielding pESC-Leu2d::CYP/CPR using In-Fusion cloning system (Takara Clontech). The resulted pESC-Leu2d::CYP/CPR was transformed to the protease-deficient yeast strain YPL 154C:Pep4KO, and yeast harbouring pESC-Leu2d::CPR was used as the negative control. To optimize HCPT production, Δerg6 Δtop1 yeast double mutant strain SMY75-1.4A43 was used, which was previously generated to allow better penetration of, and improved resistance to, topoisomerase I inhibitors such as CPT. The conditions for yeast culture, microsome preparation, and immunoblot analysis are further described below.

### Enzyme Assays

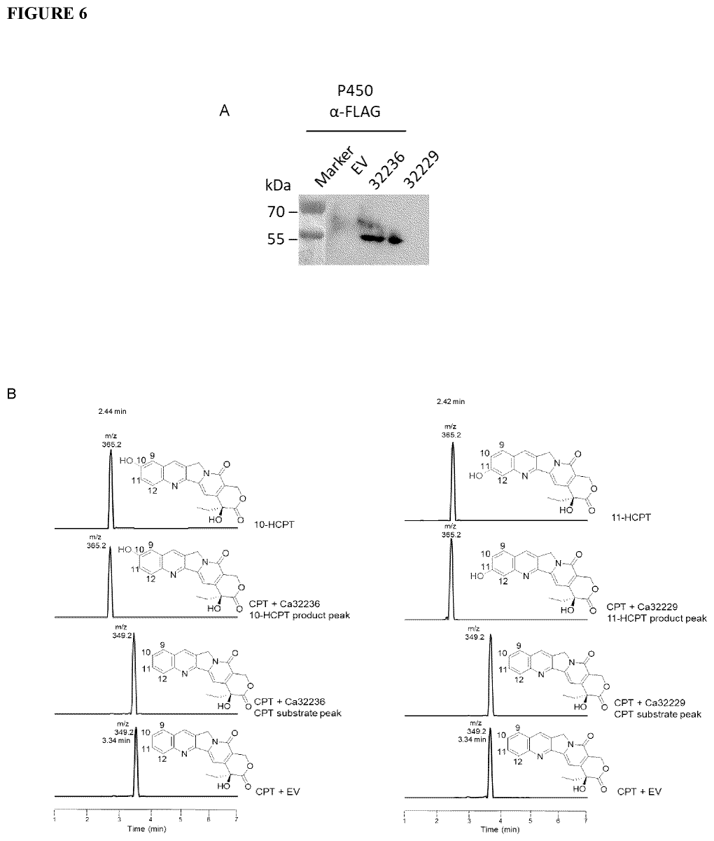

For screening in vivo CPT oxidation activities, 10 μM CPT was fed to 100-μL, cultures of YPLC 154C:Pep4KO yeast transformed with the vector for 48 h. The culture volume can be scaled up to 2 L with the camptothecin concentration up to 50 μM to produce sufficient products for structural characterization and/or semi-synthesis of camptothecin derivatives. Standard in vitro assays were performed at 30 C for 1 hour in 100 μL of 100 mM HEPES-NaOH (pH 7.5) containing 10 mg of total microsomal proteins, 50 μM substrate (FIGS. 6) and 250 μM NADPH on a gyratory shaker with agitation (750 rpm). Reactions were stopped by adding 800 μL methanol. The reaction mixture was extracted twice with methanol to precipitate and remove proteins. The supernatant was subjected to LC-MS/MS analysis

### Plants and Chemicals

Camptotheca acuminata cuttings were obtained from Quarryhill Botanical Garden (California, USA) and the Huntington Library, Art Collections, and Botanical Gardens (California, USA). The cuttings were snap-frozen upon receipt for RNA isolation. Secologanin, ajmaline, tetrahydroalstonine, serpentine, and yohimbine were purchased from Northernchem Inc. (Ontario, Canada). All other chemicals were of analytical grade from Sigma-Aldrich.

### Phylogenetic Analysis

Unrooted neighbour-joining phylogenetic tree for CYP450 candidates from this study and other reported CYP450s from other organisms were performed using the Geneious Tree Builder program in the Geneious software package (Biomatters). The names, abbreviations and GenBank accession numbers of the included sequences are: C. acuminata CPT 10-hydroxylase, CaCPT10H, 0K631678; C. acuminata CPT 11-hydroxylase, CaCPT11H, OK631675; C. acuminata Ca32245, MN631049; Arabidopsis thaliana CYP81D1, AtCTP81D1, NP_568533.2; A. thaliana CYP81F1, AtCTP81F1, 065790.2; A. thaliana CYP81H1, AtCTP81H1, NC_003075.7; A. thaliana CYP81K1, AtCTP81K1, NC_003076.8; Catharanthus roseus alstonine synthase, CrCYP71AY1, KF309243.1; C. roseus tabersonine 16-hydroxylase, CrCYP71D12, FJ647194.1; C. roseus geraniol 10-hydroxylase, CrG10H, Q8VWZ7.1; C. roseus 7-deoxyloganic acid 7-hydroxylase, Cr7DLH (CYP72A224), AGX93062.1; C. roseus CYP71BT1, AHK60840.1; C. roseus secologanin synthase, CrSLS, Q05047; C. roseus tabersonine 19-hydroxylasem, CrCYP71BJ1 (T19H), ADZ48681; C. roseus geissoschizine oxidase, CrCYP71D1V1, JN613015.1; C. roseus tabersonine 16-hydroxylase, ACM92061; C. roseus tabersonine 6,7-epoxidase, CrCYP71D521, AVH80640; Camellia sinensis CYP81D11, XP_028101205.1; Echinochloa phyllopogon CYP81A12, BA073908.1; Hypericum calycinum CYP81AA1, ANC33509.1; Rauwolfia serpentine sarpagan bridge enzyme, RsSBE, P0D013.1; Sesamum alatum CYP81Q3, BAE48236.1; Papaver somniferum CYP82X1, AFB74614.1; P. somniferum CYP82Y1 AFB74617.1; P. somniferum CYP82X2, AFB74617.1; Sesamum indicum CYP81E8, NP_001306620.1; Salvia miltiorrhiza CYP82V2, KP337709.1L; Sesamum radiatum CYP81Q2, AB194715.1; Theobroma cacao CYP71D9, XM_018120397.1; and Tabernanthe iboga ibogamine 10-hydroxylase (I10H), TiCYP76, MH454074.1.

### Yeast Culture, Microsome Preparation and Immunoblot Analysis

For routine yeast culture, the transgenic yeast strain was inoculated in 2 mL of synthetic complete (SC) medium lacking leucine (SC-Leu) containing 2% (w/v) glucose and cultured overnight at 30 oC and 250 rpm. The culture was subsequently diluted 100-fold to an OD600 of 0.05 in SC-Leu supplemented with 2% (w/v) glucose and cultured for 16 hr. Yeast was then harvested and sub-cultured for 24 hr in YPA medium containing 2% (w/v) galactose to induce the production of recombinant CYP450s. Yeast cells were harvested by centrifugation and lysed for 2 min using a micro-bead beater (VWR) and 500-μm diameter glass beads in TES (0.6 M sorbitol in TE) buffer. The resulting lysate was subsequently centrifuged at 10,000 g for 15 min at 4 C. The supernatant was then transferred to a new tube and centrifuged at 40,000 g for 60 min at 4 C. Finally, the pellet containing microsomes was resuspended with TEG buffer (20% (v/v) glycerol in TE). Expression of Ca32229 and Ca32236 was confirmed by immunoblot analysis of microsomal fractions prepared from S. cerevisiae cultures harbouring the pESC-Leu2d::CPR/Ca32229 and pESC-Leu2d::CPR/Ca32236 vectors using α-FLAG M2 antibodies (ThermoFisher Scientific) detectable with SuperSignal West Pico Chemiluminescent Substrate (ThermoFisher Scientific) to probe epitope-tagged recombinant proteins (FIG. 6).

### LC-MS/MS Analysis

Enzyme assays were analyzed by ultra-performance liquid chromatography (UPLC) on a Xevo TQ-S Cronos Triple Quadrupole Mass Spectrometry (Waters). For all studies, chromatography was performed on an XBridge BEH XP (10×2.1 mm, 1.7 μm) column at a flow rate of 0.6 mL.min−1. The column was equilibrated in solvent A (0.1% formic acid) and the following elution conditions were used: 0 min, 5% B (100% acetonitrile); from 0 to 3.5 min, 35% B; from 3.5 min to 3.75 min, 100% B; 3.75 min to 4.75, 100% B; 4.75 to 6 min, 5% B to re-equilibrate the column. Data were analyzed with MassLynx and TargetLynx (Waters).

For high-resolution MS (HRMS) analysis, new compounds were subjected to the Agilent 1290 Infinity system connected to the Agilent 6530 Quadrupole Time-of-Flight (QTOF). Chromatography was performed on an XBridge BEH XP (10×2.1 mm, 1.7 μm) column at a flow rate of 0.6 mL.min−1. The column was equilibrated in solvent A (0.1% formic acid) and the following elution conditions were used: 0 min, 5% B (100% acetonitrile); from 0 to 3.5 min, 35% B; from 3.5 min to 3.75 min, 100% B; 3.75 min to 4.75, 100% B; 4.75 to 6 min, 5% B to re-equilibrate the column. Data were analyzed with Mass Hunter (Agilent Technologies).

### Conversion Rate and Yield Calculation

A calibration curve using camptothecin from 0-50 nM was made for quantification. Peaks areas of LC-MS chromatograms were calculated using MassLynx and TargetLynx from Waters and normalized. The amount of substrate consumption, product formation, conversion, and total product yield was quantified using corresponding calibration curves.

### Semi Preparative HPLC and NMR Analyses for Structure Elucidation

A scaled-up yeast in vivo assay with CPT and 7-ethyl-CPT substrates were performed to produce sufficient product quantities of HCPTs and 7-ethyl-HCPT for NMR analysis. The supernatant of the assays was obtained by centrifugation. The crude containing HCPT and 7-ethyl-HCPT in the supernatant were collected by liquid-liquid extraction with ethyl acetate and chloroform, respectively. Product purification from the concentrated sample was performed by a semi-preparative HPLC system with Kinetex® 5 μm EVO C18 100 Å, 10×250 mm column at a flow rate of 1.5 mL.min−1. The column was equilibrated in solvent A (water, 0.1% formic acid) and solvent B (0.1% formic acid in acetonitrile). Then, the following elution conditions were used: 0 min, 10% B; from 0 to 5 min, 20% B; from 5 to 25 min, 70% B; from 25 to 27 min, 90% B; from 27 to 30 min, 90% B; from 30 to 31 min; 10% B; from 31 to 34 min, 10% B to re-equilibrate the column. Approximately 1 mg of each product was independently dissolved in 600 μL DMSO-d6 and subjected to 1H NMR analysis on Bruker Avance 600 NMR spectrometer. 1D-TOCSY NMR technique (50 ms spin-lock time) were used afterwards to analyze the overlapped aromatic protons signals with irradiation frequency set at 8.02 ppm. The ¹H NMR spectra were analyzed and compared with those of standards and literature for known compounds.

### Scale-Up and Purification of New Compounds for Chemoenzymatic Synthesis of Hydroxycamptothecin Derivatives

To generate sufficient amounts of HCPTs (10 and 11HCPT) and 7-ethyl-HCPT (7-ethyl-10 and 11HCPT) for the synthesis of topotecan, irinotecan and other compounds, enzymatic reactions was scaled up. The transgenic yeast strain was inoculated in 2 mL of synthetic complete medium lacking leucine (SC-Leu) containing 2% (w/v) glucose and cultured overnight at 30° C. and 275 rpm. The culture was subsequently diluted to an OD600 of 0.05 in SC-Leu supplemented with 2% (w/v) glucose and cultured for 16 hr. The yeast was then harvested and sub-cultured for 48 hr in YPA medium containing 2% (w/v) galactose, and 10% glycerol to induce the production of recombinant CYP450s. CPT or 7-ethyl-CPT substrate was fed directly into the culture to reach a final concentration of 50 μM as soon as the yeast was switched from SC-Leu to YPA medium. After 48-hr inoculation, a conversion rate of approximately 70% from CPT or 7-ethyl-CPT to its hydroxylated product was obtained and confirmed by LCMS analysis. The supernatant was collected by centrifugation at 4000 rpm, for 5 minutes. HCPT and 7-ethyl-HCPT were extracted out of reaction matrix by liquid-liquid extraction with ethyl acetate and chloroform, respectively. The solvent was removed by using a rotary evaporator to obtain crude HCPT and 7-ethyl-HCPT substrates for chemical synthesis to topotecan and irinotecan. HCPT and 7-ethyl-HCPT were purified by semi-preparative HPLC prior to the synthesis of derivatives.

Semi-Synthesis of Topotecan and Topotecan-11 (12-[(dimethylamino)methyl]-11HCPT)

Fifteen mg of solid N,N-dimethylmethyleneiminium chloride was added into an empty 4 mL reaction flask. Six mg of HCPT substrates from the enzymatic reaction was dissolved by 1 mL isopropanol:chloroform (1:1) and transferred into the reaction flask. Two μL triethylamine was added into the mixture then the reaction mixture was magnetically stirred at room temperature for 24 hr. Then, the mixture was acidified to pH 3-4 with 1 N HCl1. The reaction mixture was analyzed by LC-MS/MS method to identify the topotecan product. The solvent in the reaction mixture was removed to dryness in vacuo. The dried reaction mixture was dissolved in methanol and the final product was purified by semi-prep HPLC to yield approximately 4 mg dried product. The dried product was dissolved in DMSO-d6 and subjected to 1H NMR analysis on Bruker Avance 600 NMR spectrometer in order to elucidate the structure of the final product.

Semi-Synthesis of Irinotecan and Irinotecan-11 (7-ethyl-11-[4-(1-piperidino)-1-piperidino]carbonyloxyCPT)

Six mg of solid 4-piperidinopiperidine-1-carbonyl chloride was added into an empty 4 mL reaction flask. One mg of 7-ethyl-HCPT substrates from the enzymatic reaction was dissolved by 200 μL pyridine and transferred into the reaction flask. The reaction mixture was magnetically stirred at room temperature for 2 hr. The reaction mixture was analyzed by LC-MS/MS method to detect the irinotecan product. Pyridine was removed by rotatory evaporator after 2 hr. The dried crude mixture was dissolved in 300 μL water. Then 1.5 mL dichloromethane was used to extract the irinotecan product out of the mixture. Dichloromethane layer was dried in vacuo to obtain 1.5 mg dried product. The dried product was dissolved in DMSO-d6 and subjected to ¹H NMR analysis on a Bruker Avance 600 NMR spectrometer in order to elucidate the structure of the final product.

### Semi-Synthesis of Brominated HCPTs

An amount of 15 mg of solid N-bromosuccinimide (NBS) was added into an empty 4 mL reaction flask. Three mgs of dried HCPT substrates from the enzymatic reaction were dissolved by 200 μL DMSO (pre-cooled at 4° C.). After that, the substrate was transferred into the flask containing N-bromosuccinimide on ice. The mixture was magnetically stirred at room temperature in the dark for 2 hr. The reaction progress was analyzed by LC-MS/MS method to detect the brominated HCPT product. Then, the reaction mixture was transferred into 5 mL cold water, the pH of the mixture was adjusted to 3-4 with 1 N HCl₂. Water and organic solvent were removed by GeneVac evaporator with a temperature below 40° C. The dried reaction mixture was then dissolved in methanol, and the pure brominated product was purified by semi-prep HPLC to obtain 1.1 mg dried product. The dried product was dissolved in DMSO-d6 and subjected to ¹H NMR analysis on a Bruker Avance 600 NMR spectrometer to determine the position of the bromine substituent position.

### Example 3: Identification of Cytochrome P450 Monooxygenase Enzymes

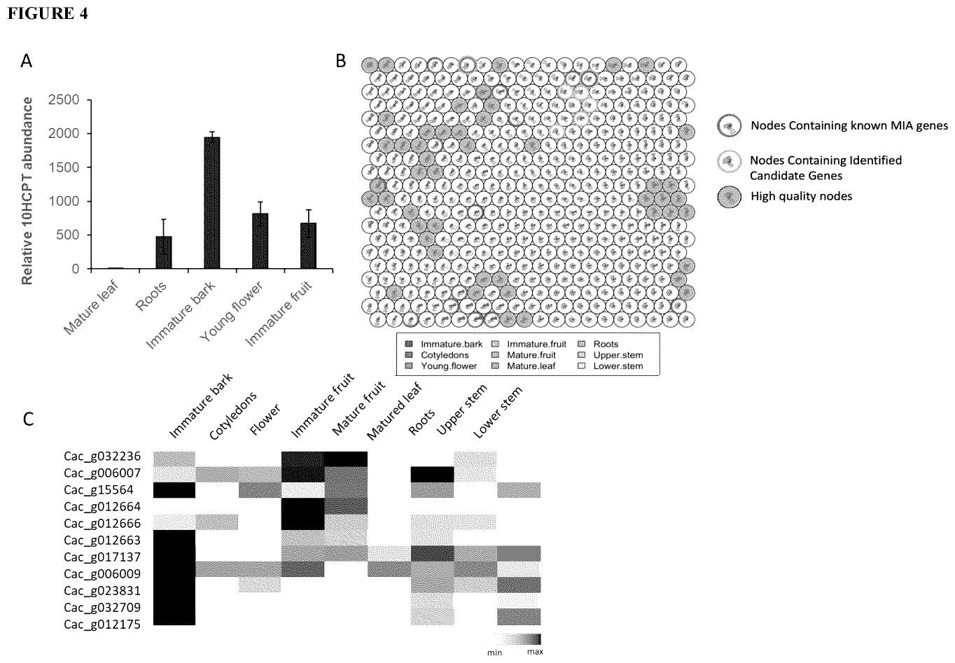

Targeted metabolomics studies of C. acuminata showed that while CPT accumulates in young leaves, its oxidized derivatives (HCPTs) are primarily found in stems, fruits and bark (FIG. 4A). Therefore, it was speculated that C. acuminata's genes encoding for enzymes involved in converting CPT to HCPTs would be highly expressed in stems, fruits and bark. The search was focused on CPT oxidative enzymes within the cytochrome P450 monooxygenases (CYP450s) as they are the main players in the oxygenation of plant specialized metabolites (Nguyen and Dang 2021, Frontiers in Plant Science 12).

Using the available C. acuminata transcriptome and genome data (Zhao et al. 2017; Gongora-Castillo et al 2012, PLoS ONE 7) for a self-organizing map analysis (Hur et al. 2013 Natural Product Reports 30, 565) (FIG. 4B), nine candidates were identified that show similar expression patterns with those of other MIA biosynthetic genes and 10HCPT accumulation (FIG. 4C). These candidates belong to different CYP450 clades (FIG. 5A).

To test for enzymatic activities, these CYP450 candidates-coding sequences were cloned into the galactose-inducible dual expression vector pESC-Leu2d with a redox partner cytochrome P450 reductase (CPR) (Ro et al 2008, BMC biotechnology 8, 83) using primers as shown in Table 3.

TABLE 3
Primers used to assemble CYP450 candidates in pESC-leu2d expression vector

| | | | Insert size |
|---|---|---|---|
| Vector name | Forward primer (5′ to 3′) | Reverse primer (5′ to 3′) | (bp) |
| pESC-Leu2d-32245 | CAC TAA AGG GCG GCC AAC AAA ATG | CAC TAA AGG GCG GCC AAC AAA ATG | 1542 |
| | GAG AAG TTG TAC TAC TGC | GAG AAG TTG TAC TAC TGC CT | |
| | (SEQ ID NO: 31) | (SEQ ID NO: 32) | |
| | CAC TAA AGG GCG GCC AAC AAA | ATC CAT CGA TAC TAG | |
| pESC-Leu2d-32236 | ATGGAGAACTTGTACTACTGCCT | ACGGAAACAAGTGCCTTCA | 1533 |
| | (SEQ ID NO: 33) | (SEQ ID NO: 34) | |
| pESC-Leu2d-32245 | CAC TAA AGG GCG GCC AAC AAA ATG | ATC CAT CGA TAC TAG | 1542 |
| | GAG AAG TTG TAC TAC TGC | AATAAAGGAAGTGTCTTCAAGCTGG | |
| | (SEQ ID NO: 35) | (SEQ ID NO: 36) | |
| | CAC TAA AGG GCG GCC AAC AAA | ATC CAT CGA TAC TAG | |
| pESC-Leu2d-32229 | ATGGAGAACTTGTACTACTGCCT | ACTGAAACAAGTGTCTTCAAGCTG | 1536 |
| | (SEQ ID NO: 37) | (SEQ ID NO: 38) | |

Ten μM CPT was fed to 100-4, cultures of the Saccharomyces cerevisiae yeast transformed with the vector for 48 h. Only yeast harbouring pESC-Leu2d::CPR/Ca32236 showed the consumption of CPT and the formation of a new product with a mass (m/z 365.2), an increase in 16 amu as compared to that of the substrate (m/z 349.2) and retention time corresponding to 10HCPT (FIG. 2A). No enzymatic product was observed when CPT was incubated with yeast expressing empty vector or any of the other candidates. Similarly, in vitro assays with microsomal fractions of yeast transformed with pESC-Leu2d::CPR/Ca32236 showed that in the presence of NADPH, CPT was consumed, and a new product with m/z 365.2 was formed as evidenced by LC-MS analysis (FIG. 6), signifying an oxidation event.

In addition to 10HCPT, C. accuminata also produces a limited amount of 11HCPT. Using Ca32236 as a query, other putative CPT oxidative enzymes in C. acuminata transcriptomes were identified namely Ca32234, Ca32229, and Ca32245, sharing 80-93% amino acid identity (FIG. 5B). Using the same in vivo assay system (Wall et al 1986, Journal of Medicinal Chemistry 29, 1553-1555) (FIG. 1), it was found that cultures of yeast harbouring a plasmid with one of these candidates, pESC-Leu2d::CPR/Ca32229, produced a compound with the same m/z value (365.2) of the 10HCPT derivative but a different retention time in LC-MS analysis (FIG. 2B).

### Example 4: Activity of Cytochrome P450 Monooxygenase Enzymes

To rigorously confirm the structure of the compounds produced by Ca32229 and Ca32236, the transgenic yeast cultures were upscaled to 1 L. Approximately 5-8 mg of the two products were purified and subjected to ¹H, ³C and 1D-TOCSY NMR analyses. The NMR data confirmed that both Ca32236 and Ca32229 catalyzed hydroxylations of CPT (FIGS. 7 and 8). Ca32236 hydroxylated CPT at C-10 to produce 10HCPT (FIGS. 2 and 7, Example 9) while Ca32229 catalyzed the hydroxylation at C-11 to yield 11HCPT (FIGS. 2 and 8, Example 9). Ca32236 and Ca32229 were thus named CPT 10-hydroxylase (CPT10H) and CPT 11-hydroxylase (CPT11H), respectively. NMR data of the substrate camptothecin was also included for comparison (FIG. 8). No other products were detected.

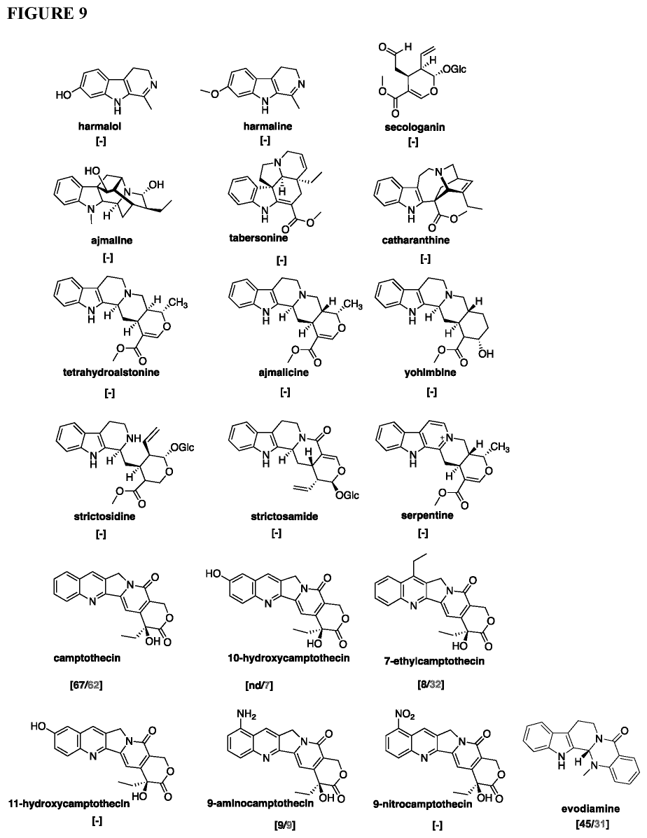

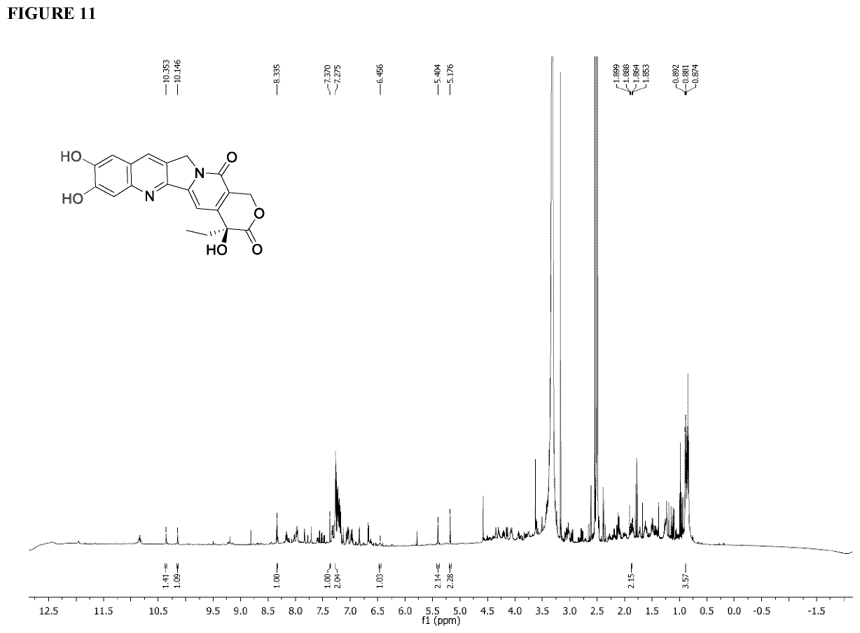

Next, to investigate the substrate scopes of the newly found enzymes, the two enzymes with 18 alkaloids were assayed representing different MIA structural subgroups including β-carbolines, ajmaline, heteroyohimbines, and quinolines (FIG. 9). Results showed that the substrate range of CPT10H and CPT11H is restricted to the CPT scaffold. Intriguingly, both CPT10H and CPT11H accepted the commercially available 7-ethylcamptothecin (7-ethyl-CPT) to produce the antineoplastic drug SN-38 (7-ethyl-10HCPT) (FIG. 10A) and its isomer 7-ethyl-11HCPT (FIGS. 8, 9, and 10B, Example 9), respectively. CPT11H also accepted 10HCPT to produce low amounts (7% conversion) of 10,11-dihydroxyCPT (FIGS. 9, 10C, and 11, Example 9). However, 11HCPT was not accepted by CPT10H (FIG. 10D). Of note, CPT10H and CPT11H also converted 9-amino-CPT to two new products (FIGS. 9 and 12). The limited availability of 9-amino-CPT and low conversion rate (9%) precluded the product structure elucidation by NMR spectroscopy. It is speculated that the products are 9-amino-10HCPT and 9-amino-11HCPT (9A10HCPT and 9A11HCPT; FIG. 12) based on the observed m/z (380.1, an increase in 16 amu as compared to that of the substrate (m/z 364.1)) and the regio-specificity of CPT10H and CPT11H toward C-10 and C-11, respectively, on the CPT scaffold. Altogether, these enzymes could produce seven products from the CPT scaffold (Table 4A), of which 11HCPT, 10,11-dihydroxyCPT, putative 9-aminohydroxyCPTs have not been reported in any biosynthetic or synthesis studies while 7-ethyl-11 HCPT has been described elsewhere (Yoshikawa et al 2004, International Journal of Cancer 110, 921-927; Luo et al 2014 Journal of Heterocyclic Chemistry 51, 1133-113).

TABLE 4A
Hydroxylated camptothecinoid product yield by enzymatic contacting of camptothecin by
cytochrome P450 monooxygenase

| | | | | | | | Pure |
|---|---|---|---|---|---|---|---|
| | | | | | | | products |
| | | | | | | Yield of | after |
| | | Hydroxylated | | Conversion | | biotransformation | semiprep |
| | Camptothecinoid | camptothecinoid | Starting | rate | Starting | in crude extract | HPLC |
| Enzyme | substrate | product | material | (%)ª | material | (mg)^{b} | (mg) |
| Ca32236/ | CPT | 10- | 18.0 | 67 | 18.0 | 12.0 | 9.4 |
| CPT10H | | HydroxyCPT | | | | | |
| | 7-EthylCPT | 7-Ethyl-10CPT | 18 | 8 | 18 | 1.5 | 0.6 |
| | 9-AminoCPT | 9-Amino- | 18 | 9 | 18 | 1.7 | n/a^{c} |
| | | 10CPT | | | | | |
| Ca32229/ | CPT | 11- | 17 | 62 | 17 | 11.0 | 8.1 |
| CPT11H | | HydroxyCPT | | | | | |
| | 7-EthylCPT | 7-Ethyl-11- | 19 | 32 | 19 | 6.1 | 3.5 |
| | | hydroxyCPT | | | | | |
| | 9-AminoCPT | 9-amino-10- | 18 | 9 | 18 | 1.7 | n/a^{c} |
| | | hydroxyCPT | | | | | |
| | 10-HydroxyCPT | 10,11- | 18 | 11 | 18 | 2.0 | 0.6 |
| | | DihydroxyCPT | | | | | |

^{a}conversion rate calculated based on LCMS analysis
^{b}yield of biotransformation from the yeast in vivo assay was obtained from 1 L yeast culture incubated with 17 mg camptothecin starting material.
^{c}due to the low yield and low recovery rate of our semi prep system, these products couldn't be recovered for further structural elucidation

TABLE 4B
Product yield in semisynthesis of new compounds from enzymatic products

| | | | | Yield of | |
|---|---|---|---|---|---|
| | | Starting | Conversion | camptothecin | Product recovery |
| Hydroxylated | Camptothecin | material | rate | derivative | (mg) after |
| camptothecinoid | derivative | (mg) | (%)^{a} | (mg) | semiprep HPLC |
| 10-Hydroxycamptothecin | Topotecan | 6.9 | 100 | 8 | 4.0 |
| | 9-bromo-10HCPT | 10.5 | 100 | 12.75 | 4.0 |
| 7-Ethyl-10- | Irinotecan | 1.1 | 100 | 1.7 | 1.5 |
| hydroxycamptothecin | | | | | |
| 11-Hydroxycamptothecin | Topotecan-11 | 5.9 | 100 | 6.8 | 6.0 |
| | 12-bromo-11HCPT | 3.1 | 100 | 3.8 | 1.1 |
| 7-Ethyl-11- | Irinotecan-11 | 7.4 | 100 | 11.5 | 8.0 |
| hydroxycamptothecin | | | | | |

### Example 5: Optimization of Hydroxylated Camptothecin Yield

A key advantage of the cytochrome P450 monooxygenase enzymes lies in the opportunity to functionalize the inert C—H bond and to further diversify the products to obtain valuable CPT-based scaffolds. With the newly-discovered regio-selective CPT hydroxylases, it next demonstrated combinatorial enzymatic and chemical syntheses of CPT analogues topotecan and irinotecan and their 11HCPT-derived isomers from CPT (FIG. 3). First, the enzymatic conversion of CPT to HCPTs in yeast expressing CPT hydroxylases was optimized. The initial in vivo conversion rate maximized at 10% (FIG. 2), possibly because CPT is insoluble and the native yeast topoisomerase I is sensitive to CPT. Different growth conditions were investigated and optimized to achieved a yield up to 40% from transgenic yeast grown in YPA medium with 2% galactose and 10% glycerol for 48 hrs. To further increase the yield, the CPT hydroxylases was expressed in SMY75-1.4A yeast strain (Δerg6 Δtop1), which was previously engineered to allow better penetration of, and improved resistance to, topoisomerase I inhibitors such as CPT (Del Poeta et al 1999, Antimicrobial Agents and Chemotherapy 43, 2862-2868). As a result, a markedly improved conversion of CPT, up to 67% (12 mg/L of 10HCPT and 11 mg/L of 11 HCPT from 18 mg/L starting CPT in the crude extract, which yields 9.4 mg/L of pure 10HCPT and 8.1 mg/L of pure 11HCPT after further purification by semiprep HPLC) was obtained (Table 4A). This incredible in vivo enzymatic conversion rate and high regio-selectivity in mild conditions surpassed a typical chemical synthesis reaction (˜50-60%) (Kingsbury et al 1991), affording 10HCPT and 11HCPT for the following chemoenzymatic process (FIG. 3) to produce clinically essential compounds topotecan and irinotecan as well as other derivatives.

### Example 6: Chemoenzymatic Synthesis of Camptothecin Derivatives with Cytochrome P450 Monooxygenase Enzymes

Treatment of enzymatically produced 10HCPT with an appropriate iminium reagent, N,N-dimethylmethyleneiminium chloride, yielded 9-[(dialkylamino)methyl]-10HCPT, commonly known as topotecan (FIGS. 3A and 13A). When the enzymatic product 11HCPT was allowed to react with the same iminium reagent, and a total conversion to the new product 12-[(dialkylamino)methyl]-11HCPT (topotecan-11) was obtained (FIGS. 3A, 13B, and 14A, Example 9). Likewise, using the enzymatic products 7-ethyl-10HCPT and 7-ethyl-11HCPT with [1,4′]bipiperidinyl-1′-carbonyl chloride in pyridine, conversions to the clinically important drug irinotecan and its 11HCPT-derived isomer, 7-ethyl-11-[4-(1-piperidino)-1-piperidino]carbonyloxyCPT (irinotecan-11) was achieved (FIGS. 3B, 14B, and 15, Example 9). Furthermore, using a halogenated reagent such as N-bromosuccinimide on 10HCPT and 11HCPT derived from in vivo biosynthesis afforded 9-bromo-10HCPT and 12-bromo-11HCPT (FIGS. 16, 17 and 18, Example 9). All new chemoenzymatic products were confirmed by LC-MS (FIGS. 13, 15, and 16), high-resolution MS (Example 9) and NMR analyses (FIGS. 8, 11, 14, 17, and 18, Example 9). The formation of topotecan and irinotecan products was also validated on LC/MS and NMR with authentic standards (FIG. 3, 13A and 15A).

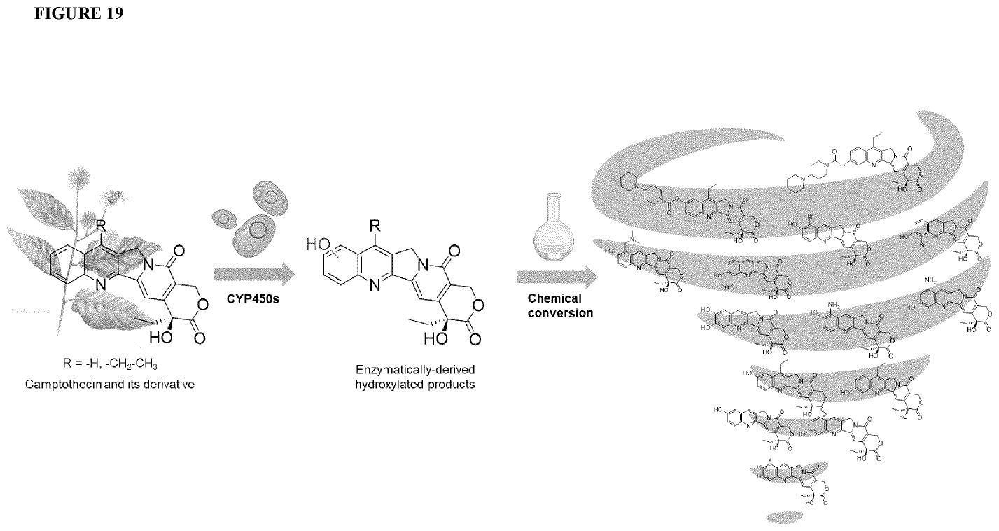

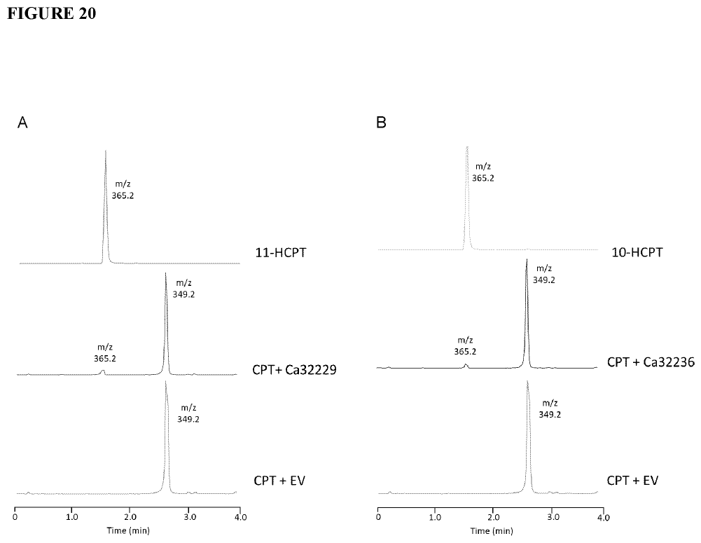

In total, biosynthesis and chemoenzymatic production of 13 CPT analogues from CPT (FIG. 19). These products encompass compounds naturally occurring in plants (10HCPT and 11HCPT) and clinically active semi-synthetic drugs (SN-38, topotecan and irinotecan). The products include four novel compounds, namely, 12-bromo-11HCPT, topotecan-11 (12-[(dimethylamino)methyl]-11HCPT), 10,11-dihydroxyCPT, and irinotecan-11 (7-ethyl-11-[4-(1-piperidino)-1-piperidino]carbonyloxyCPT), all of which are not readily accessible either from plants or via conventional chemical C—H functionalization approach. All the chemoenzymatic conversions were completed at room temperature as no substrates, or decomposition products were detected at the end (FIGS. 3, 13, 14, and 15).

### Example 7: Expression of CPTHs in Plant

For transient expression of CPTHs in N. benthamiana, the full-length coding regions were cloned into NotI restriction site of our in house pTRBO::ESC using the In-Fusion cloning system (Takara Clontech). pTRBO constructs were transformed into Agrobacterium tumefaciens GV3101 by electroporation. Transformants were selected on LB plates containing kanamycin, gentamicin and rifampicin. Cells were grown for 48 hrs at 28° C. before harvested by centrifugation. The pellet was resuspended in infiltration buffer (10 mM NaCl, 1.75 mM CaCl2, 100 μM acetosyringone) and incubated at room temperature for 2 hrs. Agrobacterium suspensions (OD600=0.1 for each strain) were infiltrated into the abaxial side of 5 week old N. benthamiana leaves with a needleless 1 mL syringe. Substrate (50 μM) and caffeine standard (100 μM) were infiltrated into the leaves 3 days post bacteria infiltration. Leaves were flash frozen in liquid N₂ and stored at −70° C. before processing. The presence of the CPTHs products, 10HCPT and 11HCPT was confirmed by LCMS analysis.

TABLE 5
Chemical compounds of the disclosure

| | Compound Cited in | | |
|---|---|---|---|
| Compound | Application | | |
| ID | IUPAC Name (if available) | Synonym or Abbreviation | Structure |
| 1 | camptothecin (19S)-19-ethyl-19- hydroxy-17-oxa-3,13- diazapentacyclo [11.8.0.0^{2,11}.0^{4,9}.0^{15,20}]henico sa-1(21),2,4,6,8,10,15(20)- heptaene-14,18-dione | CPT, camptothecin Camptothecine 7689-03-4 (S)-(+)-Camptothecin Campathecin | 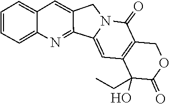 [SMILES: CCC1(O)C(=O)OCc2c1cc1n(c2=O)Cc2cc3ccccc3nc2-1] |
| 2 | 10-hydroxycamptothecin (19S)-19-ethyl-7,19- dihydroxy-17-oxa-3,13- diazapentacyclo [11.8.0.0^{2,11}.0^{4,9}.0^{15,20}]henico sa-1(21),2(11),3,5,7,9,15(20)- heptaene-14,18-dione | 10-HCPT, 19685-09-7 (S)-10- Hydroxycamptothecin Hydroxycamptothecin 10-hydroxycamptothecine | 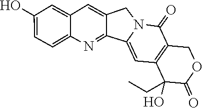 [SMILES: CCC1(O)C(=O)OCc2c1cc1n(c2=O)Cc2cc3cc(O)ccc3nc2-1] |
| 4 | topotecan (19S)-8-[(dimethylamino) methyl]-19-ethyl- 7,19-dihydroxy-17-oxa-3,13- diazapentacyclo [11.8.0.0^{2,11}.0^{4,9}.0^{15,20}Thenico sa-1(21),2,4(9),5,7,10,15(20)- heptaene- 14,18-dione | 9-[(dimethylamino) methyl]-10- hydroxycamptothecin 123948-87-8 Hycamtin Topotecan lactone Hycamptamine | 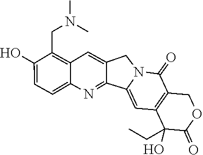 [SMILES: CCC1(O)C(=O)OCc2c1cc1n(c2=O)Cc2cc3c(CN(C)C)c(O)ccc3nc2-1] |
| 11 | 9-X-10- hydroxycamptothecin (No IUPAC designation; generic structure) | 9-X-10-HCPT | 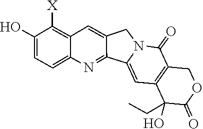 |
| 11a | 9-bromo-10- hydroxycamptothecin (No IUPAC designation) | 9-Br-10-HCPT | 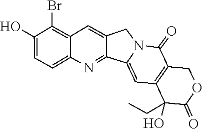 [SMILES: CCC1(O)C(=O)OCc2c1cc1n(c2=O)Cc2cc3c(Br)c(O)ccc3nc2-1] |
| 11b | 9-iodo-10- hydroxycamptothecin (No IUPAC designation) | 9-I-10-HCPT | 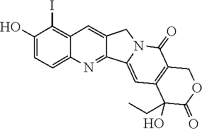 [SMILES: CCC1(O)C(=O)OCc2c1cc1n(c2=O)Cc2cc3c(I)c(O)ccc3nc2-1] |
| 7 | 7-ethyl-10- hydroxycamptothecin (19S)-10, 19-diethyl- 7,19-dihydroxy-17-oxa-3,13- diazapentacyclo [11.8.0.0^{2,11}.0^{4,9}.0^{15,20}] henicosa- 1(21),2,4(9),5,7,10,15(20)- heptaene-14,18-dione | SN-38 7-Ethyl-10-hydroxy- camptothecin 86639-52-3 SN 38 SN 38 lactone | 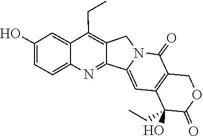 [SMILES: CCc1c2c(nc3ccc(O)cc13)-c1cc3c(c(=O)n1C2)COC(=O)[C@]3(O)CC] |
| 3 | irinotecan [(19S)-10,19-diethyl- 19-hydroxy-14,18- dioxo-17-oxa-3,13- diazapentacyclo [11.8.0.0^{2,11}.0^{4,9}.0^{15,20}] henicosa- 1(21),2,4(9),5,7,10,15(20)- heptaen-7-yl] | 97682-44-5 (+)-Irinotecan Camptosar Irinotecanum | 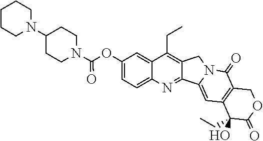 [SMILES: CCc1c2c(nc3ccc(OC(=O)N4CCC(N5CCCCC5)CC4)cc13)-c1cc3c(c(=O)n1C2)COC(=O)[C@]3(O)CC] |
| | 4-piperidin-1-ylpiperidine- | | |
| | 1-carboxylate | | |
| 12 | 10,11- dihydroxycamptothecin (No IUPAC designation) | 10,11-HCPT | 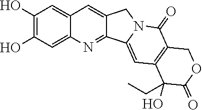 [SMILES: CCC1(O)C(=O)OCc2c1cc1n(c2=O)Cc2cc3cc(O)c(O)cc3nc2-1] |
| 9 | 12-[(dimethylamino) methyl]-11- hydroxycamptothecin (No IUPAC designation) | topotecan-11 topotecan 11- hydroxy-isomer | 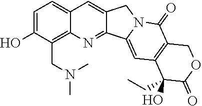 [SMILES: CC[C@@]1(O)C(=O)OCc2c1cc1n(c2=O)Cc2cc3ccc(O)c(CN(C)C)c3nc2-1] |
| 5 | 11-hydroxycamptothecin (19S)-19-ethyl-6,19- dihydroxy-17-oxa-3,13- diazapentacyclo [11.8.0.0^{2,11}.0^{4,9}.0^{15,20}]henico sa-1(21),2(11),3,5,7,9,15(20)- heptaene-14,18-dione | 11-HCPT 11-Hydroxycamptothecin 68426-53-9 (11-hydroxy camptothecin CHEMBL39011 | 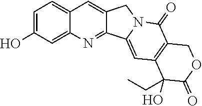 [SMILES: CCC1(O)C(=O)OCc2c1cc1n(c2=O)Cc2cc3ccc(O)cc3nc2-1] |
| 14 | X-11-hydroxycamptothecin (No IUPAC designation; generic structure) | X-11-HCPT | 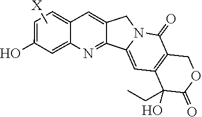 |
| 15 | 9-bromo-11- hydroxycamptothecin (No IUPAC designation) | 9-Br-11-HCPT | 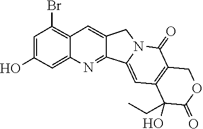 [SMILES: CCC1(O)C(=O)OCc2c1cc1n(c2=O)Cc2cc3c(Br)cc(O)cc3nc2-1] |
| 16 | 9-iodo-11- hydroxycamptothecin (No IUPAC designation) | 9-I-11-HCPT | 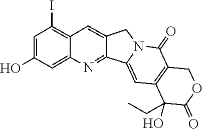 [SMILES: CCC1(O)C(=O)OCc2c1cc1n(c2=O)Cc2cc3c(I)cc(O)cc3nc2-1] |
| 8 | 7-ethyl-11- hydroxycamptothecin (No IUPAC designation) | 7-Ethyl-11-hydroxy- camptothecin 7-Ethyl-11-hydroxy-CPT | 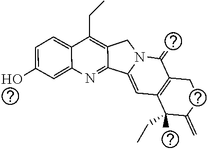 [SMILES: C=C1OCc2c(cc3n(c2=O)Cc2c-3nc3cc(O)ccc3c2CC)[C@@]1(O)CC] |
| 10 | Irinotecan-11 (No IUPAC designation) | Irinotecan ortho isomer 7-ethyl-11-[4-(1-piperidino)-1- piperidino] carbonyloxycamptothecin | 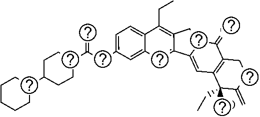 [SMILES: C=C1OCc2c(cc3n(c2=O)Cc2c-3nc3cc(OC(=O)N4CCC(N5CCCCC5)CC4)ccc3c2CC)[C@@]1(O)CC] |
| 6 | 7-ethylcamptothecin (19S)-10,19-diethyl- 19-hydroxy-17-oxa- 3,13-diazapentacyclo [11.8.0.0^{2,11}.0^{4,9}.0^{15,20}] henico sa-1(21),2,4,6,8,10,15(20)- heptaene-14,18-dione | 7-ethyl-CPT 7-Ethylcamptothecin 78287-27-1 7-Ethyl camptothecin (S)-4,11-Diethyl-4-hydroxy-1H- pyrano[3',4':6, 7]indolizino[1,2- b]quinoline-3,14(4H,12H)-dione SN-22 | 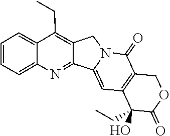 [SMILES: CCc1c2c(nc3ccccc13)-c1cc3c(c(=O)n1C2)COC(=O)[C@]3(O)CC] |
| 17 | 12-bromo-11- hydroxycamptothecin (No IUPAC designation) | | 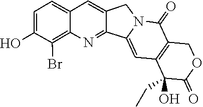 [SMILES: CC[C@@]1(O)C(=O)OCc2c1cc1n(c2=O)Cc2cc3ccc(O)c(Br)c3nc2-1] |
| 18 | 9-amino-10- hydroxycamptothecin (No IUPAC designation) | | 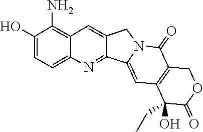 [SMILES: CC[C@@]1(O)C(=O)OCc2c1cc1n(c2=O)Cc2cc3c(N)c(O)ccc3nc2-1] |
| 19 | 9-amino-11- hydroxycamptothecin (No IUPAC designation) | | 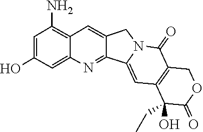 [SMILES: CC[C@@]1(O)C(=O)OCc2c1cc1n(c2=O)Cc2cc3c(N)cc(O)cc3nc2-1] |
| 20 | 9-[(dimethylamino) methyl]-11- hydroxycamptothecin | topotecan-11 isomer | 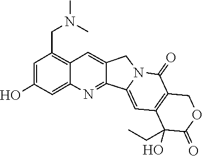 [SMILES: CCC1(O)C(=O)OCc2c1cc1n(c2=O)Cc2cc3c(CN(C)C)cc(O)cc3nc2-1] |

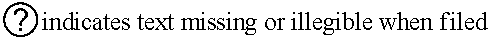

### Example 9: Spectroscopic and Spectrometric Analyses of Disclosed Compounds

10-hydroxycamptothecin: ¹H-NMR (600 MHz, DMSO-d₆) δ=10.37 (s, 1H), 8.45 (s, 1H), 8.02 (d, J=9.0 Hz, 1H), 7.42 (dd, J=9.0, 3.0 Hz, 1H), 7.28 (d, J=3.0 Hz, 1H), 7.26 (s, 1H), 6.51 (s, 1H), 5.41 (s, 2H), 5.23 (s, 2H), 1.86 (m, 2H), 0.87 (t, J=7.2 Hz, 3H). ¹³C-NMR (150 MHz, DMSO-d₆) δ=173.06, 157.42, 157.11, 150.64, 149.86, 146.38, 143.67, 131.10, 130.39, 130.17, 129.84, 123.57, 118.59, 109.30, 96.51, 72.90, 65.69, 50.64, 30.71, 8.20. HRMS calculated for C₂₀H₁₆N₂O₅, 364.1059; found, 364.1075.

11-hydroxycamptothecin: ¹H-NMR (600 MHz, DMSO-d₆) δ=10.44 (s, 1H), 8.54 (s, 1H), 7.97 (d, J=7.8 Hz, 1H), 7.38 (d, J=3.0 Hz, 1H), 7.30 (s), 7.27 (dd, J=8.4, 2.4 Hz, 1H), 6.50 (s, 1H), 5.42 (s, 2H), 5.22 (s, 2H), 1.86 (m, 2H), 0.88 (t, J=7.2 Hz, 3H). ¹³C-NMR (150 MHz, DMSO-d₆) δ=172.99, 159.79, 157.35, 152.81, 150.46, 146.35, 139.99, 138.61, 131.73, 130.21, 129.86, 121.05, 119.10, 110.33, 96.89, 72.87, 65.73, 50.59, 30.74, 8.24. HRMS calculated for C₂₀H₁₆N₂O₅, 364.1059; found, 364.1070.

10,11-dihydroxycamptothecin: ¹H-NMR (600 MHz, DMSO-d₆) δ=10.35 (s, 1H), 10.15 (s, 1H), 8.34 (s, 1H), 7.37 (s, 1H), 7.28 (s, 1H), 7.26 (s, 1H), 6.46 (s, 1H), 5.40 (s, 2H), 5.18 (s, 2H), 1.88 (m, 2H), 0.88 (m, 3H).

7-ethyl-10-hydroxycamptothecin: ¹H-NMR (600 MHz, DMSO-d₆) δ=10.30 (s, 1H), 8.02 (d, J=9.0 Hz, 1H), 7.40 (m, 2H), 7.24 (s, 1H), 6.49 (s, 1H), 5.41 (d, J=2.4 Hz, 2H), 5.26 (s, 2H), 3.07 (m, 2H), 1.85 (m, 2H), 1.29 (t, J=7.8 Hz, 3H), 0.88 (t, J=7.2 Hz, 3H). ¹³C-NMR (150 MHz, DMSO-d₆) δ=172.56, 156.85, 156.72, 150.06, 148.84, 146.43, 142.73, 131.55, 128.18, 128.00, 122.37, 117.99, 104.76, 95.78, 72.40, 69.77, 65.24, 49.45, 30.21, 22.29, 13.36, 7.76. HRMS calculated for C₂₂H₂₀N₂O₅, 392.1372; found, 392.1370.

7-ethyl-11-hydroxycamptothecin: ¹H-NMR (600 MHz, DMSO-d₆) δ=10.39 (s, 1H), 8.14 (d, J=9.0 Hz, 1H), 7.38 (d, J=2.4 Hz, 1H), 7.29 (dd, J=9.0, 2.4 Hz, 1H), 7.21 (s, 1H), 6.52 (s, 1H), 5.43 (s, 2H), 5.27 (s, 2H), 3.24 (m, 2H), 1.88 (m, 2H), 1.30 (t, J=7.8 Hz, 3H), 0.88 (t, J=7.2 Hz, 3H). ¹³C-NMR (150 MHz, DMSO-d₆) δ =172.55, 156.83, 156.16, 150.64, 149.98, 146.37, 145.36, 129.04, 128.10, 125.28, 120.86, 120.16, 110.68, 96.35, 72.39, 69.77, 65.26, 49.31, 30.27, 22.22, 14.03, 7.75. HRMS calculated for C₂₂H₂₀N₂O₅, 392.1372; found, 392.1383.

Topotecan-11: ¹H-NMR (600 MHz, DMSO-d₆) δ=8.65 (s, 1H), 8.12 (d, J=9.0 Hz, 1H), 7.64 (d, J=9.0 Hz, 1H), 7.48 (s, 1H), 5.44 (s, 2H), 5.27 (s, 2H), 4.59 (s, 2H), 2.85 (s, 6H), 1.89 (m, 2H), 0.89 (t, J=7.2 Hz, 3H). ¹³C-NMR (150 MHz, DMSO-d₆) δ=172.42, 159.39, 156.87, 152.33, 150.12, 148.51, 145.70, 132.30, 131.41, 127.40, 122.52, 120.03, 119.03, 109.46, 97.29, 80.32, 72.60, 65.46, 63.02, 61.09, 50.21, 30.77, 8.02. HRMS calculated for C₂₃H₂₃N₃O₅, 421.1638; found, 421.1643.

Irinotecan-11: ¹H-NMR (600 MHz, DMSO-d₆) δ=8.31 (d, J=9.6 Hz, 1H), 7.88 (d, J=2.4 Hz, 1H), 7.56 (dd, J=9.0, 2.4 Hz, 1H), 7.32 (s, 1H), 6.52 (s, 1H), 5.44 (s, 2H), 5.34 (s, 2H), 3.24 (m, 3H), 1.86 (m, 2H), 1.32 (t, J=7.8 Hz, 3H), 0.88 (t, J=7.2 Hz, 3H), 1.23-4.08 (19H). ¹³C-NMR (150 MHz, DMSO-d₆) δ=172.53, 156.77, 152.67, 152.41, 149.95, 145.94, 145.60, 127.85, 125.16, 124.29, 123.45, 120.29, 119.10, 108.08, 96.76, 72.41, 65.29, 62.21, 61.75, 61.56, 52.31, 49.55, 49.43, 45.75, 43.38, 42.85, 30.29, 26.90, 25.29, 22.35, 20.75, 14.04, 7.79. HRMS calculated for C₃₃H₃₈N₄O₆, 586.2791; found, 586.2814.

9-bromo-10-hydroxycamptothecin: ¹H-NMR (600 MHz, DMSO-d₆) δ=11.18 (s, 1H), 8.74 (s, 1H), 8.08 (d, J=9.0 Hz, 1H), 7.63 (d, J=9.0 Hz, 1H), 7.29 (s, 1H), 5.42 (s, 2H), 5.30 (s, 2H), 1.86 (m, 2H), 0.88 (m, 3H). ¹³C-NMR (150 MHz, DMSO-d₆) δ=172.69, 157.06, 154.00, 150.26, 150.16, 145.52, 143.91, 131.62, 130.25, 128.99, 128.74, 122.47, 118.80, 103.95, 96.62, 75.26, 65.40, 50.71, 30.45, 7.90. HRMS calculated for C₂₀H15BrN₂O₅, 442.0164; found, 442.0159.

12-bromo-11-hydroxycamptothecin: ¹H-NMR (600 MHz, DMSO-d₆) δ=11.05 (s, 1H), 8.62 (s, 1H), 8.00 (d, J=9.0 Hz, 1H), 7.46 (d, J=9.0 Hz, 1H), 7.36 (s, 1H), 5.44 (s, 2H), 5.27 (s, 2H), 4.73 (s, 1H), 1.87 (m, 2H), 0.89 (m, 3H). ¹³C-NMR (150 MHz, DMSO-d₆) δ=172.70, 157.04, 156.79, 153.11, 150.30, 146.91, 142.07, 132.23, 128.80, 128.53, 127.81, 123.81, 119.16, 106.28, 96.99, 72.70. 65.50, 50.30, 30.52, 8.06. HRMS calculated for C₂₀H15BrN₂O₅, 442.0164; found, 442.0159.

All citations are hereby incorporated by reference.

The present invention has been described with regard to one or more embodiments. However, it will be apparent to persons skilled in the art that a number of variations and modifications can be made without departing from the scope of the invention as defined in the claims.

1. A cytochrome P450 monooxygenase capable of oxidizing a monoterpenoid indole alkaloid (MIA) substrate, wherein the MIA substrate comprises a quinoline moiety or an indole moiety.

2.-4. (canceled)

5. The cytochrome P450 monooxygenase of claim 1 wherein the camptothecin hydroxylase is CPT 9-hydroxylase (CPT9H), CPT 10-hydroxylase (CPT10H) or CPT 11-hydroxylase (CPT11H).

6. The cytochrome P450 monooxygenase of claim 5 wherein the camptothecin hydroxylase is derived from Camptotheca acuminata, Ophiorrhiza pumila or Nothapodytes nimmoniana or wherein the camptothecin hydroxylase is derived from an orthologue or homolog of the camptothecin hydroxylase from Camptotheca acuminata, Ophiorrhiza pumila or Nothapodytes nimmoniana.

7. The cytochrome P450 monooxygenase of claim 1, wherein the cytochrome P450 monooxygenase comprising a sequence with 80-100% identity to SEQ ID NO: 3, 4, 8, 9, 10, 14, 15, 16, 18, 20, 22, 24, 26, 28 or 30, or an active fragment or variant thereof.

8. A nucleic acid comprising a nucleotide sequence encoding the cytochrome P450 monooxygenase of claim 1.

9. A transgenic host or host cell comprising the cytochrome P450 monooxygenase of claim 1.

10. (canceled)

11. A method of producing a hydroxylated monoterpenoid indole alkaloid (MIA), wherein the MIA comprises a quinoline moiety or an indole moiety, the method comprising:
(a) providing a first cytochrome P450 monooxygenase, wherein the first cytochrome P450 monooxygenase comprises the cytochrome P450 monooxygenase of any one of claims 1-7;
(b) contacting a monoterpenoid indole alkaloid (MIA) substrate with the first cytochrome P450 monooxygenase under conditions suitable for oxidation or hydroxylation of the MIA substrate to produce a hydroxylated MIA.

12. (canceled)

13. The method of claim 11, wherein the MIA substrate is camptothecine, 7-ethylcamptothecin, 9-amino-camptothecin, 10-hydroxycamptothecin, 9-nitro-camptothecin, evodiamine or ellipticine.

14. The method of claim 11 wherein the method further comprises contacting the hydoxylated MIA with a second cytochrome P450 monooxygenase, wherein the second cytochrome P450 monooxygenase comprises the cytochrome P450 monooxygenase of claim 1, under conditions suitable for oxidation or hydroxylation of the hydroxylated MIA to produce a dihydroxylated MIA.

15. (canceled)

16. A method of producing a hydroxylated monoterpenoid indole alkaloid (MIA), the method comprising:
(a) providing the transgenic host or host cell of claim 9;
(b) incubating the host or host cell under condition suitable for the expression of the cytochrome P450 monooxygenases;
(c) contacting the cytochrome P450 monooxygenases with a MIA substrate under conditions suitable for oxidation or hydroxylation of the MIA substrate to produce a hydroxylated MIA.

17. The method of claim 16, wherein the contacting in step (c) comprises an in vitro contact or the contacting in step (c) comprises an in vivo contact within the host or host cell.

18. The method of claim 11, further comprising the step of recovering the hydroxylated MIA.

19.-20. (canceled)

21. The method of claim 11, wherein the hydroxylated MIA is a 9-hydroxycamptothecinoid, a 10-hydroxycamptothecinoid, a 11-hydroxycamptothecinoid, 10,11-dihydroxycamptothecinoid, a 7-ethyl-10-hydroxycamptothecinoid, a 9-amino-hydroxycamptothecinoid, a 9-nitro-hydroxycamptothecinoid or a combination thereof.

22. The method of claim 11, wherein the hydroxylated MIA is further processed into a MIA derivative.

23. The method of claim 22 wherein the MIA derivative is a camptothecin analogue selected from: 9-[(dimethylamino)methyl]-10-hydroxycamptothecin (topotecan); 12-[(dimethyl amino)methyl]-11-hydroxycamptothecin (topotecan-11), 7-ethyl-10-[4-(1-piperidino)-1-piperidino]carbonyloxycamptothecin (irinotecan); 7-ethyl-11-[4-(1-piperidino)-1-piperidino]carbonyloxycamptothecin (irinotecan-11); 7-ethyl-10-hydroxycamptothecin; 7-ethyl-11-hydroxycamptothecin; 9-bromo-10-hydroxycamptothecin; 12-bromo-10-hydroxycamptothecin; 9-amino-10-hydroxycamptothecin or 9-amino-11-hydroxycamptothecin.

24. A monoterpenoid indole alkaloid (MIA) derivative produced by the method of claim 22, wherein the MIA derivative is 12-[(dimethylamino)methyl]-11-hydroxycamptothecin (topotecan-11), 7-ethyl-11-[4-(1-piperidino)-1-piperidino]carbonyloxycamptothecin (irinotecan-11), 10,11-dihydroxycamptothecin, 12-bromo-11-hydroxycamptothecin, 10-hydroxy-11-methoxycamptothecin or 11-hydroxy-10-methoxycamptothecin.

25. A camptothecin derivative having the chemical structure of Formula I:

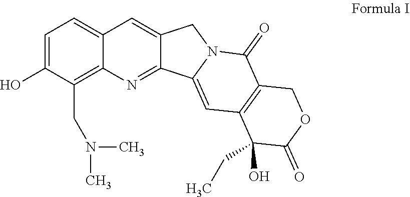 [SMILES: CC[C@@]1(O)C(=O)OCc2c1cc1n(c2=O)Cc2cc3ccc(O)c(CN(C)C)c3nc2-1]

26.-27. (canceled)

28. A camptothecin derivative having the chemical structure of Formula IV:

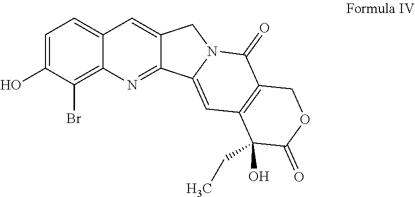 [SMILES: CC[C@@]1(O)C(=O)OCc2c1cc1n(c2=O)Cc2cc3ccc(O)c(Br)c3nc2-1]

29.-31. (canceled)

32. A pharmaceutical composition comprising an effective amount of the MIA derivative of claim 24.

33. A method of treating cancer in a subject, comprising administering to the subject a therapeutically effective amount of the pharmaceutical composition of claim 32.


SEQUENCE LISTING (38 sequences)

SEQ ID NO: 1 - DNA, 1539 bp, Camptotheca acuminata
atggagaacttgtactactgccttgctctcctactatcaattcttttcatattcagacatttcttccgtcatagttcaaagttaccaccaagtccgtttgcccttcctatcatcggccatctccatctcatcaggaattctttgcaccaagtactagagtgcttggcatctcaatatggtccaattttatttctcaaatttggcacccgctctattcttgttgtgtcttctccatccgctgttgaggaatgcctcattaagaatgatattatatttgcaaaccgtcctcggagcatgattttagatctctctagttttaattatagtatattttcatgggctccatatggtcattactggcgaagcctccgccgccttgctgttgttgaactcttcacatcgcgcagccttcagacgtcttccaacatccgtaaagaggaaattcataaccttctctgtcacctcttcaaattctcaaaaagtggaactcaaaaactccagttgaaatattggttctctctattgacattcaatattataacaaggctggtagctgggaagcggtgtgttagagatgcagttgcaggcacggattcgggtaaacaaattcttgaagacctcgaggggaagttcacttcaaaaatgccatttaatatgtgtgatttctttccaattttgaggtggtttggttacaaagggttggagaaaattctgattacgttgcacaaggagagagatgaattcatgcaaggtttgatagatgaggttagacgaaagagaacatgttctgccaatatcaatagtgtaacaaacagagcaaagacaacattgattgaagctctcttgtccctccaagaatcagaacctgacttcttttctgatactatcatcaaaagtatcatttcagacatgttttttgcagggccagaaacatcaacaatcactttagaatgggcaatgtcacttcttctaaatcatccagaggtattgggaaagttgagagcagagattgatgatcatgttggacatggacgccttctagatgactcggatcttgggaagcttccctatctccgttgcatcatcaatgagaccctcagattatatcctccaacaccacttctattaccacactgttcatctgaagattgcattgtggggggatatgaaataccacaaggtacaatcctgtgggtgaatgcttgggccatgcatagagatcccaagttgtgggaggagccaaccaagttcaagcctgagagatttgaaggcatggaagggagagaaaggtataaatttatgccatttggaattgggagaagagcttgtccaggtgctagtatggccatccggacagtttcattggcattgggtgcacttattcaatgttttgaatgggaaaacgttgggccggataaaagggagatgagccagggtcgacttactttgcccaaggccgagtctttggaggctgtgtctattccacgccccagtgcagtgaaagtcctctcccagcttgaaggcacttgtttccgttag

SEQ ID NO: 2 - DNA, 1539 bp, Camptotheca acuminata
atggagaacttgtactactgccttgctctcctactatcaattcttttcatattcagacatttcttccatcatagttcaaagttaccaccaagtccatttgcctttcctatcatcggccatctccatctcatcaggaattctttccaccaagtactagagtgcttggcatctcaatatggtccaattttattcctcaaatttggcatccgctctattcttgttgtgtcatcaccatccgttgttgaggaatgttttattaagaatgatattatatttgcaaaccgtcctcggaatatgctttcagatatctctagttataattatagtacgatcgtaggggctccatatggtcattactggcggagcctccgccgccttgctagtgttgaattcttctcattgaatagcctccagaagtcttctaacatccgtgaagaggaaattcataaccttctctatcacctcttcaaattctcaaaaagtggaactcaaaaagtccagttgaaatattggttctctctattgacattcaatataataacgaggctggtagctgggaagcggtgtgttagagatgcggttgcaggcatggatttggggaaacaaattcttgaagaactcaaggggaagttcgtttcgatcatgccattgaatatgtgtgatttctttccaattttgaggtggtttggttacaaagggctggagaaaaatctgattacgttgcacaaggagagagatgaattcttgcaggacttgataaatgaggttagacgaaagagaacatgttctgccaatatcaatattgtaacaaacaaagcaaagacaacattgattggaactctcttgtccttccaagaatcagaacctgacttcttttctgatactatcatcaaaagtatcatttcagacatgttttttgcaggatcagaaacatcagcaatcactctagaatgggcaatgtcacttcttctaaatcatccagaggtattgggaaagttgagagcagagattgatgatcatgttggacatggacgccttctagatgactcggatcttgtgaagcttccctatcttcgttgcatcatcaatgaaaccctcagattatatcctccaacaccacttctattacctcactgttcatctgtagattgcactgtggggggatatgaaataccacaaggtacaatcctgtgggtgaatgcttgggccatgcatagagatcccaagttatgggaggagccaaccaagttcaagcctgagagatttgaaggcatggaagggagagaaaggtacaaatttattccatttggaattgggagaagagcttgtccaggtgctagtatgggcatccggacagtttcattggctttgggcgcacttattcagtgttttcaatgggaaaacgttgggcaggataaaagggagatgagtccggttcgacttacgttgcccaaggccgagtctttggaggctatgtgtattccacgccccagtgcaatgaaagtcctctcccagcttgaagacacttgtttcagttag

SEQ ID NO: 3 - protein, 511 aa, Camptotheca acuminata
MENLYYCLALLLSILFIFRHFFRHSSKLPPSPFALPIIGHLHLIRNSLHQVLECLASQYGPILFLKFGTRSILVVSSPSAVEECLIKNDIIFANRPRSMILDLSSFNYSIFSWAPYGHYWRSLRRLAVVELFTSRSLQTSSNIRKEEIHNLLCHLFKFSKSGTQKLQLKYWFSLLTFNIITRLVAGKRCVRDAVAGTDSGKQILEDLEGKFTSKMPFNMCDFFPILRWFGYKGLEKILITLHKERDEFMQGLIDEVRRKRTCSANINSVTNRAKTTLIEALLSLQESEPDFFSDTIIKSIISDMFFAGPETSTITLEWAMSLLLNHPEVLGKLRAEIDDHVGHGRLLDDSDLGKLPYLRCIINETLRLYPPTPLLLPHCSSEDCIVGGYEIPQGTILWVNAWAMHRDPKLWEEPTKFKPERFEGMEGRERYKFMPFGIGRRACPGASMAIRTVSLALGALIQCFEWENVGPDKREMSQGRLTLPKAESLEAVSIPRPSAVKVLSQLEGTCF

SEQ ID NO: 4 - protein, 512 aa, Camptotheca acuminata
MENLYYCLALLLSILFIFRHFFHHSSKLPPSPFAFPIIGHLHLIRNSFHQVLECLASQYGPILFLKFGIRSILVVSSPSVVEECFIKNDIIFANRPRNMLSDISSYNYSTIVGAPYGHYWRSLRRLASVEFFSLNSLQKSSNIREEEIHNLLYHLFKFSKSGTQKVQLKYWFSLLTFNIITRLVAGKRCVRDAVAGMDLGKQILEELKGKFVSIMPLNMCDFFPILRWFGYKGLEKNLITLHKERDEFLQDLINEVRRKRTCSANINIVTNKAKTTLIGTLLSFQESEPDFFSDTIIKSIISDMFFAGSETSAITLEWAMSLLLNHPEVLGKLRAEIDDHVGHGRLLDDSDLVKLPYLRCIINETLRLYPPTPLLLPHCSSVDCTVGGYEIPQGTILWVNAWAMHRDPKLWEEPTKFKPERFEGMEGRERYKFIPFGIGRRACPGASMGIRTVSLALGALIQCFQWENVGQDKREMSPVRLTLPKAESLEAMCIPRPSAMKVLSQLEDTCFS

SEQ ID NO: 5 - DNA, 1557 bp, Ophiorrhiza pumila
atggagaatctctactattacttagtgtcaatcttcttgtgtggtgttttcctgattctatccaaacaattgttgttcaacaagaacaagaagttacctcctagtcctcgtgttcttccaataattggccatctccatctcatcaagaacgaattctatgaagattttacttcattatcatctacatacggtccagttttctttctccgatttggctgccggtcctatgttgttgtgtcttctccatctgctgttggagagtgtttcacaaagaatgatattatacttgcaaaccgtcctaagaccatggctggggacaggttgacctataactatggatcatttggagtggctccttatggggatatatggagggttcttcgtcgcctcactgttgttgaatctttatctttcaacagcctccaaaagtcctcaaatatcagggaagaagaaattcagatgattgttcgttcactctatcgagtctcaaagaatggaagccaacgagttgatttgaactattggatttcagtttttacactcaatgttattatgaggatggttactggaagatgctcaattagagaggaagatgctggagacgagttggggaagcaaatagttaaagaattcaaagacaactttgctacagccctttcaatgagcttgtgcgacttcttcccgatattaaggtggtttggttacaaagggctggaaaagagaatgatcattttgcacaagaagagagatgcattccttcagggtttagtagatgaacttcgatcaaataaatctaatttttctccttccggcactggaatgaacgaagagaagaagaaggcattaattcaatccctcctttctcatcaggaactagaacctgattttctcaaagatgactctataaagagtattgcattgtccatctttctagcaggaagagaaacgtcatccatgaccattgaatgggctatgtcactcttactgaatcaccaggaagcaatgcagaagttaaggactgaaatcgacaacaacgtaggacacaaaagattgttggatgaatcggatattccaaagcttccttatctgcgttgtgtagtggatgagacgatgagactgtatcctgcagcaccactgcttcttcctcattatgcgtctgaaaattgtagagtttgtgactatgacattccaaaaggtacgactgttttaactaatgcttgggccatacacagggatccaaaactctgggatatgcctgaaaagttcatgccagagagatttgaggctaaacaaatagggggaaaagaagagttcaatttcaagtttctaccatttgggatagggaggagagcatgtcccggagccaatttggccattcggaacgtttctttggcattgggtgcattgttacagtgcttttattgggaaaaagttggagagaaggaaggcgatatggacagtaagaacgatgatagagtcactttgcagaaggccaaacccttggaggccatttgttttccacgccaagaatcaatccaacttctctcgcaactctga

SEQ ID NO: 6 - DNA, 1443 bp, Ophiorrhiza pumila
atgtcaacccgagttctttgggataagattcctatcagactaagagttttaatcctactgcaactctaccagacttcatcagttttctttctccgatttggctgccggtcctatgttgttgtgtcttctccatctgctgttggagagtgtttcacaaagaatgatattatacttgcaaaccgtcctaagaccatggctggggacaggttgacctataactatggatcatttggagtggctccgtatggggatatatggagggttcttcgtcgcctcactgttgttgaatctttatctttcaacagcctccaaaagtcctctaatatcagggaagaagaaattcagatgattgttcgttcactctatcgagtctcaaagaatggaagccaacgagttgatttgaactattggatttcagtttttacactcaatgttattatgaggatggttactggaagatgctcaattagagaggaagatgctggagacgagttggggaagcaaatagttaaagaattcaaagacaactttgctacagccctttcaatgagcttgtgcgacttcttcccgatattaaggtggtttggttacaaagggctggaaaagagaatgatcattttgcacaagaagagagatgcattccttcagggtttagtagatgaacttcgatcaaataaatctaatttttctccttccggcactggaatgaacgaagagaagaagaaggcattaattcaatccctcctttctcatcaggaactagaacctgattttctcaaagatgactctataaagagtattgcattgtccatctttctagcaggaagagaaacgtcatccatgaccattgaatgggctatgtcactcttactgaatcaccaggaagcaatgcagaagttaaggactgaaatcgacaacaacgtaggacacaaaagattgttggatgaatcggatattccaaagcttccttatctgcgttgtgtagtggatgagacgatgagactgtatcctgcagcaccactgcttcttcctcattatgcgtctgaaaattgtagagtttgtgactatgacattccaaaaggtacgactgttttaactaatgcttgggccatacacagggatccaaaactctgggatatgcctgaaaagttcatgccagagagatttgaggctaaacaaatagggggaaaagaagagttcaatttcaagtttctaccatttgggatagggaggagagcatgtcccggagccaatttggccattcggaacgtttctttggcattgggtgcattgttacagtgcttttattgggaaaaagttggagagaaggaaggcgatatggacagtaagaacgatgatagagtcactttgcagaaggccaaacccttggaggccatttgttttccacgccaagaatcaatccaacttctctcgcaactctga

SEQ ID NO: 7 - DNA, 3042 bp, Ophiorrhiza pumila
atggagaatctctactactacttagtgtcaatcttcttgtgtggttttttcctgatcctatccaaacaattgtttttcaacaagaacaagaagttacctcctagtcctcgtgctcttccaataattggccatctccatctcatcaagaacgaactctatgaagattttacttcattatcatctacatacggtccagttttctttctccgatttggctgccggtcctatgttgttgtgtcttctccatctgctgttgaagagtgtttcacaaagaatgatattatacttgcaaaccgtgataagaccatggctggggacaggttgacctataactatggaacatttgcaatggctccttatggggatatatggagggttcttcgtcgcctcactgttgttgaatctttatctttcaacagactccaaaagtcctcaaatatcagggaagaagaaattcagatgattgttcgttcactctttcgagtctcaaagaatggaagccaacgagttgatttgaactattggatttcagtttttacactcaatgttattatgaggatggttactggaagatgctcaattagagaggaagatgctggagacgagttggggaagcaaatagttaaagaattcaaagacaactttgctacaggcctttcaatgaacttgtgcgacttcttcccgatattaaggtggtttggttacaaagggctggaaaagagaatgatcattttgcacaagaagagagatgcattccttcagggtttagtagatgaacttcgatcaaataaatctaatttttctccttccggcactggaatgaacgaagagaagaagaaggcattaattgaatccctcctttctcatcaggaactagaacctgattttctcaaagatgactctataaagagtattgcattgtccatctttatagcaggaagagaaacatcatccatgaccattgaatgggctatgtcactcttactgaatcacccggaagcaatgcacaagttaaggactgaaatcgacaacaacgtaggacacaaaagattgttggatgaatcggatattccaaagcttccttatctgcgttgtgtcgtggatgagacattgagactgtatcctccagcaccactgcttctacctcattatgcatctgaaaattgtagagtttgggactatgacattccaaaaggtacgactgttttagctaatgcttgggccatacacagggatccaaaactctgggatatgcctgaaaagttcatgccagagagatttgaggctaaacaattaggggaaaaagaagagttcaatttcaagtttctaccatttgggatagggaggagagcatgtcccggagccaatttgggcattcggaacgtttctttggcattgggtgcattgttacagtgcttttattgggaaaaagttggagagaaggaaggcgatatggacatgatagtggatagagccatagagttctattttgccatggagaatctctactactacttagtctcaatctttttgtgttgctttttcgtgatcctattcctatccaaacaattgctgttcaacaagaacaagaagttgccacccagtcctcctgctcttccaataattggccatctccatctcatcaagaacgaactctatcgagatttaacttcattatcatctacatacggtccagttttctttctcaaatttggttgccggtcctatgttgttgtgtcttctccatctgccgttgaagagtgcttcacaaagaatgataatatacttgcaaaccgtcctaacaccatggcttcggacatttttacctataactactcaacaattggatcggctccttatgggaatttatggagggttcttcgtcgcctcactgttgctgaatctttatcatccaacagccttcagaagtcctcaaatatcagggaagaagaaattcagatgattgttcgttcactctttcgaatctcaaagaatggaagccaacgagttgatttgaactactggatttcagtttttacactcaatattattacgaggatgattactggaagatgctcaattagagaggaggatgccggagatgagttggggaagcaaatagctaaagaattcaaagataggtttgcttcaggcactgcaatgaacttgtgcgacttctttccgatattaaggtggtttggttacaaagggttggaaaagaaaatgatcagtttgtacaagaagagagatgcattccttcagggtttagtagataaatttcgatcagataaatctaacaacgaagagaagaagaagaccataattgaatctctcctctctcatcaggaagaactagaactaaagcctgattttctctcagatggcttaataaagagtactgcgctgtccatctttatagcaggaagagaaacatcatccctgaccattgaatgggctatgtcactcttactgaaacacccgaaagcaatgcacaagttaaggactgaaatcgacaacaatgtaggacacaaaagattgttggatgaatcggatattccaaagcttccttatctgcgttgtgtcgtggatgagacattgagactgtatcctccagcaccactgcttctacctcattatgcatctgaaaattgtagagtttgggactatgacattccaaaaggtacgactgttttagctaacgcttgggccatacacagggatccaaaactctgggatatgcctgaaaagttcatgccagagagatttgaggctaaacaattaggggaaaaagaagagttcaatttcaagtttctaccatttgggatagggaggagagcatgtcccggagccaatttgggcattcggaacgtttctttggcattgggtgcattgttacagtgcttttattgggacaaagttggagaaaaggaaggtgatatggacactaacaacgacgataaactcactttgcataaggccaaaccctgcgaggccatgtgttttccacgccaagaatcaatccaacttctctcgcaactctga

SEQ ID NO: 8 - protein, 518 aa, Ophiorrhiza pumila
MENLYYYLVSIFLCGVFLILSKQLLFNKNKKLPPSPRVLPIIGHLHLIKNEFYEDFTSLSSTYGPVFFLRFGCRSYVVVSSPSAVGECFTKNDIILANRPKTMAGDRLTYNYGSFGVAPYGDIWRVLRRLTVVESLSFNSLQKSSNIREEEIQMIVRSLYRVSKNGSQRVDLNYWISVFTLNVIMRMVTGRCSIREEDAGDELGKQIVKEFKDNFATALSMSLCDFFPILRWFGYKGLEKRMIILHKKRDAFLQGLVDELRSNKSNFSPSGTGMNEEKKKALIQSLLSHQELEPDFLKDDSIKSIALSIFLAGRETSSMTIEWAMSLLLNHQEAMQKLRTEIDNNVGHKRLLDESDIPKLPYLRCVVDETMRLYPAAPLLLPHYASENCRVCDYDIPKGTTVLTNAWAIHRDPKLWDMPEKFMPERFEAKQIGGKEEFNFKFLPFGIGRRACPGANLAIRNVSLALGALLQCFYWEKVGEKEGDMDSKNDDRVTLQKAKPLEAICFPRQESIQLLSQL

SEQ ID NO: 9 - protein, 480 aa, Ophiorrhiza pumila
MSTRVLWDKIPIRLRVLILLQLYQTSSVFFLRFGCRSYVVVSSPSAVGECFTKNDIILANRPKTMAGDRLTYNYGSFGVAPYGDIWRVLRRLTVVESLSFNSLQKSSNIREEEIQMIVRSLYRVSKNGSQRVDLNYWISVFTLNVIMRMVTGRCSIREEDAGDELGKQIVKEFKDNFATALSMSLCDFFPILRWFGYKGLEKRMIILHKKRDAFLQGLVDELRSNKSNFSPSGTGMNEEKKKALIQSLLSHQELEPDFLKDDSIKSIALSIFLAGRETSSMTIEWAMSLLLNHQEAMQKLRTEIDNNVGHKRLLDESDIPKLPYLRCVVDETMRLYPAAPLLLPHYASENCRVCDYDIPKGTTVLTNAWAIHRDPKLWDMPEKFMPERFEAKQIGGKEEFNFKFLPFGIGRRACPGANLAIRNVSLALGALLQCFYWEKVGEKEGDMDSKNDDRVTLQKAKPLEAICFPRQESIQLLSQL

SEQ ID NO: 10 - protein, 515 aa, Ophiorrhiza pumila
MENLYYYLVSIFLCCFFVILFLSKQLLFNKNKKLPPSPPALPIIGHLHLIKNELYRDLTSLSSTYGPVFFLKFGCRSYVVVSSPSAVEECFTKNDNILANRPNTMASDIFTYNYSTIGSAPYGNLWRVLRRLTVAESLSSNSLQKSSNIREEEIQMIVRSLFRISKNGSQRVDLNYWISVFTLNIITRMITGRCSIREEDAGDELGKQIAKEFKDRFASGTAMNLCDFFPILRWFGYKGLEKKMISLYKKRDAFLQGLVDKFRSDKSNNEEKKKTIIESLLSHQEELELKPDFLSDGLIKSTALSIFIAGRETSSLTIEWAMSLLLKHPKAMHKLRTEIDNNVGHKRLLDESDIPKLPYLRCVVDETLRLYPPAPLLLPHYASENCRVWDYDIPKGTTVLANAWAIHRDPKLWDMPEKFMPERFEAKQLGEKEEFNFKFLPFGIGRRACPGANLGIRNVSLALGALLQCFYWDKVGEKEGDMDTNNDDKLTLHKAKPCEAMCFPRQESIQLLSQL

SEQ ID NO: 11 - DNA, 3042 bp, Ophiorrhiza pumila; identical to SEQ ID NO: 7

SEQ ID NO: 12 - DNA, 1443 bp, Ophiorrhiza pumila; identical to SEQ ID NO: 6

SEQ ID NO: 13 - DNA, 1557 bp, Ophiorrhiza pumila; identical to SEQ ID NO: 5

SEQ ID NO: 14 - protein, 515 aa, Ophiorrhiza pumila; identical to SEQ ID NO: 10

SEQ ID NO: 15 - protein, 480 aa, Ophiorrhiza pumila; identical to SEQ ID NO: 9

SEQ ID NO: 16 - protein, 518 aa, Ophiorrhiza pumila; identical to SEQ ID NO: 8

SEQ ID NO: 17 - DNA, 1536 bp, Nothapodytes nimmoniana
atggagatgctttacttctaccttatttttctggtctcagttctcctgatattcaaacacatcttccattttaacaaaagtaaattaccaccaagtcctccatatattccgataattggccacctctacctcataaagggtagtatccaccaagcacttcagtctctgtcatcaaaatatggtccaattctattcctccggctcggcgtccggtccatgttggttgtctcttctccctctgccgtggaagaatgcttcaccaagaacgacatcatatttgcaaaccggccccgaaccttggccggcgacctgttgacttacaactacagagctttcgtgtggactccgtacggacatatttggcggagcctccgccgtctctcggtggttgaactcttctcttcaaccagcgtccacaggtcttcagcagttcgtgaagatgaaatccgaaccctcgttcgacatctctataaagtatcaaagagtgggaatccaaaggtggaattcaagtactggttctcaatttgtttgttcaataccataacgaggattgtcgccgggagacaggttgtaccggaggaagacgcaggcggggaggccgggcggcgaattatggcagaccttagagagagattctttacgaacgtcggaatgaatatgtgcgatttccttccaattctgaggtggtttggttacaaagggctggaaaaaaaattgatggtagcgttcaaaaggagggacgagttcttgcagggcctactagatgagtttcgattaaagaaaatgaattcctcatctcagaaacatgtgaaagatggaaaagagaaaggtccgttgatagaaactctgttgtcccttcgtgaatcagagcctgagttctacaccgttgatgtcatcaaaagtttaatgctggtaatgtttgtggctggaacagagacaactgcaactactgtagagtgggcaatgtcacttcttctaacacaccctgaaacacttgacaagctaagaacagagattgacaacaatgtcagggaagaacgactactaaccgacatggatctttctaaacttccttatctccgttgtgttatcaacgaagccctcagattgtaccccccagtgccacttctattaccacatttctcatctaaagattgtacaattggagggcatgtgatacccgaaggtacaatcctagttgttaattcttgggcattgcaaagggatcccaacgtttgggaggagccacacaagttcaagccagagagatttgagatggaggaggaaaaagaagggtttggttataaattcgttccgtttggggtagggaggagggcatgccctggagtcaatatgggcatgagggcagctttgttggcacttggtacactgattcaatgttttgagtgggaaaaggttggccaatttgagatggaaatgaggtacaataatggagtaactttgcagaaggctaaaccctttgaagctaattgcaaaccaagacaaaattttgttcaactccttggtcagctttga

SEQ ID NO: 18 - protein, 511 aa, Nothapodytes nimmoniana
MEMLYFYLIFLVSVLLIFKHIFHFNKSKLPPSPPYIPIIGHLYLIKGSIHQALQSLSSKYGPILFLRLGVRSMLVVSSPSAVEECFTKNDIIFANRPRTLAGDLLTYNYRAFVWTPYGHIWRSLRRLSVVELFSSTSVHRSSAVREDEIRTLVRHLYKVSKSGNPKVEFKYWFSICLFNTITRIVAGRQVVPEEDAGGEAGRRIMADLRERFFTNVGMNMCDFLPILRWFGYKGLEKKLMVAFKRRDEFLQGLLDEFRLKKMNSSSQKHVKDGKEKGPLIETLLSLRESEPEFYTVDVIKSLMLVMFVAGTETTATTVEWAMSLLLTHPETLDKLRTEIDNNVREERLLTDMDLSKLPYLRCVINEALRLYPPVPLLLPHFSSKDCTIGGHVIPEGTILVVNSWALQRDPNVWEEPHKFKPERFEMEEEKEGFGYKFVPFGVGRRACPGVNMGMRAALLALGTLIQCFEWEKVGQFEMEMRYNNGVTLQKAKPFEANCKPRQNFVQLLGQL

SEQ ID NO: 19 - DNA, 1536 bp, Nothapodytes nimmoniana; identical to SEQ ID NO: 17

SEQ ID NO: 20 - protein, 511 aa, Nothapodytes nimmoniana; identical to SEQ ID NO: 18

SEQ ID NO: 21 - DNA, 1545 bp, Camptotheca acuminata
atggagaacatatactactaccttgctctcctcttgtctgttctcttcatgttcaaacatttcttccatcacaatcggaagttaccaccaagtccgcttgcgcttccaattattggccacctccaccttatcaagaagttgctacaccagtcactagagtgtctttcatcccgatatggtccaattttatttctccaatttggctcccgttccgttgttgctttatcttctccatctgccgttgaagaatgcttcaccaaaaatgacataatatttgcaaaccggcctcgaacaatggctggggatcatttcacttacaattatactgcctttgtatgggctccatatggtcatctctggcggagtctccgccgtctgactgtcattgagctcttctcttcaaacagccttcagaagtcttcttttgttcgtgagggggaaattggtaatcttctatgtcacctgttcaaattctcaaacaatggaactcaaaaagtcgagttgaagtattggttctctcttttggcatttaatatcatgatgaagatgattgctggaaagcgatgtgttagagatgaggttgcaggcatggaggcagggaagcaaattcttgaagatctcaggggaaagttcgtttcaaccacaccattgaatatttgtgatttctttccaattttgaggtggcttggctacaaagggctgaagaagagtatgataaggttgcacaagaagagagatgaattcttgcagggtttgatagatgagtttcgaattaaaagcagttcttctgccaataccaatgctataatgcacagggtacaaaaggtaacattgattgagaaactcttgtctctgcaagaagcagaacctgacttctattcggatgacgttatcaaaagtatcatattggtaacttttgtggcaggtaccgaaacatcagcagtcactatagaatgggcaatgtcacttcttctaaataatccacaggcattggtgaaggtgaaagcagagatttccagtcatgtcggatttgagcgcttgctaaatgactctgatcttcccaagctacattatctccgttgtgtcatcaatgagacgctcagattatatcctccggtgccactcctgttaccacactactcatcgaaagattgcactttaggggggtatgaaattccacaaggtacaattctaactgtgaatgcttgggcaatgcatagggatcccaaggtgtgggaagatcccaccaagttcaaccctgagagatttgaagttgttcaaggggaaagagaagggttcaaatttattccatttggagtggggaggagagcttgtccaggtgcagctatggccttgcggacagtttcattagctttgggtgcactgattcaatgttttgaatgggaaaaggttggacaggagaatatggagacgagtcagggaggactgactttgcccaaggctgggtgtttggaggctgtgtgcattccacgccaagattcgattaaactgctatcccaacttgaaagccattgttctgattaa

SEQ ID NO: 22 - protein, 514 aa, Camptotheca acuminata
MENIYYYLALLLSVLFMFKHFFHHNRKLPPSPLALPIIGHLHLIKKLLHQSLECLSSRYGPILFLQFGSRSVVALSSPSAVEECFTKNDIIFANRPRTMAGDHFTYNYTAFVWAPYGHLWRSLRRLTVIELFSSNSLQKSSFVREGEIGNLLCHLFKFSNNGTQKVELKYWFSLLAFNIMMKMIAGKRCVRDEVAGMEAGKQILEDLRGKFVSTTPLNICDFFPILRWLGYKGLKKSMIRLHKKRDEFLQGLIDEFRIKSSSSANTNAIMHRVQKVTLIEKLLSLQEAEPDFYSDDVIKSIILVTFVAGTETSAVTIEWAMSLLLNNPQALVKVKAEISSHVGFERLLNDSDLPKLHYLRCVINETLRLYPPVPLLLPHYSSKDCTLGGYEIPQGTILTVNAWAMHRDPKVWEDPTKFNPERFEVVQGEREGFKFIPFGVGRRACPGAAMALRTVSLALGALIQCFEWEKVGQENMETSQGGLTLPKAGCLEAVCIPRQDSIKLLSQLESHCSD

SEQ ID NO: 23 - DNA, 1476 bp, Camptotheca acuminata
atggatatggatatggatacggggctcgtcttctgcataatagttatcatcttctccatccttttcatttccaaatttctattgccacaaaggaaaaactttccaccgagtccactcgctcttcccatactcggccatctccacctcctcaagaatccggtgcacagggcgctccagtctctgtccaatcagcacggcccaatcctactgctgcggttcggatcccgccctgtccttgtcgtctcgtctccgtcggccgcccaacaatgcttcaccgctgaaaacgacgttatcttcgcaaaccgacccaacaccctcggcggcaaacacttcggctacaactacactactcttgggtggtccccctacggcgaccggtggcgcgatctccgccgtatcaccaccatccaaatcttctcctccaagagtctccaggattctgccacggtccgaagagaggaggtccggtttatcacccgccagctgtttctgggatccgaaggatcgacccagaaggtgaacgtgcaatatctggccttccagctgaccttcaacttgacgatgaagatggtcgctggaaaaaggtgttcaagggcgaaggagatattcgctccgatgatgcggatgaataaattagatttcttaccctttttgaagtggtttggtctcaaaggatcggagaatgggttggtgaagttacagaaggcaagagatgcattcttgcagggcttgatcgatgagtatcgaccggagagggaagtggacaagaagaagacgatgatcgagactttgttgtcttttcaagaagaagaccctgaatttttcacggaaaatacagtcaagggcatcatggtgctactatttacagcgggaacagatactgtagctcgcacaatggaatgggcaatgtcacttctcctgaatcacccagaagtactgcaaaaggccagaagcgaaatagacaatcatgtaaagccacatcgtctgctagaggactctgatctttccaaactaccttatctacgttgcatcatcaacgaaactcttcgattatttcctgttgcaccacttctcgtacctcatttttcatcagaagactgcttagtagagagattccatgttccacgaggaacaattttgttggtcaatgcttgggccattcatagggatcccagtgtctgggaagagcccaccaagtttaagccagagaggtttgaaggaattgaaggggaacgagaagggttcaagttcataccatttggggtggggaggaggggatgtcctggtgctggcttggctctgcgtttgcttgggttggccttggggacattgattcagtgctttgagtgggaaagggttgggtctgaattggtggacttgaccgagggcagtgggataactttgctaaaggttaagccattagaggccatgtatagacctcgccggtccatgaccgctctcctttctcaactttga

SEQ ID NO: 24 - protein, 491 aa, Camptotheca acuminata
MDMDMDTGLVFCIIVIIFSILFISKFLLPQRKNFPPSPLALPILGHLHLLKNPVHRALQSLSNQHGPILLLRFGSRPVLVVSSPSAAQQCFTAENDVIFANRPNTLGGKHFGYNYTTLGWSPYGDRWRDLRRITTIQIFSSKSLQDSATVRREEVRFITRQLFLGSEGSTQKVNVQYLAFQLTFNLTMKMVAGKRCSRAKEIFAPMMRMNKLDFLPFLKWFGLKGSENGLVKLQKARDAFLQGLIDEYRPEREVDKKKTMIETLLSFQEEDPEFFTENTVKGIMVLLFTAGTDTVARTMEWAMSLLLNHPEVLQKARSEIDNHVKPHRLLEDSDLSKLPYLRCIINETLRLFPVAPLLVPHFSSEDCLVERFHVPRGTILLVNAWAIHRDPSVWEEPTKFKPERFEGIEGEREGFKFIPFGVGRRGCPGAGLALRLLGLALGTLIQCFEWERVGSELVDLTEGSGITLLKVKPLEAMYRPRRSMTALLSQL

SEQ ID NO: 25 - DNA, 1179 bp, Camptotheca acuminata
atggtcattactggcgaagcctccgccgccttgctattgttgaactcttcacatcgaacagccttcagaagtcttccaacatccgtaaagaggaaattcataaccttctctgtcacctcttcaaattctcaaaaagtggagttgaaatattggttttttcgattgacattcaatattataacaaggctggtagctgggaaacaatgtgttagagatgcacttgcaggcacagatttggggaaacaaattcttgaagacctcgaggggaagttcggttcaaaaatgccattgaatatgtgtgatttctttccaattttgaggtggtttggttacaaagggctggagaaaagtctgacagtgtggcacaaggagagagatgaatttatgcaaggtttgatagatgaggttagacgaaagagaacctgttctgccaatatcaataatataacaaacagagcaaagacaacattgattgaagctctcttgtccctccaagaatcacaacctgacttcttttctgatactatcatcaaaagtatcatttcagacatgttttttgcagggccagaaacatcagcaatcactctagaatgggcaatgtcacttcttctaaatcatccagaggtactgcgaaagttaagagcagggattgatgatcatgttggacatggacgccttctagatgactcggatcttgtgaagcttccctatctccgttgcatcatcaatgagaccctcagattatatcctccaacaccacttctattaccacactgttcatctgaggattgcactgtggggggatatgaaataccacaaggtacaatcctgtgggtgaatgcttgggccatgcatagagatcccaagttatgggaggagccaaccaagttcaagcctgagagatttgaaggcatggaagggagagaaaggaacaaatttattccatttggaattgggagaagagcttgtccaggtgctagtatgggcatccggacagtttcattggctttgggtgcacttattcagtgttttgaatgggaaaacgttgggcagaagaaaatggagatgagccaaggtcgacttactttgcccaaggccgagtctttggaggctacgtgtattccacgccctagtgcaatgaaagtcctctcccagcttgaagacacttgtttcagttag

SEQ ID NO: 26 - protein, 392 aa, Camptotheca acuminata
MVITGEASAALLLLNSSHRTAFRSLPTSVKRKFITFSVTSSNSQKVELKYWFFRLTFNIITRLVAGKQCVRDALAGTDLGKQILEDLEGKFGSKMPLNMCDFFPILRWFGYKGLEKSLTVWHKERDEFMQGLIDEVRRKRTCSANINNITNRAKTTLIEALLSLQESQPDFFSDTIIKSIISDMFFAGPETSAITLEWAMSLLLNHPEVLRKLRAGIDDHVGHGRLLDDSDLVKLPYLRCIINETLRLYPPTPLLLPHCSSEDCTVGGYEIPQGTILWVNAWAMHRDPKLWEEPTKFKPERFEGMEGRERNKFIPFGIGRRACPGASMGIRTVSLALGALIQCFEWENVGQKKMEMSQGRLTLPKAESLEATCIPRPSAMKVLSQLEDTCFS

SEQ ID NO: 27 - DNA, 1527 bp, Camptotheca acuminata
atggacacactgtatacatctcttgcattaatattagccacatatttcttcatcaaacacttcgtcattcgcaagatccaaaacaaaccaccgagtccattcccatcgctgcccattctcggccacctccacctcttgaagaagcccctccaccgaaccttggcccatatatcggcccgttacggcagtattattctcctccatttcggatcacgtccagtcgtcgtagtctcatctccctcagcagcggaggaatgcctcaccaagaacgacatcatcttcgccaatcgccctcgcctcctcgccggaaaataccttggctacaaccatacctccctcgcgtgggccccctatagcgaccactggcggaacctccgccggatcgcgtcgctcgaaattctgtcatcccataggctgcagatgttatccggcatacgctccgacgaggtgcgttcggtggttcgtagactttcccgggcttccgcagatgatcgggtggacatgaaaaaggtattcttcgagctgatgcttaacgtgatgatgagaatgattgctggaaagaggtattacggcgagaacgtggcggaggtagagcaggggacgcggtttcgcgagatcgtggtggagacattcctgctttctggagccacaaacatgggggactttttgcccattttgaattgggtgggagtgacgggatcggagaagcggttgatggcgttgcagaagaagagagatgcgtttatgcaggaattgatagaagagcatagaagaggaatggggatcgataatggcgattcagatgagcagggagagaaaaagaagacgatgattgcagttttgttatccctgcaagaaacggaacctgattattacaaggatgaaattatcagaggcatcacgctggttctgttagctgcaggaactgatacttcagctgggaccatggagtgggcactttcacttttgttgaacaatccagaagttctaaaaaaggcacagattgaaattgataataaggttggacaaaaccgtttggtcaatgaatcagacatagctgacctcccttatctccgctgcatcctcaacgagacctttcggatgttcccggtaggcccattattattacctcatgaatcatcagaagattgcacggtcggaggtttccacatcccacgtggcactatgctaatgattaatttgtgggccatacaaaatgaccccaagatttgggaggacccaagaaagttcaagccagaacggtttgaaggactggaaggggtaagagatggtttcaaattgatgccttttgggtcaggcaggagagggtgtcctggggagggtctggccatgcgaatgcttggctttacattagggtcattgattcagtgctttgattgggaaagggttggcaaggacttggtggacttgactgaagggcctgggctcaccatgcccaaggctcaacccttggtggctaagtgccggccacgtgcaacaatgttgaaccttctgtctcaaatttga

SEQ ID NO: 28 - protein, 508 aa, Camptotheca acuminata
MDTLYTSLALILATYFFIKHFVIRKIQNKPPSPFPSLPILGHLHLLKKPLHRTLAHISARYGSIILLHFGSRPVVVVSSPSAAEECLTKNDIIFANRPRLLAGKYLGYNHTSLAWAPYSDHWRNLRRIASLEILSSHRLQMLSGIRSDEVRSVVRRLSRASADDRVDMKKVFFELMLNVMMRMIAGKRYYGENVAEVEQGTRFREIVVETFLLSGATNMGDFLPILNWVGVTGSEKRLMALQKKRDAFMQELIEEHRRGMGIDNGDSDEQGEKKKTMIAVLLSLQETEPDYYKDEIIRGITLVLLAAGTDTSAGTMEWALSLLLNNPEVLKKAQIEIDNKVGQNRLVNESDIADLPYLRCILNETFRMFPVGPLLLPHESSEDCTVGGFHIPRGTMLMINLWAIQNDPKIWEDPRKFKPERFEGLEGVRDGFKLMPFGSGRRGCPGEGLAMRMLGFTLGSLIQCFDWERVGKDLVDLTEGPGLTMPKAQPLVAKCRPRATMLNLLSQI

SEQ ID NO: 29 - DNA, 1542 bp, Camptotheca acuminata
atggagaagttgtactactgccttgctcttctactatcagttcttctcatattcaaacatttcttccatcatagaacaaagttaccaccaagtccatttgctcttcctatcatcggccatctccatctcatcaggaattctttccatcaaatactagagtgcttggcatcacaatatggtccaattttattcctcaaagttggaatccgctctattcttgttgtgtcgtctccatccgttgttgaggaatgttttactaagaatgatattatatttgcaaaccgtcctcggaatatgctttcagatatctctagttataattatagtacgatcgcatgggctccatatggtcattactggcggagcctccgccgccttactgttgttgaattcttctcattgaatagcctccagaagtcttctaacatccgtgaagaggaaattcataaccttctctctcacctcttcaaattctcaaaaagtggaactcaaaaagtccagttgaaatattggttctctctattgactttcaatataataacgaggctggtagctgggaagcggtgtgttagagatgcggttgcaggcaaggatttggggaaacaaattcttgaagagctcaaggggaagttcgtttcgaacatgccattgaatatgtgtgatttctttccaattttgaggtggtttggttacaaagggctggagaaaagtctgattatgttgctgcagaaggagagagatgaattcttgcagggtttgatagatgaggttagacgaaagagaacctgttctgccaatatcaatattgtaacaaacagagcaaagacaacattgattgaaactctcttgtccctccaagaatcagaacctgacttcttttctgatactgtcatcaaaagtatcatttcagtcatgttttttgcagggccggaaacatcagcaattactctggaatgggcaatatcgcttcttctaaataatccagaggtactggggaagttaagagcagagattgatgatcatgttggacatggacgccttctagatgactcggatcttgtgaagcttccctatctccgttgcatcatcaatgagaccctcagattatatcctccggcaccacttctattaccacgttgttcatcagaagattgcactgttgggggatatgaaataccacaaggtacaattctgttggtgaatgcttgggccatgcatagagatcccaagttgtgggaggagccaaccaagttcaagcctgagagatttgaaggcatggaagggagagaagggtacaaatttattccatttggagttgggagaagagcttgtccaggtgctagaatgggcatctggacagtttcactggctttgggtgctcttgctcagtgttttgaatgggaaaaggttgtggaggataaaatggagatgagccagggtcgactaactatgtccaaggccgagtctttggaggctctgtgtattccacgccacagtgcaatgacactcctctcccagcttgaagacacttcctttatttag

SEQ ID NO: 30 - protein, 513 aa, Camptotheca acuminata
MEKLYYCLALLLSVLLIFKHFFHHRTKLPPSPFALPIIGHLHLIRNSFHQILECLASQYGPILFLKVGIRSILVVSSPSVVEECFTKNDIIFANRPRNMLSDISSYNYSTIAWAPYGHYWRSLRRLTVVEFFSLNSLQKSSNIREEEIHNLLSHLFKFSKSGTQKVQLKYWFSLLTFNIITRLVAGKRCVRDAVAGKDLGKQILEELKGKFVSNMPLNMCDFFPILRWFGYKGLEKSLIMLLQKERDEFLQGLIDEVRRKRTCSANINIVTNRAKTTLIETLLSLQESEPDFFSDTVIKSIISVMFFAGPETSAITLEWAISLLLNNPEVLGKLRAEIDDHVGHGRLLDDSDLVKLPYLRCIINETLRLYPPAPLLLPRCSSEDCTVGGYEIPQGTILLVNAWAMHRDPKLWEEPTKFKPERFEGMEGREGYKFIPFGVGRRACPGARMGIWTVSLALGALAQCFEWEKVVEDKMEMSQGRLTMSKAESLEALCIPRHSAMTLLSQLEDTSFI

SEQ ID NO: 31 - DNA, 42 bp, Artificial Sequence
  Forward Primer sequence of pESC-Leu2d-32245
cactaaagggcggccaacaaaatggagaagttgtactactgc

SEQ ID NO: 32 - DNA, 44 bp, Artificial Sequence
  Reverse primer sequence pESC-Leu2d-32245
cactaaagggcggccaacaaaatggagaagttgtactactgcct

SEQ ID NO: 33 - DNA, 44 bp, Artificial Sequence
  Forward primer sequence pESC-Leu2d-32236
cactaaagggcggccaacaaaatggagaacttgtactactgcct

SEQ ID NO: 34 - DNA, 34 bp, Artificial Sequence
  Reverse primer sequence pESC-Leu2d-32236
atccatcgatactagacggaaacaagtgccttca

SEQ ID NO: 35 - DNA, 42 bp, Artificial Sequence; identical to SEQ ID NO: 31
  Forward primer sequence pESC-Leu2d-32245

SEQ ID NO: 36 - DNA, 40 bp, Artificial Sequence
  Reverse primer sequence pESC-Leu2d-32245
atccatcgatactagaataaaggaagtgtcttcaagctgg

SEQ ID NO: 37 - DNA, 44 bp, Artificial Sequence; identical to SEQ ID NO: 33
  Forward primer sequence pESC-Leu2d-32229

SEQ ID NO: 38 - DNA, 39 bp, Artificial Sequence
  Reverse primer sequence pESC-Leu2d-32229
atccatcgatactagactgaaacaagtgtcttcaagctg